(19) United States
(12) Patent Application Publication
(10) Pub. No.: US 2022/0047637 A1
(43) Pub. Date: Feb. 17, 2022

(54) SYSTEMS AND METHODS FOR THE TREATMENT OF HEMOGLOBINOPATHIES

(71) Applicant: EDITAS MEDICINE, INC., Cambridge, MA (US)

(72) Inventors: Edouard AUPEPIN DE LAMOTHE-DREUZY, Boston, MA (US); KaiHsin CHANG, Medfield, MA (US); Jack HEATH, Winchester, MA (US)

(73) Assignee: EDITAS MEDICINE, INC., Cambridge, MA (US)

(21) Appl. No.: 17/298,514

(22) PCT Filed: Nov. 27, 2019
(86) PCT No.: PCT/US19/63766

§ 371 Date: May 28, 2021

Related U.S. Application Data
(60) Provisional application No. 62/773,089, filed on Nov. 29, 2018.

(51) Int. Cl. A61K 35/28; C12N 15/11; C12N 9/22

(52) U.S. Cl. CPC A61K 35/28; C12N 15/111; C12N 9/22; C12N 2310/20; C12N 2310/346; C12N 2320/31; C12N 2310/315

(57) ABSTRACT

Genome editing systems, guide RNAs, and CRISPR-mediated methods are provided for altering portions of the HBG1 and HBG2 loci, portions of the erythroid specific enhancer of the BCL11A gene, or a combination thereof, in cells and increasing expression of fetal hemoglobin.

### CROSS-REFERENCE TO RELATED APPLICATIONS

The present application claims the benefit of U.S. Provisional Application No. 62/773,089, filed Nov. 29, 2018, the contents of which are hereby incorporated by reference in their entirety.

### SEQUENCE LISTING

This application contains a Sequence Listing, which was submitted in ASCII format via EFS-Web, and is hereby incorporated by reference in its entirety. The ASCII copy, created on Nov. 27, 2019, is named SequenceListing.txt and is 356 KB in size.

### FIELD

This disclosure relates to genome editing systems and methods for altering a target nucleic acid sequence, or modulating expression of a target nucleic acid sequence, and applications thereof in connection with the alteration of genes encoding hemoglobin subunits and/or treatment of hemoglobinopathies.

### BACKGROUND

Hemoglobin (Hb) carries oxygen in erythrocytes or red blood cells (RBCs) from the lungs to tissues. During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. The average adult makes less than 1% HbF out of total hemoglobin (Thein 2009). The α-hemoglobin gene is located on chromosome 16, while the 0-hemoglobin gene (HBB), A gamma (Aγ)-globin chain (HBGJ, also known as gamma globin A), and G gamma (Gγ)-globin chain (HBG2, also known as gamma globin G) are located on chromosome 11 within the globin gene cluster (also referred to as the globin locus).

Mutations in HBB can cause hemoglobin disorders (i.e., hemoglobinopathies) including sickle cell disease (SCD) and beta-thalassemia (β-Thal). Approximately 93,000 people in the United States are diagnosed with a hemoglobinopathy. Worldwide, 300,000 children are born with hemoglobinopathies every year (Angastiniotis 1998). Because these conditions are associated with HBB mutations, their symptoms typically do not manifest until after globin switching from HbF to HbA.

SCD is the most common inherited hematologic disease in the United States, affecting approximately 80,000 people (Brousseau 2010). SCD is most common in people of African ancestry, for whom the prevalence of SCD is 1 in 500. In Africa, the prevalence of SCD is 15 million (Aliyu 2008). SCD is also more common in people of Indian, Saudi Arabian and Mediterranean descent. In those of Hispanic-American descent, the prevalence of sickle cell disease is 1 in 1,000 (Lewis 2014).

SCD is caused by a single homozygous mutation in the HBB gene, c.17A>T (HbS mutation). The sickle mutation is a point mutation (GAG>GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less elastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood, the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, Hepatitis B, and Hepatitis C. Subjects may also suffer from acute chest crises and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-Thal is estimated to affect approximately 1 in 100,000 people worldwide. Its prevalence is higher in certain populations, including those of European descent, where its prevalence is approximately 1 in 10,000. β-Thal major, the more severe form of the disease, is life-threatening unless treated with lifelong blood transfusions and chelation therapy. In the United States, there are approximately 3,000 subjects with β-Thal major. β-Thal intermedia does not require blood transfusions, but it may cause growth delay and significant systemic abnormalities, and it frequently requires lifelong chelation therapy. Although HbA makes up the majority of hemoglobin in adult RBCs, approximately 3% of adult hemoglobin is in the form of HbA2, an HbA variant in which the two γ-globin chains are replaced with two delta (Δ)-globin chains. δ-Thal is associated with mutations in the A hemoglobin gene (HBD) that cause a loss of HBD expression. Co-inheritance of the HBD mutation can mask a diagnosis of β-Thal (i.e., β/δ-Thal) by decreasing the level of HbA2 to the normal range (Bouva 2006). β/δ-Thal is usually caused by deletion of the HBB and HBD sequences in both alleles. In homozygous (δo/δo βo/βo) patients, HBG is expressed, leading to production of HbF alone.

Like SCD, β-Thal is caused by mutations in the HBB gene. The most common HBB mutations leading to β-Thal are: c. −136C>G, c.92+1G>A, c.92+6T>C, c.93-21G>A, c.118C>T, c.316-106C>G, c.25 26delAA, c.27 28insG, c.92+5G>C, c.118C>T, c.135delC, c.315+1G>A, c. −78A>G, c.52A>T, c.59A>G, c.92+5G>C, c.124 127delTTCT, c.316-197C>T, c. −78A>G, c.52A>T, c.124 127delTTCT, c.316-197C>T, c. −138C>T, c. −79A>G, c.92+5G>C, c.75T>A, c.316-2A>G, and c.316-2A>C. These and other mutations associated with β-Thal cause mutated or absent β-globin chains, which causes a disruption of the normal Hb α-hemoglobin to β-hemoglobin ratio. Excess α-globin chains precipitate in erythroid precursors in the bone marrow.

In β-Thal major, both alleles of HBB contain nonsense, frameshift, or splicing mutations that leads to complete absence of β-globin production (denoted β⁰/β⁰). β-Thal major results in severe reduction in β-globin chains, leading to significant precipitation of α-globin chains in RBCs and more severe anemia.

β-Thal intermedia results from mutations in the 5′ or 3′ untranslated region of HBB, mutations in the promoter region or polyadenylation signal of HBB, or splicing mutations within the HBB gene. Patient genotypes are denoted βo/β+ or β+/β+. represents absent expression of a β-globin chain; β+ represents a dysfunctional but present β-globin chain. Phenotypic expression varies among patients. Since there is some production of β-globin, β-Thal intermedia results in less precipitation of α-globin chains in the erythroid precursors and less severe anemia than β-Thal major. However, there are more significant consequences of erythroid lineage expansion secondary to chronic anemia.

Subjects with β-Thal major present between the ages of 6 months and 2 years, and suffer from failure to thrive, fevers, hepatosplenomegaly, and diarrhea. Adequate treatment includes regular transfusions. Therapy for β-Thal major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-Thal intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-Thal intermedia includes splenectomy, folic acid supplementation, hydroxyurea therapy, and radiotherapy for extramedullary masses. Chelation therapy is used in subjects who develop iron overload.

Life expectancy is often diminished in β-Thal patients. Subjects with β-Thal major who do not receive transfusion therapy generally die in their second or third decade. Subjects with β-Thal major who receive regular transfusions and adequate chelation therapy can live into their fifth decade and beyond. Cardiac failure secondary to iron toxicity is the leading cause of death in β-Thal major subjects due to iron toxicity.

A variety of new treatments are currently in development for SCD and β-Thal. Delivery of an anti-sickling HBB gene via gene therapy is currently being investigated in clinical trials. However, the long-term efficacy and safety of this approach is unknown. Transplantation with hematopoietic stem cells (HSCs) from an HLA-matched allogeneic stem cell donor has been demonstrated to cure SCD and β-Thal, but this procedure involves risks including those associated with ablation therapy, which is required to prepare the subject for transplant, increases risk of life-threatening opportunistic infections, and risk of graft vs. host disease after transplantation. In addition, matched allogeneic donors often cannot be identified. Thus, there is a need for improved methods of managing these and other hemoglobinopathies.

### SUMMARY

Provided herein are genome editing systems, guide RNAs, and CRISPR-mediated methods for altering one or more γ-globin genes (e.g., HBG1, HBG2, or HBG1 and HBG2), the erythroid specific enhancer of the BCL11A gene (BCL11Ae), or a combination thereof, and increasing expression of fetal hemoglobin (HbF). In certain embodiments, genome editing systems, guide RNAs, and CRISPR-mediated methods may alter a 13 nucleotide (nt) target region that is 5′ of the transcription site of the HBG1, HBG2, or HBG1 and HBG2 gene (“13 nt target region”). In certain embodiments, one or more gRNAs comprising a targeting domain set forth in SEQ ID NOs:251-901 or 940-942 may be used to introduce alterations in the 13 nt target region. In certain embodiments, one or more gRNAs comprising a sequence set forth in SEQ ID NOs:970-979 may be used to introduce alterations in the 13 nt target region. In certain embodiments, genome editing systems, guide RNAs, and CRISPR-mediated methods may alter a GATA1 binding motif in BCL11Ae that is in the +58 DNase I hypersensitive site (DHS) region of intron 2 of the BCL11A gene (“GATA1 binding motif in BCL11Ae”). In certain embodiments, one or more gRNAs comprising a targeting domain set forth in SEQ ID NOs:952-955 may be used to introduce alterations in the GATA1 binding motif in BCL11Ae. In certain embodiments, one or more gRNAs may be used to introduce alterations in the GATA1 binding motif in BCL11Ae and one or more gRNAs may be used to introduce alterations in the 13 nt target region of HBG1 and/or HBG2.

Provided herein in certain embodiments are gRNAs for use in CRISPR/Cas-mediated methods of increasing expression (i.e., transcriptional activity) of one or more γ-globin genes (e.g., HBG1, HBG2, or HBG1 and HBG2). In certain embodiments, the gRNAs may be generated via in vitro transcription or chemical synthesis. In certain embodiments, the gRNA may comprise one or more modifications. In certain embodiments, the one or more modifications may be selected from the group consisting of phosphorothioate and 2′-O-methyl groups. In certain embodiments, the one or more modifications may be at or near the 5′ end, 3′ end, or both of the gRNA. In certain embodiments, the gRNA may comprise a truncated form of a full-length gRNA (e.g., SEQ ID NO:970). In certain embodiments, the gRNA may comprise, consist essentially of, or consist of a sequence selected from the group consisting of SEQ ID NOs: 339 and 970-979. In certain embodiments, the gRNA may differ by no more than 3 nucleotides from a sequence selected from the group consisting of SEQ ID NOs: 339 and 970-979.

Provided herein in certain embodiments are the use of optional genome editing system components such as oligonucleotide donor templates. In certain embodiments, donor templates for use in targeting the 13 nt target region may include, without limitation, donor templates encoding alterations (e.g., deletions) of HBG1 c. −114 to −102, HBG1 c. −225 to −222, and/or HBG2 c. −114 to −102. In certain embodiments, 5′ and 3′ homology arms and exemplary full-length donor templates encoding deletions such as c. −114 to −102 are also presented below (e.g., SEQ ID NOS: 904-909, 990-995). In certain embodiments, the template nucleic acid may be a single stranded oligodeoxynucleotide (ssODN). In certain embodiments, the template nucleic acid may be a positive strand or a negative strand. In certain embodiments, the ssODN may comprise a 5′ homology arm, a replacement sequence, and a 3′ homology arm. In certain embodiments, the 5′ homology arm may be about 25 to about 200 nucleotides or more in length, e.g., at least about 25, 50, 75, 100, 125, 150, 175, or 200 nucleotides in length; the replacement sequence may comprise 0 nucleotides in length; and the 3′ homology arm may be about 25 to about 200 nucleotides or more in length, e.g., at least about 25, 50, 75, 100, 125, 150, 175, or 200 nucleotides in length. In certain embodiments, the 5′ homology arm may comprise about 50 to 100 bp, e.g., 55 to 95, 60 to 90, 70 to 90, or 80 to 90 bp, homology 5′ to the 13 nt target region and the 3′ homology arm may comprise about 50 to 100 bp, e.g., 55 to 95, 60 to 90, 70 to 90, or 80 to 90 bp, homology 3′ to the 13 nt target region. In certain embodiments, the 13 nt target region may be HBG1 c. −114 to −102 (e.g., nucleotides 2824-2836 of SEQ ID NO:902 (HBG1)), HBG2 c. −114 to −102 (e.g., nucleotides 2748-2760 of SEQ ID NO:903 (HBG2)), or a combination thereof. In certain embodiments, the ssODN may comprise one or more phosphorothioates.

Also provided herein are methods of treating hemoglobinopathy in a subject in need thereof. In certain embodiments, the methods may comprise: a) isolating a population of CD34+ or hematopoietic stem cells from the subject; b) modifying the population of isolated cells ex vivo by delivering a first RNP complex comprising a first guide RNA (gRNA) and a first RNA-guided nuclease to the population of isolated cells, thereby to affect an alteration in a promoter of an HBG gene in one or more cells in the population; and c) administering the modified population of cells to the subject, thereby to treat the hemoglobinopathy in the subject. In certain embodiments, the hemoglobinopathy may be severe sickle cell disease (SCD) or thalassemia. In certain embodiments, the thalassemia may be selected from the group consisting of β-thalassemia, δ-thalassemia, and β/δ-thalassemia. In certain embodiments, the RNA-guided nuclease may be an S. pyogenes Cas9. In certain embodiments, the RNA-guided nuclease may be a nickase, and optionally lacks RuvC activity. In certain embodiments, the first gRNA may comprise any of the targeting domain sequences described herein. For example, the first gRNA may comprise a first targeting domain complimentary to a sequence within positions c. −1,114 to −114 of a human HBG1 or HBG2 gene. In certain embodiments, the first gRNA may comprise a first targeting domain complimentary to a sequence within positions c. −214 to −114 of a human HBG1 or HBG2 gene. In certain embodiments, the first gRNA may comprise a first targeting domain complimentary to a sequence within positions c. −102 to −52 of a human HBG1 or HBG2 gene. In certain embodiments, the first gRNA may comprise a first targeting domain comprising a targeting domain listed in Table 7. In certain embodiments, the first gRNA may comprise a first targeting domain that differs by no more than 3 nucleotides from a targeting domain listed in Table 7. In certain embodiments, the methods may further comprise delivering a second RNP complex comprising a second gRNA and a second RNA-guided nuclease to the population of isolated cells. In certain embodiments, the second gRNA may comprise any of the targeting domain sequences described herein. In certain embodiments, the first and second guide RNAs may comprise first and second targeting domains complimentary to first and second sequences on opposite sides of positions of a 13 nt target region of a human HBG1 or HBG2 gene, wherein one or both of the first and second sequences optionally overlaps the 13 nt target region of the human HBG1 or HBG2 gene.

Also provided herein are ex vivo methods of increasing the level of fetal hemoglobin (HbF) in a human cell by genome editing using a first RNP complex comprising a first gRNA and a first RNA-guided nuclease to affect an alteration in a promoter of an HBG gene, thereby to increase expression of HbF. In certain embodiments, the first gRNA may comprise any of the targeting domain sequences described herein. In certain embodiments, the first gRNA may comprise a first targeting domain comprising a targeting domain listed in Table 7. In certain embodiments, the first gRNA may comprise a first targeting domain that differs by no more than 3 nucleotides from a targeting domain listed in Table 7. In certain embodiments, the methods further comprise using a second RNP complex comprising a second gRNA and a second RNA-guided nuclease to affect an alteration in a promoter of an HBG gene, thereby to increase expression of HbF. In certain embodiments, the second gRNA may comprise any of the targeting domain sequences described herein. In certain embodiments, the first and second guide RNAs may comprise first and second targeting domains complimentary to first and second sequences on opposite sides of positions of a 13 nt target region of a human HBG1 or HBG2 gene, wherein one or both of the first and second sequences optionally overlaps the 13 nt target region of the human HBG1 or HBG2 gene.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1 depicts, in schematic form, HBG1 and HBG2 gene(s) in the context of the β-globin gene cluster on human chromosome 11. FIG. 1. Each gene in the β-globin gene cluster is transcriptionally regulated by a proximal promoter. While not wishing to be bound by any particular theory, it is generally thought that A_{γ} and/or G_{γ} expression is activated by engagement between the proximal promoter with the distal strong erythroid-specific enhancer, the locus control region (LCR). Long-range transactivation by the LCR is thought to be mediated by alteration of chromatin configuration/confirmation. The LCR is marked by 4 erythroid specific Dnase I hypersensitive sites (HS1-4) and 2 distal enhancer elements (5′ HS and 3′ HS1). β-like gene globin gene expression is regulated in a developmental stage-specific manner, and expression of globin genes changes coincide with changes in the main site of blood production.

FIGS. 2A-2B depict HBG1 and HBG2 genes, coding sequences (CDS) and small deletions and point mutations in and upstream of the HBG1 and HBG2 proximal promoters that have been identified in patients and associated with elevation of fetal hemoglobin (HbF). Core elements within the proximal promoters (CAAT box, 13 nt sequence) that have been deleted in some patients with hereditary persistence of fetal hemoglobin (HPFH). The ‘target sequence’ region of each locus, which has been screened for gRNA binding target sites, is also identified.

FIGS. 3A-C show data from gRNA screening for incorporation of the 13 nt deletion in human K562 erythroleukemia cells. FIG. 3A Gene editing as determined by T7E1 endonuclease assay analysis (referred to interchangeably as a “T7E1 analysis”) of HBG1 and HBG2 locus-specific PCR products amplified from genomic DNA extracted from K562 cells after electroporation with DNA encoding S. pyogenes-specific gRNAs and plasmid DNA encoding S. pyogenes Cas9. FIG. 3B Gene editing as determined by DNA sequence analysis of PCR products amplified from the HBG1 locus in genomic DNA extracted from K562 cells after electroporation with DNA encoding the indicated gRNA and Cas9 plasmid. FIG. 3C Gene editing as determined by DNA sequence analysis of PCR products amplified from the HBG2 locus in genomic DNA extracted from K562 cells after electroporation with DNA encoding the indicated gRNA and Cas9 plasmid. For FIG. 3B-C, the types of editing events (insertions, deletions) and subtypes of deletions (13 nt target partially [12 nt HPFH] or fully [13-26 nt HPFH] deleted, other sequences deleted [other deletions]) are indicated by the differently shaded/patterned bars.

FIG. 4 depicts results of gene editing in human cord blood (CB) and human adult CD34⁺ cells after electroporation with RNPs complexed to in vitro transcribed S. pyogenes gRNAs that target a specific 13 nt sequence for deletion (HBG sgRNAs Sp35 and Sp37). FIG. 4A depicts the percentage of indels detected by T7E1 analysis of HBG1 and HBG2 specific PCR products amplified from gDNA extracted from CB CD34⁺ cells treated with the indicated RNPs or donor matched untreated control cells (n=3 CB CD34⁺ cells, 3 separate experiments). Data shown represent the mean and error bars correspond to standard deviation across the 3 separate donors/experiments. FIG. 4B depicts the percentage of indels detected by T7E1 analysis of HBG2 specific PCR product amplified from gDNA extracted from CB CD34⁺ cells or adult CD34⁺ cells treated with the indicated RNPs or donor matched untreated control cells (n=3 CB CD34⁺ cells, n=3 mobilized peripheral blood (mPB) CD34⁺ cells, 3 separate experiments). Data shown represent the mean and error bars correspond to standard deviation across the 3 separate donors/experiments. FIG. 4C (Top panel) depicts indels as detected by T7E1 analysis of HBG2 PCR products amplified from gDNA extracted from human CB CD34⁺ cells electroporated with HBG Sp35 RNP or HBG Sp37 RNP+/−ssODN (unmodified or with PhTx modified 5′ and 3′ ends). The lower left panel shows the level of gene editing as determined by Sanger DNA sequence analysis of gDNA from cells edited with HBG Sp37 RNP and ssODN. The lower right panel shows the specific types of deletions detected within total deletions.

FIG. 5 depicts gene editing of HBG in adult human mobilized peripheral blood (mPB) CD34⁺ cells and induction of fetal hemoglobin in erythroid progeny of RNP treated cells after electroporation of mPB CD34⁺ cells with HBG Sp37 RNP+/−ssODN encoding the 13 nt deletion. FIG. 5A depicts the percentage of indels detected by T7E1 analysis of HBG2 PCR product amplified from gDNA extracted from mPB CD34+ cells treated with the RNP or donor matched untreated control cells. FIG. 5B depicts the fold change in HBG mRNA expression in day 7 erythroblasts that were differentiated from RNP treated and untreated donor matched control mPB CD34⁺ cells. mRNA levels are normalized to GAPDH and calibrated to the levels detected in untreated controls on the corresponding days of differentiation.

FIG. 6 depicts the ex vivo differentiation potential of RNP treated and untreated mPB CD34⁺ cells from the same donor. FIG. 6A shows hematopoietic myeloid/erythroid colony forming cell (CFC) potential, where the number and subtype of colonies are indicated (GEMM: granulocyte-erythroid-monocyte-macrophage colony, E: erythroid colony, GM: granulocyte-macrophage colony, M: macrophage colony, G: granulocyte colony). FIG. 6B depicts the percentage of Glycophorin A expressed over the time course of erythroid differentiation as determined by flow cytometry analysis at the indicated time points and for the indicated samples.

FIG. 7A depicts indels detected byT7E1 analysis of HBG PCR product amplified from gDNA extracted from human mPB CD34⁺ cells treated with HBG RNPs (D10A paired nickases). For a subset of samples, cells also received ssODN encoding the 13 nt deletion plus silent SNPs to monitor for HDR (ssODN). FIG. 7B depicts DNA sequencing analysis for select subset of samples shown in FIG. 7A. The indels were subdivided according to the type of indel (insertion, 13 nt deletion, or other deletion).

FIG. 8A depicts the indels at the HBG target site after electroporation of mPB CD34⁺ cells with the indicated pairs of gRNAs complexed in D10A nickase and WT RNP pairs. FIG. 8B depicts the large deletion events (e.g. deletion of HBG2) after electroporation of mPB CD34⁺ cells with the indicated pairs of gRNAs complexed in D10A nickase and WT RNPs. FIG. 8C depicts DNA sequencing analysis and the subtypes of events (insertions, deletions) detected in gDNA from mPB CD34⁺ cells treated with paired D10A nickase pairs. FIG. 8D depicts DNA sequencing analysis and the subtypes of events (insertions, deletions) detected in gDNA from mPB CD34⁺ cells treated with paired WT RNP pairs.

FIG. 9 depicts the summary of HbF protein and mRNA expression in the progeny of mPB CD34⁺ cells treated with paired RNPs targeting HBG, for the experiments shown in FIGS. 7 and 8. HbF protein (by HPLC analysis) and HbF mRNA expression (ddPCR analysis) were evaluated in erythroid progeny of RNP treated human mPB CD34⁺ cells (background levels of HbF detected in donor matched untreated controls were subtracted from the levels detected in progeny of RNP treated CD34⁺ cells).

FIG. 10 depicts the indel frequencies and ex vivo and in vivo short-term hematopoietic potential of CD34⁺ cells after treatment with different concentrations (0, 2.5, 3.7 μM) of paired D10A nickase RNPs (SpA+Sp85). Indels were evaluated by T7E1 analysis (FIG. 10A) and by Illumina sequencing analysis (insertions and deletions, FIG. 10B). FIG. 10C depicts the % of HbF protein detected by HPLC analysis (% HbF=100% x HbF/(HbF+HbA). FIG. 10D depicts the hematopoietic activity of the RNP treated and donor matched untreated control CD34⁺ cells in colony forming cell (CFC) assays. CFCs shown are per thousand CD34⁺ cells plated. FIG. 10E depicts human blood CD45⁺ cell reconstitution of the peripheral blood in immunodeficient mice (NSG) 1 month after transplantation with donor matched human mPB CD34⁺ that were either untreated (0 μM), or treated with one of two doses (2.5 and 3.75 μM) of D10A RNP and paired gRNAs. FIG. 10F depicts human blood CD45⁺ cell reconstitution of the peripheral blood in immunodeficient mice (NSG) 2 months after transplantation. FIGS. 10G and 10H depict the lineage distributions following human CD45⁺ blood cell reconstitution of NSG mice at 1 month (FIG. 10G) and 2 months (FIG. 10H).

FIG. 11A correlates HbF levels as assayed by HPLC and indel frequency as assessed by T7E1 analysis for two D10A nickase RNP pairs (SP37+SPB and SP37+SPA) delivered at the indicated concentrations to mPB CD34⁺ cells. HbF levels were analyzed in erythroid progeny (day 18) of edited CD34⁺ cells. HbF protein detected in donor-matched untreated controls were subtracted from edited samples. FIG. 11B depicts indel rates overlaid on hematopoietic colony forming cell (CFC) activity associated with CD34⁺ cells treated with the indicated D10A nickase pairs or untreated controls. FIG. 11C depicts human CD45⁺ blood cell reconstitution of immunodeficient NSG mice one month after transplantation of mPB CD34⁺ cells treated with indicated D10 RNP nickase pairs at the concentrations given or donor matched untreated controls. FIG. 11D depicts the human blood lineage distribution detected in the human CD45⁺ fraction in mouse peripheral blood one month post-transplant.

FIG. 12 depicts a target site for de-repression of HbF, the GATA1 motif of the +58 DNase I hypersensitive site (DHS) erythroid specific enhancer of BCL11A (BCL11Ae) (genomic coordinates: chr2: 60,495,265 to 60,495,270).

FIG. 13A depicts the percentage of indels detected by T7E1 endonuclease analysis of BCL11A PCR products amplified from gDNA extracted from CB CD34⁺ cells treated with the indicated RNP+/−ssODN or donor matched untreated control cells. Data shown represent the mean of three 3 separate donors/experiments. FIG. 13B depicts indels detected by T7E1 endonuclease analysis of BCL11A PCR products amplified from gDNA extracted from CB CD34+ cells treated with the indicated WT RNP (single gRNA targeting the BCL11A erythroid enhancer complexed to WT S. pyogenes Cas9 having both RuvC and HNH activity) or paired nickase RNP (paired gRNAs targeting the BCL11A erythroid enhancer complexed to S. pyogenes Cas9 nickases sharing the same HNH single stranded cutting activity (e.g. D10A), as well as the hematopoietic activity of cells in each condition.

FIG. 14A depicts the editing frequency of BCL11Ae (using single gRNA approach targeting the GATA1 motif) in adult human BM CD34⁺ cells. FIG. 14B depicts the monoallelic and bialleleic editing detected in hematopoietic colonies (GEMMs, clonal progeny of BCL11Ae RNP treated CD34+ cells) as determined by DNA sequencing analysis. FIG. 14C depicts the kinetics of erythroblast maturation (enucleation as determined by DRAQ5⁻ cells detected by flow cytometry analysis). FIG. 14D depicts the acquisition of erythroid phenotype (Glycophorin A⁺ cells) in differentiated control and RNP-treated BM CD34⁺ cells, while FIG. 14E shows the fold increase in HbF⁺ cells as determined by flow cytometry analysis relative to HbF+ cells in untreated donor matched control samples.

FIG. 15 depicts gene editing of BCL11Ae in adult human mPB CD34+ cells and induction of fetal hemoglobin in erythroid progeny of RNP and ssODN treated cells after electroporation of mPB CD34⁺ cells with BCL11Ae RNP+nonspecific ssODN. FIG. 15A depicts the percentage of indels detected by T7E1 analysis of HBG2 PCR product amplified from gDNA extracted from mPB CD34⁺ cells treated with the BCL11Ae RNP and nonspecific ssODN or donor matched untreated control cells. FIG. 15B depicts the fold change in HBG mRNA expression in day 10 erythroblasts that were differentiated from BCL11Ae RNP treated and untreated donor matched control mPB CD34⁺ cells (mRNA levels are normalized to GAPDH and calibrated to the levels detected in untreated controls on the corresponding days of differentiation). FIG. 15C depicts the percentage of Glycophorin A expressed over the time course of erythroid differentiation of mPB CD34⁺ cells+/−treatment with BCL11Ae RNP and nonspecific ssODN, as determined by flow cytometry analysis at the indicated time points and for the indicated samples.

FIGS. 16A-B depict gene editing of the HBG promoter in and viability of adult human CD34⁺ cells from mPB electroporated with Cas9 complexed with Sp37 gRNA (targeting domain set forth in SEQ ID NO:933), which was in vitro transcribed (Sp37 IVT) or chemically synthesized (Sp37 Synthetic). FIG. 16A depicts the percentage of indels detected by sequencing the HBG PCR product amplified from adult human CD34⁺ cells from mPB electroporated with Cas9 complexed with Sp37 IVT or Sp37 Synthetic gRNAs. Next generation sequencing (NGS) (co-amplification and sequencing of HBG1 and HBG2 PCR product) was used to detect the indels. FIG. 16B depicts the percentage viability of the adult human mPB CD34⁺ cells 48 hours after electroporation with Cas9 complexed with Sp37 IVT or Sp37 Synthetic gRNAs.

FIG. 17 depicts gene editing of the HBG promoter in adult human CD34⁺ cells from mPB electroporated with Cas9 complexed with Sp35 gRNA (SEQ ID NO:970) and modified Sp35 gRNAs as disclosed in Table 10. The percentage of indels at the HBG promoter was determined by sequencing the HBG PCR product amplified from gDNA extracted post-electroporation with RNP.

FIGS. 18A-C depict gene editing of the HBG promoter in and viability of adult human CD34⁺ cells from mPB electroporated with ssODN “13 nt − strand” (i.e., OLI16412 (ssODN encoding the 13 nt deletion, negative (−) strand) or “13 nt+ strand” (i.e., OLI16414 (ssODN encoding the 13 nt deletion, positive (+) strand) (Table 11) and RNP targeting HBG containing Sp35 gRNA (SEQ ID NO:970) (OLI7066-sp35-RNP). FIG. 18A depicts the percentage of indels detected by sequencing the HBG PCR product from genomic DNA extracted at 72 hours post-electroporation. The grey portion of the bar represents the 13 nt indel deletion, whereas the white portion of the bar represents other indels. FIG. 18B depicts the percent viability of cells 48 hours post-electroporation. FIG. 18C depicts the percentage of relative expression levels of gamma-globin chains over total beta-like globin chains (gamma chains/[gamma chains+beta chain]) detected by ultra-performance liquid chromatography (UPLC).

FIG. 19 depicts human chimerism and lineage reconstitution 16 weeks after infusion of mPB CD34+ cells into nonirradiated NOD,B6.SCID Il2rγ−/− Kit(W41/W41) (NBSGW) mice. mPB CD34+ cells were electroporated with no RNP or with 8 μM of Sp35 RNP (comprising Sp35 gRNA (SEQ ID NO:339, Table 7) complexed with S. Pyogenes wildtype Cas9). Human chimerism (calculated as percentage of hCD45+ cells in total white blood cells in BM), erythroid reconstitution (calculated as percentage of CD235a+ cells in total BM cells, normalized to human chimerism level), monocyte reconstitution (calculated as percentage of CD14+ cells in hCD45+ population), neutrophil reconstitution (calculated as CD15+ cells in hCD45+ population), B-cell reconstitution (calculated as CD19+ cells in hCD45+ population), and HSPC reconstitution (calculated as CD34+ cells in hCD45+ population) was determined. Results from cells treated with Sp35 RNP (white bar) and without RNP (black bar) are shown.

FIGS. 20A-C depicts editing as determined by NGS for engrafted total human cells or flow-sorted CD235a+erythroid cells extracted from BM of nonirradiated NBSGW mice 16 weeks after infusion of modified mPB CD34+ cells. mPB CD34+ cells were electroporated with no RNP or with 8 μM of Sp35 RNP (comprising Sp35 gRNA (SEQ ID NO:339, Table 7) complexed with S. Pyogenes wildtype Cas9). FIG. 20A depicts indels in total unfractionated BM (black bar) and in erythroid cells (white bar). FIG. 20B depicts indels in erythroid cells (white bar) normalized to indels in corresponding BM (black bar). FIG. 20C depicts non-productive indels, defined as deletions that disrupt less than 3 nt of the distal CCAAT box, in erythroid cells (white bar) normalized to non-productive indels in corresponding BM (black bar).

FIG. 21 depicts apoptosis status of cultured erythroid cells from BM of nonirradiated NBSGW mice 16 weeks after infusion of modified mPB CD34+ cells. mPB CD34+ cells were electroporated with no RNP or with 8 μM of Sp35 RNP (comprising Sp35 gRNA (SEQ ID NO:339, Table 7) complexed with S. Pyogenes wildtype Cas9). Caspase-3 and caspase-7 activity, a marker for apoptotic cells, is shown for cells treated with no RNP (black bar) and cells treated with Sp35 RNP (white bar).

FIGS. 22A-B depict long term HbF induction by Sp35 RNP edited erythroid cells. mPB CD34+ cells were electroporated with no RNP or with Sp35 RNP (comprising Sp35 gRNA (SEQ ID NO: 339, Table 7) complexed with S. Pyogenes wildtype Cas9) and infused into NBSGW mice. FIG. 22A depicts HbF expression, defined as gamma/beta-like chains, by CD235a+erythroid cells sorted from BM of mice 16 weeks after infusion of mPB CD34+ cells treated with no RNP (black bar) or with Sp35 RNP (white bar). FIG. 22B depicts HbF expression by ex-vivo derived erythroid progeny of engrafted human cells from BM of mice 16 weeks after infusion of mPB CD34+ cells treated with no RNP (black bar) or with Sp35 RNP (white bar).

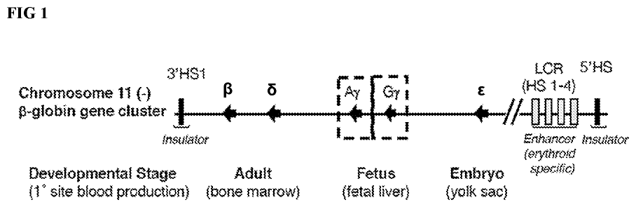

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai 2016 (incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a subject, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g. suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The notations “c. −114 to −102 region,” “13 nt target region” and the like refer to a sequence that is 5′ of the transcription start site (TSS) of the HBG1 and/or HBG2 gene. The HBG1 c. −114 to −102 region is exemplified in SEQ ID NO:902 (HBG1) at positions 2824-2836 and the HBG2 c. −114 to −102 region is exemplified in SEQ ID NO:903 (HBG2) at positions 2748-2760. The term “13 nt deletion” and the like refer to deletions of the 13 nt target region.

The term “proximal HBG1/2 promoter target sequence” denotes the region within 50, 100, 200, 300, 400, or 500 bp of a proximal HBG1/2 promoter sequence including the 13 nt target region. Alterations by genome editing systems according to this disclosure facilitate (e.g. cause, promote or tend to increase the likelihood of) upregulation of HbF production in erythroid progeny.

The term “GATA1 binding motif in BCL11Ae” refers to the sequence that is the GATA1 binding motif in the erythroid specific enhancer of BCL1 IA (BCL11Ae) that is in the +58 DNase I hypersensitive site (DHS) region of intron 2 of the BCL11A gene. The genomic coordinates for the GATA1 binding motif in BCL11Ae are chr2: 60,495,265 to 60,495,270. The +58 DHS site comprises a 115 base pair (bp) sequence as set forth in SEQ ID NO:968. The +58 DHS site sequence, including ˜500 bp upstream and −200 bp downstream is set forth in SEQ ID NO:969.

Where ranges are provided herein, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

### Overview

The various embodiments of this disclosure generally relate to genome editing systems configured to introduce alterations (e.g., a deletion or insertion, or other mutation) into chromosomal DNA that enhance transcription of the HBG1 and/or HBG2 genes, which encode the Aγ and Gγ subunits of hemoglobin, respectively. Exemplary mutations are made in or around the 13 nt target region of HBG1 and/or HBG2.

Fetal hemoglobin (HbF) expression can also be induced through targeted disruption of the erythroid cell specific expression of a transcriptional repressor, BCL11A, which encodes a repressor that silences HBG1 and HBG2 (Canvers 2015). Another gene editing strategy disclosed herein is to increase HbF expression by targeting disruption the of the erythroid specific enhancer of BCL11A (BCL11Ae) (also discussed in commonly-assigned International Patent Publication No. WO 2015/148860 by Friedland et al. (“Friedland,” incorporated by reference herein in its entirety). In certain embodiments, the region of BCL11Ae targeted for disruption may be the GATA1 binding motif in BCL11Ae. In certain embodiments, genome editing systems disclosed herein may be used to introduce alterations into the GATA1 binding motif in BCL11Ae and the 13 nt target region of HBG1 and/or HBG2.

The genome editing systems of this disclosure can include an RNA-guided nuclease such as Cas9 or Cpf1 and one or more gRNAs having a targeting domain that is complementary to a sequence in or near the target region, and optionally one or more of a DNA donor template that encodes a specific mutation (such as a deletion or insertion) in or near the target region, and/or an agent that enhances the efficiency with which such mutations are generated including, without limitation, a random oligonucleotide, a small molecule agonist or antagonist of a gene product involved in DNA repair or a DNA damage response, or a peptide agent.

A variety of approaches to the introduction of mutations into the 13 nt target region, proximal HBG1/2 promoter target sequence, and/or the GATA1 binding motif in BCL11Ae may be employed in the embodiments of the present disclosure. In one approach, a single alteration, such as a double-strand break, is made within the 13 nt target region, proximal HBG1/2 promoter target sequence, and/or the GATA1 binding motif in BCL11Ae, and is repaired in a way that disrupts the function of the region, for example by the formation of an indel or by the incorporation of a donor template sequence that encodes the deletion of the region. In a second approach, two or more alterations are made on either side of the region, resulting in the deletion of the intervening sequence, including the 13 nt target region and/or the GATA1 binding motif in BCL11Ae.

The treatment of hemoglobinopathies by gene therapy and/or genome editing is complicated by the fact that the cells that are phenotypically affected by the disease, erythrocytes or RBCs, are enucleated, and do not contain genetic material encoding either the aberrant hemoglobin protein (Hb) subunits nor the Aγ or Gγ subunits targeted in the exemplary genome editing approaches described above. This complication is addressed, in certain embodiments of this disclosure, by the alteration of cells that are competent to differentiate into, or otherwise give rise to, erythrocytes. Cells within the erythroid lineage that are altered according to various embodiments of this disclosure include, without limitation, hematopoietic stem and progenitor cells (HSCs), erythroblasts (including basophilic, polychromatic and/or orthochromatic erythroblasts), proerythroblasts, polychromatic erythrocytes or reticulocytes, embryonic stem (ES) cells, and/or induced pluripotent stem (iPSC) cells. These cells may be altered in situ (e.g. within a tissue of a subject) or ex vivo. Implementations of genome editing systems for in situ and ex vivo alteration of cells is described under the heading “Implementation of genome editing systems: delivery, formulations, and routes of administration” below.

In certain embodiments, alterations that result in induction of Aγ and/or Gγ expression are obtained through the use of a genome editing system comprising an RNA-guided nuclease and at least one gRNA having a targeting domain complementary to a sequence within the 13 nt target region of HBG1 and/or HBG2 or proximate thereto (e.g., within 10, 20, 30, 40, or 50, 100, 200, 300, 400 or 500 bases of the 13 nt target region). As is discussed in greater detail below, the RNA-guided nuclease and gRNA form a complex that is capable of associating with and altering the 13 nt target region or a region proximate thereto. Examples of suitable targeting domains directed to the 13 nt target region of HBG1 and/or HBG2 or proximate thereto for use in the embodiments disclosed herein include, without limitation, those set forth in SEQ ID NOs:251-901, 940-942. In certain embodiments, gRNAs targeting the 13 nt target region of HBG1 and/or HBG2 or proximate thereto may include modifications including, without limitation, addition of one or more PhTx or 2′-O-methyl groups. The modifications may be at or near the 5′ end or 3′ end of the gRNA or at or near the 5′ and 3′ ends of the gRNA. In certain embodiments, the gRNAs may be in vitro transcribed gRNAs or chemically synthesized gRNAs. Examples of suitable gRNAs directed to the 13 nt target region of HBG1 and/or HBG2 for use in the embodiments disclosed herein include, without limitation, those set forth in SEQ ID NOs:970-979.

In certain embodiments, alterations that result in induction of HbF expression are obtained through the use of a genome editing system comprising an RNA-guided nuclease and at least one gRNA having a targeting domain complementary to a sequence within the GATA1 binding motif in BCL11Ae or proximate thereto (e.g., within 10, 20, 30, 40, or 50, 100, 200, 300, 400 or 500 bases of the GATA1 binding motif in BCL11Ae). In certain embodiments, the RNA-guided nuclease and gRNA form a complex that is capable of associating with and altering the GATA1 binding motif in BCL11Ae. Examples of suitable targeting domains directed to the GATA1 binding motif in BCL11Ae for use in the embodiments disclosed herein include, without limitation, those set forth in SEQ ID NOs:952-955.

The genome editing system can be implemented in a variety of ways, as is discussed below in detail. As an example, a genome editing system of this disclosure can be implemented as a ribonucleoprotein complex or a plurality of complexes in which multiple gRNAs are used. This ribonucleoprotein complex can be introduced into a target cell using art-known methods, including electroporation, as described in commonly-assigned International Patent Publication No. WO 2016/182959 by Jennifer Gori (“Gori,” incorporated by reference herein in its entirety).

The ribonucleoprotein complexes within these compositions are introduced into target cells by art-known methods, including without limitation electroporation (e.g. using the Nucleofection™ technology commercialized by Lonza, Basel, Switzerland or similar technologies commercialized by, for example, Maxcyte Inc. Gaithersburg, Md.) and lipofection (e.g. using Lipofectamine™ reagent commercialized by Thermo Fisher Scientific, Waltham Mass.). Alternatively, or additionally, ribonucleoprotein complexes are formed within the target cells themselves following introduction of nucleic acids encoding the RNA-guided nuclease and/or gRNA. These and other delivery modalities are described in general terms below and in Gori.

Cells that have been altered ex vivo according to this disclosure can be manipulated (e.g. expanded, passaged, frozen, differentiated, de-differentiated, transduced with a transgene, etc.) prior to their delivery to a subject. The cells are, variously, delivered to a subject from which they are obtained (in an “autologous” transplant), or to a recipient who is immunologically distinct from a donor of the cells (in an “allogeneic” transplant).

In some cases, an autologous transplant includes the steps of obtaining, from the subject, a plurality of cells, either circulating in peripheral blood, or within the marrow or other tissue (e.g. spleen, skin, etc.), and manipulating those cells to enrich for cells in the erythroid lineage (e.g. by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system targeting the 13 nt target region, proximal HBG1/2 promoter target sequence, and/or the GATA1 binding motif in BCL11Ae. The genome editing system can be implemented or delivered to the cells in any suitable format, including as a ribonucleoprotein complex, as separated protein and nucleic acid components, and/or as nucleic acids encoding the components of the genome editing system.

In certain embodiments, CD34+ hematopoietic stem and progenitor cells (HSPCs) that have been edited using the genome editing methods disclosed herein may be used for the treatment of a hemoglobinopathy in a subject in need thereof. In certain embodiments, the hemoglobinopathy may be severe sickle cell disease (SCD) or thalassemia, such as (3-thalassemia, δ-thalassemia, or β/δ-thalassemia. In certain embodiments, an exemplary protocol for treatment of a hemoglobinopathy may include harvesting CD34+ HSPCs from a subject in need thereof, ex vivo editing of the autologous CD34+ HSPCs using the genome editing methods disclosed herein, followed by reinfusion of the edited autologous CD34+ HSPCs into the subject. In certain embodiments, treatment with edited autologous CD34+ HSPCs may result in increased HbF induction.

Prior to harvesting CD34+HSPCs, in certain embodiments, a subject may discontinue treatment with hydroxyurea, if applicable, and receive blood transfusions to maintain sufficient hemoglobin (Hb) levels. In certain embodiments, a subject may be administered intravenous plerixafor (e.g., 0.24 mg/kg) to mobilize CD34+HSPCs from bone marrow into peripheral blood. In certain embodiments, a subject may undergo one or more leukapheresis cycles (e.g., approximately one month between cycles, with one cycle defined as two plerixafor-mobilized leukapheresis collections performed on consecutive days). In certain embodiments, the number of leukapheresis cycles performed for a subject may be the number required to achieve a dose of edited autologous CD34+HSPCs (e.g., >2×10⁶ cells/kg, >3×10⁶ cells/kg, >4×10⁶ cells/kg, >5×10⁶ cells/kg, 2×10⁶ cells/kg to 3×10⁶ cells/kg, 3×10⁶ cells/kg to 4×10⁶ cells/kg, 4×10⁶ cells/kg to 5×10⁶ cells/kg) to be reinfused back into the subject, along with a dose of unedited autologous CD34+HSPCs/kg for backup storage (e.g., >1.5×10⁶ cells/kg). In certain embodiments, the CD34+HSPCs harvested from the subject may be edited using any of the genome editing methods discussed herein. In certain embodiments, any one or more of the gRNAs and one or more of the RNA-guided nucleases disclosed herein may be used in the genome editing methods.

In certain embodiments, the treatment may include an autologous stem cell transplant. In certain embodiments, a subject may undergo myeloablative conditioning with busulfan conditioning (e.g., dose-adjusted based on first-dose pharmacokinetic analysis, with a test dose of 1 mg/kg). In certain embodiments, conditioning may occur for four consecutive days. In certain embodiments, following a three-day busulfan washout period, edited autologous CD34+ HSPCs (e.g., >2×10⁶ cells/kg, >3×10⁶ cells/kg, >4×10⁶ cells/kg, ≥5×10⁶ cells/kg, 2×10⁶ cells/kg to 3×10⁶ cells/kg, 3×10⁶ cells/kg to 4×10⁶ cells/kg, 4×10⁶ cells/kg to 5×10⁶ cells/kg) may be reinfused into the subject (e.g., into peripheral blood). In certain embodiments, the edited autologous CD34+HSPCs may be manufactured and cryopreserved for a particular subject. In certain embodiments, a subject may attain neutrophil engraftment following a sequential myeloablative conditioning regimen and infusion of edited autologous CD34+ cells. Neutrophil engraftment may be defined as three consecutive measurements of ANC>0.5×10⁹/L.

However it is implemented, a genome editing system may include, or may be co-delivered with, one or more factors that improve the viability of the cells during and after editing, including without limitation an aryl hydrocarbon receptor antagonist such as StemRegenin-1 (SR1), UM171, LGC0006, alpha-napthoflavone, and CH-223191, and/or an innate immune response antagonist such as cyclosporin A, dexamethasone, reservatrol, a MyD88 inhibitory peptide, an RNAi agent targeting Myd88, a B18R recombinant protein, a glucocorticoid, OxPAPC, a TLR antagonist, rapamycin, BX795, and a RLR shRNA. These and other factors that improve the viability of the cells during and after editing are described in Gori, under the heading “I. Optimization of Stem Cells” from page 36 through page 61, which is incorporated by reference herein.

The cells, following delivery of the genome editing system, are optionally manipulated e.g. to enrich for HSCs and/or cells in the erythroid lineage and/or for edited cells, to expand them, freeze/thaw, or otherwise prepare the cells for return to the subject. The edited cells are then returned to the subject, for instance in the circulatory system by means of intravenous delivery or delivery or into a solid tissue such as bone marrow.

Functionally, alteration of the 13 nt target region, proximal HBG1/2 promoter target sequence, and/or the GATA1 binding motif in BCL11Ae using the compositions, methods and genome editing systems of this disclosure results in significant induction, among hemoglobin-expressing cells, of Aγ and/or Gγ subunits (referred to interchangeably as HbF expression), e.g. at least 5%, 10%, 15%, 20%, 25%, 30%, 35%, 40%, 45%, 50% or greater induction of Aγ and/or Gγ subunit expression relative to unmodified controls. This induction of protein expression is generally the result of alteration of the 13 nt target region, proximal HBG1/2 promoter target sequence, and/or the GATA1 binding motif in BCL11Ae (expressed, e.g. in terms of the percentage of total genomes comprising indel mutations within the plurality of cells) in some or all of the plurality of cells that are treated, e.g. at least 5%, 10%, 15%, 20%, 25%, 30%, 35%, 40%, 45%, 50% of the plurality of cells comprise at least one allele comprising a sequence alteration, including, without limitation, an indel, insertion, or deletion in or near the 13 nt target region, proximal HBG1/2 promoter target sequence, and/or the GATA1 binding motif in BCL11Ae.

The functional effects of alterations caused or facilitated by the genome editing systems and methods of the present disclosure can be assessed in any number of suitable ways. For example, the effects of alterations on expression of fetal hemoglobin can be assessed at the protein or mRNA level. Expression of HBG1 and HBG2 mRNA can be assessed by digital droplet PCR (ddPCR), which is performed on cDNA samples obtained by reverse transcription of mRNA harvested from treated or untreated samples. Primers for HBG1, HBG2, HBB, and/or HBA may be used individually or multiplexed using methods known in the art. For example, ddPCR analysis of samples may be conducted using the QX200™ ddPCR system commercialized by Bio Rad (Hercules, Calif.), and associated protocols published by BioRad. Fetal hemoglobin protein may be assessed by high pressure liquid chromatography (HPLC), for example, according to the methods discussed on pp. 143-44 in Chang 2017, incorporated by reference herein, or fast protein liquid chromatography (FPLC), using ion-exchange and/or reverse phase columns to resolve HbF, HbB and HbA and/or Aγ and Gγ globin chains as is known in the art.

It should be noted that the rate at which the 13 nt target region, proximal HBG1/2 promoter target sequence, and/or the GATA1 binding motif in BCL11Ae is altered in the target cells can be modified by the use of optional genome editing system components such as oligonucleotide donor templates. Donor template design is described in general terms below under the heading “Donor template design.” Donor templates for use in targeting the 13 nt target region may include, without limitation, donor templates encoding alterations (e.g., deletions) of HBG1 c. −114 to −102 (corresponding to nucleotides 2824-2836 of SEQ ID NO: 902), HBG1 c. −225 to −222 (corresponding to nucleotides 2716-2719 of SEQ ID NO:902)), and/or HBG2 c. −114 to −102 (corresponding to nucleotides 2748-2760 of SEQ ID NO:903). Exemplary 5′ and 3′ homology arms, and exemplary full-length donor templates encoding deletions such as c. −114 to −102 are also presented below (SEQ ID NOS: 904-909, 990-995). Donor templates used herein may be non-specific templates that are non-homologous to regions of DNA within or near the target sequence. In certain embodiments, donor templates for use in targeting the 13 nt target region may include, without limitation, non-target specific templates that are nonhomologous to regions of DNA within or near the 13 nt target region. For example, a non-specific donor template for use in targeting the 13 nt target region may be non-homologous to the regions of DNA within or near the 13 nt target region and may comprise a donor template encoding the deletion of HBG1 c. −225 to −222 (corresponding to nucleotides 2716-2719 of SEQ ID NO:902). In certain embodiments, donor templates for use in targeting the GATA1 binding motif in BCL11Ae may include, without limitation, non-target specific templates that are nonhomologous to regions of DNA within or near GATA1 binding motif in BCL11Ae target sequence. Other donor templates for use in targeting BCL11Ae may include, without limitation, donor templates including alterations (e.g., deletions) of BCL11Ae, including, without limitation, the GATA1 motif in BCL11Ae.

The embodiments described herein may be used in all classes of vertebrate including, but not limited to, primates, mice, rats, rabbits, pigs, dogs, and cats.

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova 2011, incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as, without limitation, a lipid or polymer micro- or nanoparticle, micelle, or liposome. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus (see section below under the heading “Implementation of genome editing systems: delivery, formulations, and routes of administration”); and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder,” incorporated by reference herein), describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, International Patent Publication No. WO 2016/073990 by Cotta-Ramusino et al. (“Cotta-Ramusino,” incorporated by reference herein), describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, International Patent Publication No. WO 2015/070083 by Palestrant et al. (incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

As disclosed herein, in certain embodiments, genome editing systems may comprise multiple gRNAs that may be used to introduce mutations into the GATA1 binding motif in BCL11Ae or the 13 nt target region of HBG1 and/or HBG2. In certain embodiments, genome editing systems disclosed herein may comprise multiple gRNAs used to introduce mutations into the GATA1 binding motif in BCL11Ae and the 13 nt target region of HBG1 and/or HBG2.

Genome editing systems can, in some instances, form double strand breaks that are repaired by cellular DNA double-strand break mechanisms such as NHEJ or HDR. These mechanisms are described throughout the literature (see, e.g., Davis & Maizels 2014 (describing Alt-HDR); Frit 2014 (describing Alt-NHEJ); Iyama & Wilson 2013 (describing canonical HDR and NHEJ pathways generally)).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g. fused to) a cytidine deaminase functional domain, and may operate by generating targeted C-to-A substitutions. Exemplary nuclease/deaminase fusions are described in Komor 2016, which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner 2014 (incorporated herein by reference) and Cotta-Ramusino. Examples of modular and unimolecular gRNAs that may be used according to the embodiments herein include, without limitation, the sequences set forth in SEQ ID NOs:29-31 and 38-51. Examples of gRNA proximal and tail domains that may be used according to the embodiments herein include, without limitation, the sequences set forth in SEQ ID NOs:32-37.

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g. GAAA) “tetraloop” or “linker” sequence bridging complementary regions of the crRNA (at its 3′ end) and the tracrRNA (at its 5′ end) (Mali 2013; Jiang 2013; Jinek 2012; all incorporated by reference herein).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu 2013, incorporated by reference herein), “complementarity regions” (Cotta-Ramusino), “spacers” (Briner 2014) and generically as “crRNAs” (Jiang 2013). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes (Nishimasu 2014; Nishimasu 2015; both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner 2014.

While the foregoing description has focused on gRNAs for use with Cas9, it should be appreciated that other RNA-guided nucleases exist which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche 2015a, incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously (see, e.g., Mali 2013; Hsu 2013; Fu 2014; Heigwer 2014; Bae 2014; Xiao 2014; all incorporated by reference herein). As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

With respect to selection of gRNA targeting domain sequences directed to HBG1/2 target sites (e.g. the 13 nt target region), an in-silico gRNA target domain identification tool was utilized, and the hits were stratified into four tiers. For S. pyogenes, tier 1 targeting domains were selected based on (1) distance upstream or downstream from either end of the target site (i.e., HBG1/2 13 nt target region), specifically within 400 bp of either end of the target site, (2) a high level of orthogonality, and (3) the presence of 5′ G. Tier 2 targeting domains were selected based on (1) distance upstream or downstream from either end of the target site (i.e., HBG1/2 13 nt target region), specifically within 400 bp of either end of the target site, and (2) a high level of orthogonality. Tier 3 targeting domains were selected based on (1) distance upstream or downstream from either end of the target site (i.e., HBG1/2 13 nt target region), specifically within 400 bp of either end of the target site and (2) the presence of 5′ G. Tier 4 targeting domains were selected based on distance upstream or downstream from either end of the target site (i.e., HBG1/2 13 nt target region), specifically within 400 bp of either end of the target site.

For S. aureus, tier 1 targeting domains were selected based on (1) distance upstream or downstream from either end of the target site (i.e., HBG1/2 13 nt target region), specifically within 400 bp of either end of the target site, (2) a high level of orthogonality, (3) the presence of 5′ G, and (4) PAM having the sequence NNGRRT (SEQ ID NO:204). Tier 2 targeting domains were selected based on (1) distance upstream or downstream from either end of the target site (i.e., HBG1/2 13 nt target), specifically within 400 bp of either end of the target site, (2) a high level of orthogonality, and (3) PAM having the sequence NNGRRT (SEQ ID NO:204). Tier 3 targeting domains were selected based on (1) distance upstream or downstream from either end of the target site (i.e., HBG1/2 13 nt target region), specifically within 400 bp of either end of the target site, and (2) PAM having the sequence NNGRRT (SEQ ID NO:204). Tier 4 targeting domains were selected based on (1) distance upstream or downstream from either end of the target site (i.e., HBG1/2 13 nt target), specifically within 400 bp of either end of the target site, and (2) PAM having the sequence NNGRRV (SEQ ID NO:205).

Table 2, below, presents targeting domains for S. pyogenes and S. aureus gRNAs, broken out by (a) tier (1, 2, 3 or 4) and (b) HBG1 or HBG2.

TABLE 2
gRNA targeting domain sequences for HBG1/2 target sites

| | | HBG1 | HBG2 |
|---|---|---|---|
| S. pyogenes | Tier 1 | 251-256 | 760-764 |
| | Tier 2 | 257-274 | 765-781 |
| | Tier 3 | 275-300 | 275-281, 283-300 |
| | Tier 4 | 301-366 | 301-311, 313-342, 344- |
| | | | 348, 350-366, 782, 783 |
| S. aureus | Tier 1 | 367-376 | 784-791 |
| | Tier 2 | 343, 377-393 | 778, 792-803 |
| | Tier 3 | 357, 365, 394-461 | 357, 365, 394-461 |
| | Tier 4 | 252-254, 256, | 292, 295, 347, 348, 353, |
| | | 268, 272-274, | 360-362, 366, 462-468 |
| | | 292, 295, 347, | 476-481, 489-587, 601- |
| | | 348, 353, 360- | 607, 614-620, 640-666, |
| | | 362, 366, 598-759 | 674-679, 687-693, 708- |
| | | | 714, 733-753, 762-764, |
| | | | 775, 779-781, 804-901 |

In certain embodiments, the gRNAs described herein may be chemically synthesized. As shown in Example 8 below, chemically synthesized Sp37 gRNA (targeting domain set forth in SEQ ID NO:933), which targets the 13 nt deletion associated with HPFH, produced an increased amount of indels compared to in vitro transcribed Sp37 gRNA. Additional exemplary gRNAs that may be chemically synthesized are set forth in SEQ ID NO:970-979 (Table 10).

gRNAs may be designed to target the erythroid specific enhancer of BCL11A (BCL11Ae) to disrupt expression of a transcriptional repressor, BCL11A (described in Friedland). gRNAs were designed to target the GATA1 binding motif that is in the erythroid specific enhancer of BCL11A that is in the +58 DHS region of intron 2 (i.e., the GATA1 binding motif in BCL11Ae), where the +58 DHS enhancer region comprises the sequence set forth in SEQ ID NO:968. Targeting domain sequences of gRNAs that were designed to target disruption of the GATA1 binding motif in BCL11Ae, include, but are not limited to, the sequences set forth in SEQ ID NOs:952-955. Targeting domain sequences plus PAM (NGG) of gRNAs that were designed to target disruption of the GATA1 binding motif in BCL11Ae, include, but are not limited to, the sequences set forth in SEQ ID NOs:960-963.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)), as shown below:

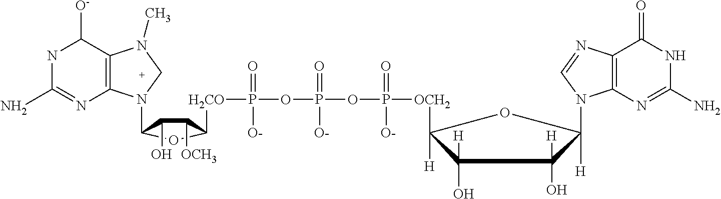

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

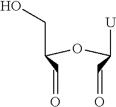

wherein “U” can be an unmodified or modified uridine.

The 3′ terminal U ribose can be modified with a 2′3′ cyclic phosphate as shown below:

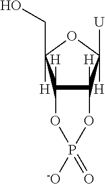

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphorothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

Examples of gRNAs that include modifications and that may be used according to the embodiments herein include, without limitation, gRNAs that target the 13 nt target region. In certain embodiments, gRNAs may contain one or more modifications. In certain embodiments, the modifications may be at or near the 5′end, 3′ end or both the 5′ and 3′ ends of the gRNA. In certain embodiments, modifications may include, without limitation, addition of one or more PhTx groups or one or more 2′-O-methyl groups. In certain embodiments, the Sp35 gRNA (SEQ ID NO:970) may contain modifications at or near the 5′end and/or 3′ ends. Exemplary gRNAs that include modifications of Sp35 gRNA, such as additions of one or more of PhTx groups and/or 2′-O-methyl groups are set forth in Table 10.

In certain embodiments, gRNAs used according to the embodiments herein may include truncated forms of a full length gRNA. In certain embodiments, truncated gRNAs may have one or more nucleotides removed from the 5′ or 3′ end of the gRNA compared with a full-length gRNA. In certain embodiments, truncated gRNAs have one or more nucleotides removed from the protospacer region of the gRNA. Examples of gRNAs that are truncated that may be used according to the embodiments herein include, without limitation, the truncated forms of the gRNA Sp35 (SEQ ID NO:970), such as the sequences set forth in SEQ ID NOs:976-978 (Table 10).

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g. full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease). Examples of PAMs that may be used according to the embodiments herein include, without limitation, the sequences set forth in SEQ ID NOs:199-205.

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above and in Ran & Hsu 2013, incorporated by reference herein), or that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat: anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity. Examples of polypeptide sequences encoding Cas9 RuvC-like and Cas9 HNH-like domains that may be used according to the embodiments herein are set forth in SEQ ID NOs:15-23, 52-123 (RuvC-like domains) and SEQ ID NOs:24-28, 124-198 (HNH-like domains).

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains). Examples of polypeptide sequences encoding Cas9 molecules that may be used according to the embodiments herein are set forth in SEQ ID NOs:1-2, 4-6, 12, 14.

### Cpf1

The crystal structure of Acidaminococcus sp. Cpf1 in complex with crRNA and a double-stranded (ds) DNA target including a TTTN PAM sequence has been solved by Yamano 2016 (incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, —II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, —II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran & Hsu 2013 and Yamano 2016, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand. On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver 2015a) and S. aureus (Kleinstiver 2015b. Kleinstiver et al. have also described modifications that improve the targeting fidelity of Cas9 (Kleinstiver 2016). Each of these references is incorporated by reference herein.

RNA-guided nucleases have been split into two or more parts, as described by Zetsche 2015b and by Fine 2015, both incorporated by reference herein.

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger 2014, which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Examples of nucleic acid sequences encoding Cas9 molecules that may be used according to the embodiments herein are set forth in SEQ ID NOs:3, 7-11, 13. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

### Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10×SYPRO Orange® (Life Technologies cat#S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat#S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson 2016 (incorporated by reference herein). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

Genome editing systems according to this disclosure may also be employed for multiplex gene editing to generate two or more DSBs, either in the same locus or in different loci. Any of the RNA-guided nucleases and gRNAs disclosed herein may be used in genome editing systems for multiplex gene editing. Strategies for editing that involve the formation of multiple DSBs, or SSBs, are described in, for instance, Cotta-Ramusino.

As disclosed herein, multiple gRNAs may be used in genome editing systems to introduce alterations (e.g., deletions, insertions) into the 13 nt target region of HBG1 and/or HBG2. In certain embodiments, one or more gRNAs comprising a targeting domain set forth in SEQ ID NOs:251-901, 940-942 may be used to introduce alterations in the 13 nt target region of HBG1 and/or HBG2. In certain embodiments, one or more gRNAs comprising a sequence set forth in SEQ ID NOs:970-979 may be used to introduce alterations in the 13 nt target region of HBG1 and/or HBG2. In other embodiments, multiple gRNAs may be used in genome editing systems to introduce alterations into the GATA1 binding motif in BCL11Ae. In certain embodiments, one or more gRNAs comprising a targeting domain set forth in SEQ ID NOs:952-955 may be used to introduce alterations in the GATA1 binding motif in BCL11Ae. Multiple gRNAs may also be used in genome editing systems to introduce alterations into the GATA1 binding motif in BCL11Ae and the 13 nt target region of HBG1 and/or HBG2. In certain embodiments, one or more gRNAs comprising a targeting domain set forth in SEQ ID NOs:952-955 may be used to introduce alterations in the GATA1 binding motif in BCL11Ae and one or more gRNAs comprising a targeting domain set forth in SEQ ID NOs:251-901, 940-942 and/or one or more gRNAs comprising a sequence set forth in SEQ ID NOs:970-979 may be used to introduce alterations in the 13 nt target region ofHBG1 and/or HBG2.

### Donor Template Design

Donor template design is described in detail in the literature, for instance in Cotta-Ramusino. DNA oligomer donor templates (oligodeoxynucleotides, ODNs, or template nucleic acids), which can be single stranded (ssODNs) or double-stranded (dsODNs), can be used to facilitate HDR-based repair of DSBs or to boost overall editing rate, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:
[5′ homology arm]-[replacement sequence]-[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson 2016, which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or then, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino, which is incorporated by reference.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

In certain embodiments, silent, non-pathogenic SNPs may be included in the ssODN donor template to allow for identification of a gene editing event.

In certain embodiments, a donor template may be a non-specific template that is non-homologous to regions of DNA within or near a target sequence to be cleaved. In certain embodiments, donor templates for use in targeting the GATA1 binding motif in BCL11Ae may include, without limitation, non-target specific templates that are nonhomologous to regions of DNA within or near the GATA1 binding motif in BCL11Ae. In certain embodiments, donor templates for use in targeting the 13 nt target region may include, without limitation, non-target specific templates that are nonhomologous to regions of DNA within or near the 13 nt target region.

A donor template or template nucleic acid, as that term is used herein, refers to a nucleic acid sequence which can be used in conjunction with an RNA nuclease molecule and one or more gRNA molecules to alter (e.g., delete, disrupt, or modify) a target DNA sequence. In certain embodiments, the template nucleic acid results in a 13 nt deletion at the 13 nt target region of HBG1 and/or HBG2. In certain embodiments, the alteration (e.g., deletion) may be selected from HBG1 13 del c. −114 to −102, HBG2 13 del c. −114 to −102, or a combination thereof. In certain embodiments, the 13 nt target region may be selected from HBG1 c. −114 to −102 (e.g., nucleotides 2824-2836 of SEQ ID NO:902 (HBG1)), and HBG2 c. −114 to −102 (e.g., nucleotides 2748-2760 of SEQ ID NO:903 (HBG2)), or a combination thereof. In certain embodiments, the template nucleic acid may be a positive strand or a negative strand.

For example, a template nucleic acid for introducing the 13 nt deletion at the 13 nt target region (HBG1 c. −114 to −102 (e.g., nucleotides 2824-2836 of SEQ ID NO:902 (HBG1)), HBG2 c. −114 to −102 (e.g., nucleotides 2748-2760 of SEQ ID NO:903 (HBG2)), or a combination thereof may comprise a 5′ homology arm, a replacement sequence, and a 3′ homology arm, where the replacement sequence is 0 nucleotides or 0 bp. In certain embodiments, the 5′ homology arm comprises about 200 nucleotides in length, e.g., at least 25, 50, 75, 100, 125, 150, 175, or 200 nucleotides in length. In certain embodiments, the 5′ homology arm comprises about 50 to 100 bp, e.g., 55 to 95, 60 to 90, 70 to 90, or 80 to 90 bp, homology 5′ of the 13 nt target region (e.g., HBG1 c. −114 to −102 (e.g., nucleotides 2824-2836 of SEQ ID NO:902 (HBG1)) or HBG2 c. −114 to −102 (e.g., nucleotides 2748-2760 of SEQ ID NO:903 (HBG2)). In certain embodiments, the 5′ homology arm comprises, consists essentially of, or consists of SEQ ID NO:904 (ssODN1 5′ homology arm), SEQ ID NO:907 (PhTx ssODN1 5′homology arm), SEQ ID NO:991 (01116414 5′ homology arm), or SEQ ID NO:994 (01116412 5′ homology arm). In certain embodiments, the 3′ homology arm comprises about 200 nucleotides in length, e.g., at least 25, 50, 75, 100, 125, 150, 175, or 200 nucleotides in length. In certain embodiments, the 3′ homology arm comprises about 50 to 100 bp, e.g., 55 to 95, 60 to 90, 70 to 90, or 80 to 90 bp, homology 3′ of the 13 nt target region (e.g., HBG1 c. −114 to −102 (e.g., nucleotides 2824-2836 of SEQ ID NO:902 (HBG1)) or HBG2 c. −114 to −102 (e.g., nucleotides 2748-2760 of SEQ ID NO:903 (HBG2)). In certain embodiments, the 3′ homology arm comprises, consists essentially of, or consists of SEQ ID NO:905 (ssODN1 3′ homology arm), SEQ ID NO:908 (PhTx ssODN1 3′homology arm), SEQ ID NO:992 (01116414 3′ homology arm), or SEQ ID NO:995 (01116412 3′ homology arm). In certain embodiments, the template nucleic acid comprises, consists essentially of, or consists of SEQ ID NO:906 (ssODN1), SEQ ID NO:909 (PhTx ssODN1), SEQ ID NO:990 (OLI16414), or SEQ ID NO:995 (OLI16412).

In certain embodiments, the template nucleic acid comprises one or more phosphorothioate (PhTx) modifications. In certain embodiments, the 5′ homology arm comprises one or more PhTx modifications. In certain embodiments, the 3′ homology arm comprises one or more PhTx modifications. In certain embodiments, the template nucleic acid comprises one or more PhTx modifications at or near the 5′ end and/or 3′ end.

In certain embodiments, the ssODNs for introducing the 13 nt deletion at the 13 nt target region described herein may be used in conjunction with an RNA nuclease and one or more gRNAs that target the 13 nt target region, for example, the gRNAs disclosed in Table 10. In certain embodiments, the gRNAs may be chemically synthesized gRNAs.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 3 and 4 set forth several, non-limiting examples of genome editing system implementations. Those of skill in the art will appreciate, however, that these listings are not comprehensive, and that other implementations are possible. With reference to Table 3 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 3
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described |
| | | | above plus a single-stranded or |
| | | | double stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both |
| | | | a gRNA and a donor template. |
| DNA | | | A DNA or DNA vector encoding |
| | | | an RNA-guided nuclease, a gRNA |
| | | | and a donor template. |
| DNA | DNA | [N/A] | Two separate DNAs, or two |
| | | | separate DNA vectors, encoding |
| | | | the RNA-guided nuclease and the |
| | | | gRNA, respectively. |
| DNA | DNA | DNA | Three separate DNAs, or three |
| | | | separate DNA vectors, encoding |
| | | | the RNA-guided nuclease, the |
| | | | gRNA and the donor template, |
| | | | respectively. |
| DNA | | [N/A] | A DNA or DNA vector encoding an |
| | | | RNA-guided nuclease and a gRNA |
| DNA | | DNA | A first DNA or DNA vector |
| | | | encoding an RNA-guided nuclease |
| | | | and a gRNA, and a second DNA or |
| | | | DNA vector encoding a donor |
| | | | template. |
| DNA | DNA | | A first DNA or DNA vector |
| | | | encoding an RNA-guided nuclease |
| | | | and second DNA or DNA vector |
| | | | encoding a gRNA and a donor |
| | | | template. |
| DNA | | | A first DNA or DNA vector |
| \| DNA \| | | | encoding an RNA-guided nuclease |
| | | | and a donor template, and a second |
| | | | DNA or DNA vector encoding a |
| | | | gRNA |
| DNA | | | A DNA or DNA vector encoding an |
| \| RNA \| | | | RNA-guided nuclease and a donor |
| | | | template, and a gRNA |
| RNA | | [N/A] | An RNA or RNA vector encoding |
| | | | an RNA-guided nuclease and |
| | | | comprising a gRNA |
| RNA | | DNA | An RNA or RNA vector encoding |
| | | | an RNA-guided nuclease and |
| | | | comprising a gRNA, and a DNA or |
| | | | DNA vector encoding a donor |
| | | | template. |

Table 4 summarizes various delivery methods for the components of genome editing systems, as described herein. Again, the listing is intended to be exemplary rather than limiting.

TABLE 4

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium Phosphate | | | | | and Proteins |
| transfection, cell compression or | | | | | |
| squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 4, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 4, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nonparticles can be suitable for use as delivery vehicles in certain embodiments of this disclosure. Exemplary lipids for use in nanoparticle formulations, and/or gene transfer are shown in Table 5, and Table 6 lists exemplary polymers for use in gene transfer and/or nanoparticle formulations.

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadeceny1-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

TABLE 6
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly(2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo. A protective, interactive, non-condensing (PINC) system may be used for delivery.

In vitro delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

### Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Multi-Modal or Differential Delivery of Components

Skilled artisans will appreciate, in view of the instant disclosure, that different components of genome editing systems disclosed herein can be delivered together or separately and simultaneously or non-simultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### EXAMPLES

The principles and embodiments described above are further illustrated by the non-limiting examples that follow:

### Example 1: Screening of S. pyogenes gRNAs Delivered to K562 Cells as Ribonucleoprotein Complexes for Use in Causing 13 nt Deletions in HBG1 and HBG2 Regulatory Regions

gRNAs targeting a 26 nt fragment spanning and including the 13 nucleotides at the 13 nt target region of HBG1 and HBG2 were designed by standard methods. After gRNAs were designed in silico and tiered, a subset of the gRNAs were selected and screened for activity and specificity in human K562 cells. The gRNAs selected for screening are set forth in Table 7. Briefly, gRNAs were in vitro transcribed and then complexed with S. pyogenes wildtype (Wt) Cas9 protein to form ribonucleoprotein complexes (RNPs). The gRNAs complexed to S. pyogenes Cas9 protein were modified sgRNAs ((e.g., 5′ ARCA capped and 3′ polyA (20A) tail; Table 7) and target the HBG1 and HBG2 regulatory regions. To allow for direct comparison of the activity of these RNPs in K562 cells and human CD34⁺ cells, RNPs were first delivered to K562 cells by electroporation (Amaxa Nucleofector).

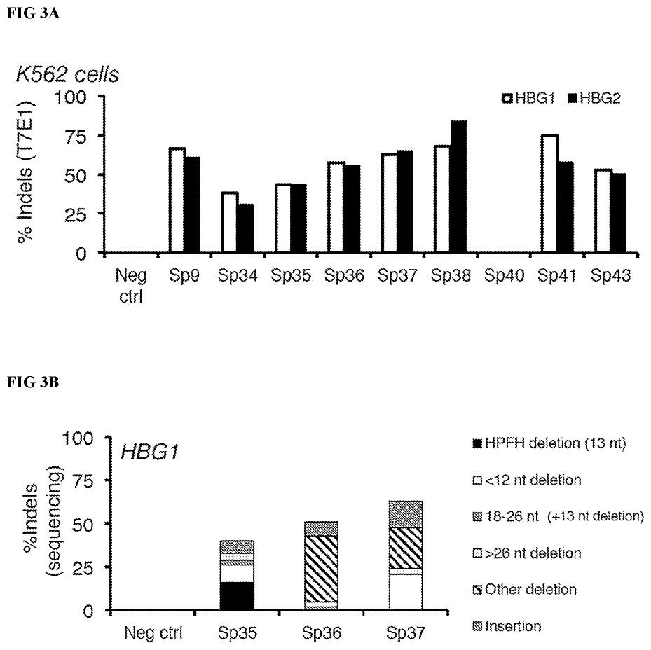

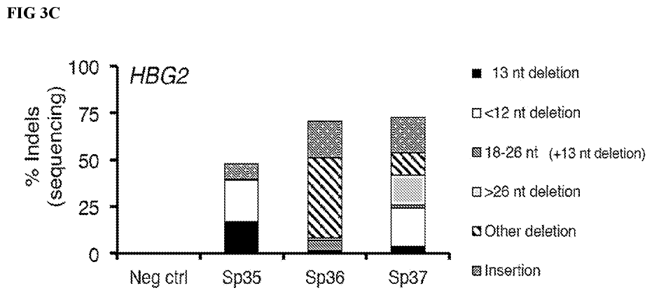

Three days after RNP electroporation, gDNA was extracted from K562 cells and then the HBG1 and HBG2 loci were PCR amplified from the gDNA. Gene editing was evaluated in the PCR products by T7E1 endonuclease assay analysis. Eight out of nine RNPs supported a high percentage of NHEJ. Sp37 RNP, the only gRNA shown to be active in human CD34⁺ cells (<10% editing in CD34⁺ cells) was highly active in K562 cells, with >60% indels detected at both HBG1 and HBG2 and eight cut in both the HBG1 and HBG2 targeted regions in the promoter sequences (FIG. 3A).

TABLE 7
Selected gRNAs for screening in K562 cells or CD34⁺ cells

| | | | | Targeting domain | |
|---|---|---|---|---|---|
| | Targeting | | Targeting domain | sequence plus | |
| gRNA | domain sequence | Targeting domain | sequence plus PAM | PAM (NGG) | |
| ID | (RNA) | sequence (DNA) | (NGG) (RNA) | (DNA) | Sense |
| Sp9 | GGCUAUUGGU | GGCTATTGGTCA | GGCUAUUGGUCA | GGCTATTGGTCA | Antisense |
| | CAAGGCA | AGGCA | AGGCAAGG | AGGCAAGG | |
| | (SEQ ID NO: 277) | (SEQ ID NO: 910) | (SEQ ID NO: 920) | (SEQ ID NO: 930) | |
| Sp36 | CAAGGCUAUU | CAAGGCTATTGG | CAAGGCUAUUGG | CAAGGCTATTGG | Antisense |
| | GGUCAAGGCA | TCAAGGCA | UCAAGGCAAGG | TCAAGGCAAGG | |
| | (SEQ ID NO: 338) | (SEQ ID NO: 911) | (SEQ ID NO: 921) | (SEQ ID NO: 931) | |
| Sp40 | UGCCUUGUCA | TGCCTTGTCAAG | UGCCUUGUCAAGG | TGCCTTGTCAAG | Antisense |
| | AGGCUAU | GCTAT | CUAUUGG | GCTATTGG | |
| | (SEQ ID NO: 327) | (SEQ ID NO: 912) | (SEQ ID NO: 922) | (SEQ ID NO: 932) | |
| Sp42 | GUUUGCCUUG | GTTTGCCTTGTC | GUUUGCCUUGUCA | GTTTGCCTTGTC | Antisense |
| | UCAAGGCUAU | AAGGCTAT | AGGCUAUUGG | AAGGCTATTGG | |
| | (SEQ ID NO: 299) | (SEQ ID NO: 913) | (SEQ ID NO: 923) | (SEQ ID NO: 933) | |
| Sp38 | GACCAAUAGC | GACCAATAGCCT | GACCAAUAGCCUU | GACCAATAGCCT | Sense |
| | CUUGACA | TGACA | GACAAGG | TGACAAGG | |
| | (SEQ ID NO: 276) | (SEQ ID NO: 914) | (SEQ ID NO: 924) | (SEQ ID NO: 934) | |
| Sp37 | CUUGACCAAU | CTTGACCAATAG | CUUGACCAAUAGC | CTTGACCAATAG | Sense |
| | AGCCUUGACA | CCTTGACA | CUUGACAAGG | CCTTGACAAGG | |
| | (SEQ ID NO: 333) | (SEQ ID NO: 915) | (SEQ ID NO: 925) | (SEQ ID NO: 935) | |
| Sp43 | GUCAAGGCUA | GTCAAGGCTATT | GUCAAGGCUAUU | GTCAAGGCTATT | Antisense |
| | UUGGUCA | GGTCA | GGUCAAGG | GGTCAAGG | |
| | (SEQ ID NO: 278) | (SEQ ID NO: 916) | (SEQ ID NO: 926) | (SEQ ID NO: 936) | |
| Sp35 | CUUGUCAAGG | CTTGTCAAGGCT | CUUGUCAAGGCUA | CTTGTCAAGGCT | Antisense |
| | CUAUUGGUCA | ATTGGTCA | UUGGUCAAGG | ATTGGTCAAGG | |
| | (SEQ ID NO: 339) | (SEQ ID NO: 917) | (SEQ ID NO: 927) | (SEQ ID NO: 937) | |
| Sp41 | UCAAGUUUGC | TCAAGTTTGCCT | UCAAGUUUGCCUU | TCAAGTTTGCCT | Antisense |
| | CUUGUCA | TGTCA | GUCAAGG | TGTCAAGG | |
| | (SEQ ID NO: 310) | (SEQ ID NO: 918) | (SEQ ID NO: 928) | (SEQ ID NO: 938) | |
| Sp34 | UGGUCAAGUU | TGGTCAAGTTTG | UGGUCAAGUUUG | TGGTCAAGTTTG | Antisense |
| | UGCCUUGUCA | CCTTGTCA | CCUUGUCAAGG | CCTTGTCAAGG | |
| | (SEQ ID NO: 340) | (SEQ ID NO: 919) | (SEQ ID NO: 929) | (SEQ ID NO: 939) | |
| Sp85 | AGUAUCCAGU | AGTATCCAGTGA | AGUAUCCAGUGA | AGTATCCAGTGA | Antisense |
| | GAGGCCA | GGCCA | GGCCAGGG | GGCCAGGG | |
| | (SEQ ID NO: 940) | (SEQ ID NO: 943) | (SEQ ID NO: 946) | (SEQ ID NO: 949) | |
| SpA | GGCAAGGCUG | GGCAAGGCTGG | GGCAAGGCUGGCC | GGCAAGGCTGG | Sense |
| | GCCAACCCAU | CCAACCCAT | AACCCAUGGG | CCAACCCATGGG | |
| | (SEQ ID NO: 941) | (SEQ ID NO: 944) | (SEQ ID NO: 947) | (SEQ ID NO: 950) | |
| SpB | UAUUUGCAUU | TATTTGCATTGA | UAUUUGCAUUGA | TATTTGCATTGA | Sense |
| | GAGAUAGUGU | GATAGTGT | GAUAGUGUGGG | GATAGTGTGGG | |
| | (SEQ ID NO: 942) | (SEQ ID NO: 945) | (SEQ ID NO: 948) | (SEQ ID NO: 951) | |

The HBG1 and HBG2 PCR products for the K562 cells that were targeted with the eight active sgRNAs were then analyzed by DNA sequencing analysis and scored for insertions and deletions detected. The deletions were subdivided into precise 13 nt deletions at the target site, 13 nt target site inclusive and proximal small deletions (18-26 nt), 12 nt deletions (i.e., partial deletion) of the 13 nt target site, >26 nt deletions that span a portion of the HPFH target site, and other deletions, e.g., deletions proximal to but outside the HPFH target site. Seven of the eight sgRNAs targeted deletion of the 13 nt (HPFH mutation induction) (FIG. 3B) for HBG1. At least five of the eight sgRNAs also supported targeted deletion of the 13 nt in HBG2 promoter region (FIG. 3C). Note that DNA sequence results for HBG2 in cells treated with HBG Sp34 sgRNA were not available. These data indicate that Cas9 and sgRNA support precise induction of the 13 nt deletions. FIGS. 3B-3C depict examples of the types of deletions observed in target sequences in HBG1.

### Example 2: Cas9 RNP Containing gRNA Targeting the 13 nt Deletion Mutation Supports Gene Editing in Human Hematopoietic Stem/Progenitor Cells

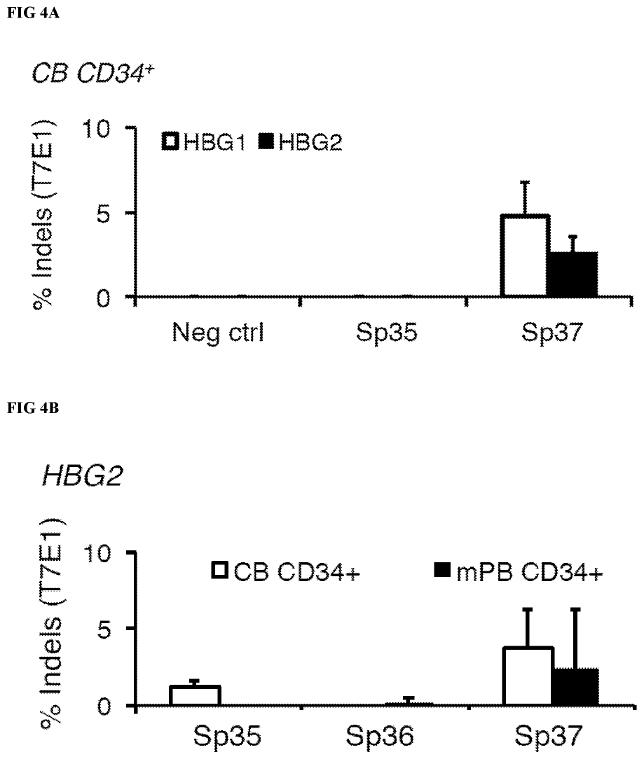

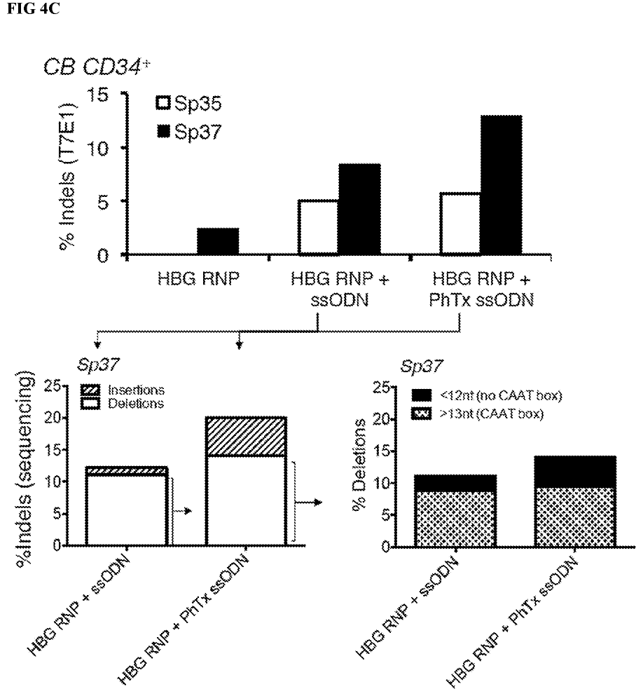

Of the RNPs containing different gRNAs tested in human cord blood (CB) CD34⁺ cells, only Sp37 resulted in detectable editing at the target site in the HBG1 and HBG2 promoters as determined by T7E1 analysis of indels in HBG1 and HBG2 specific PCR products amplified from gDNA extracted from electroporated CB CD34⁺ cells from a three cord blood donors (FIG. 4A). The average level of editing detected in cells electroporated with Cas9 protein complexed to Sp37 was 5±2% indels at HBG1 and 3±1% indels detected at HBG2 (3 separate experiments, and CB donors).

Next, three S. pyogenes gRNAs whose target sites are within the HBG promoter (Sp35, Sp36, Sp37) were complexed to wild-type S. pyogenes Cas9 protein to form ribonucleoprotein complexes. These HBG targeted RNPS were electroporated into CB CD34⁺ cells (n=3 donors) and adult mobilized peripheral blood (mPB) CD34⁺ cell donors (n=3 donors). Then the level of editing at the target site was analyzed by T7E1 endonuclease analysis of the HBG2 PCR products amplified from genomic DNA extracted from the samples approximately 3 days after Cas9 RNP delivery. Each of these RNPs supported only low level gene editing in both the CB and adult CD34⁺ cells across 3 donors and 3 separate experiments (FIG. 4B).

To increase gene editing and the occurrence of the 13 nt deletion at the target site, single strand deoxynucleotide donor repair templates (ssODNs) that encoded 87 nt and 89 nt of homology on each side of the targeted deletion site was generated. The ssODNs, either unmodified at the ends (i.e., ssODN1, SEQ ID NO:906, Table 8) or modified to contain phosphorothioates (PhTx) at the 5′ and 3′ ends (i.e., PhTx ssODN1, SEQ ID NO:909, Table 8). The ssODN was designed to ‘encode’ the 13 nt deletion with sequence homology arms engineered flanking this absent sequence to create a perfect deletion.

TABLE 8
Single strand deoxynucleotide donor repair templates (ssODN)

| | SEQ | |
|---|---|---|
| ssODN | ID NO | Sequence |
| ssODN1 | 904 | GGGTGCTTCCTTTTATTCTTCATCCCTAGCCAGCCGCCGGCCCCTG |
| 5′ homology arm | | GCCTCACTGGATACTCTAAGACTATTGGTCAAGTTTGCCTT |
| ssODN1 | 905 | GTCAAGGCAAGGCTGGCCAACCCATGGGTGGAGTTTAGCCAGGGACCG |
| 3′ homology arm | | TTTCAGACAGATATTTGCATTGAGATAGTGTGGGGAAGGGG |
| ssODN1 | 906 | GGGTGCTTCCTTTTATTCTTCATCCCTAGCCAGCCGCCGGCCCCTG |
| | | GCCTCACTGGATACTCTAAGACTATTGGTCAAGTTTGCCTTGTCAAG |
| | | GCAAGGCTGGCCAACCCATGGGTGGAGTTTAGCCAGGGACCGTTTCAG |
| | | ACAGATATTTGCATTGAGATAGTGTGGGGAAGGGG |
| PhTx ssODN1 | 907 | G*GGTGCTTCCTTTTATTCTTCATCCCTAGCCAGCCGCCGGCCCCTG |
| 5′ homology arm | | GCCTCACTGGATACTCTAAGACTATTGGTCAAGTTTGCCTT |
| PhTx ssODN1 | 908 | GTCAAGGCAAGGCTGGCCAACCCATGGGTGGAGTTTAGCCAGGGACCG |
| 3′ homology arm | | TTTCAGACAGATATTTGCATTGAGATAGTGTGGGGAAGGG*G |
| PhTx ssODN1 | 909 | G*GGTGCTTCCTTTTATTCTTCATCCCTAGCCAGCCGCCGGCCCCTG |
| | | GCCTCACTGGATACTCTAAGACTATTGGTCAAGTTTGCCTTGTCAAG |
| | | GCAAGGCTGGCCAACCCATGGGTGGAGTTTAGCCAGGGACCGTTTCAG |
| | | ACAGATATTTGCATTGAGATAGTGTGGGGAAGGG*G |

The homology arms flanking the deletion are indicated by bold [5′ homology arm] and underline [3′ homology arm]). Note the absence of the 13 bp sequence in ssODN1 and PhTx ssODN1.
*Represents modification by phosphorothioate.

ssODN1 and PhTx ssODN1 were co-delivered with RNP targeting HBG containing the Sp37 gRNA (HBG Sp37 RNP) or HBG Sp35 (HBG Sp35 RNP) to CB CD34+ cells. Co-delivery of the ssODN donor encoding the 13 nt deletion with HBG Sp35 RNP or HBG Sp37 RNP led to a 6-fold and 5-fold increase in gene editing of the target site, respectively, as determined by T7E1 analysis of the HBG2 PCR product (FIG. 4C). DNA sequencing analysis (Sanger sequencing) of the HBG2 PCR product indicated that 20% gene editing in cells that were treated with HBG Sp37 RNP and the PhTx modified ssODN1, with 15% deletions and 5% insertions (FIG. 4C, lower left panel). Further analysis of the specific type and size of deletions at the target site revealed that 75% of the total deletions detected contained the 13 nt deletion (which included deletion at c. −110 of the CAAT box in the proximal promoter), the absence of which is associated with elevation of HbF expression (FIG. 4C, lower right panel). The remaining ¼ of deletions were partial deletions that did not span the full 13 nt deletion. These data indicate that co-delivery of a homologous ssODN that is engineered to have a deletion supported precise gene editing (deletion) at HBG in human CD34⁺ cells.

### Example 3: Cas9 RNP Targeting the 13 nt Deletion Mutation Supports Gene Editing in Human Adult Mobilized Peripheral Blood Hematopoietic Stem/Progenitor Cells with Increased HBG Expression in Erythroblast Progeny

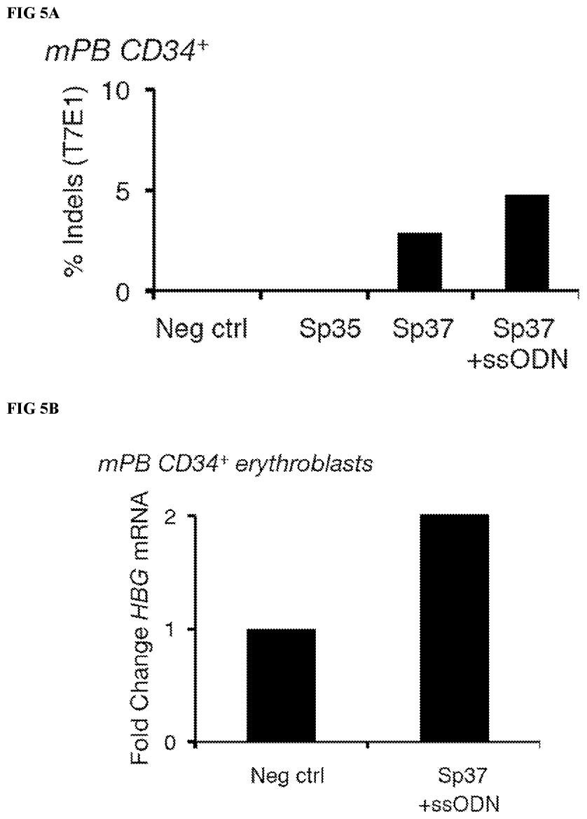

To determine whether editing HBG with Cas9 RNP complexed to Sp37 gRNA or Sp35 gRNA (i.e., the gRNAs that target the 13 nt deletion that is associated with HPFH) in the HBG promoter supports an increase in HBG expression in erythroid progeny of edited CD34⁺ cells, human adult CD34⁺ cells from mobilized peripheral blood (mPB) were electroporated with the RNPs. Briefly, mPB CD34⁺ cells were pre-stimulated for 2 days with human cytokines and PGE2 in StemSpan SFEM and then electroporated with Cas9 protein precomplexed to Sp35 and Sp37, respectively. T7E1 analysis of HBG PCR product indicated ˜3% indels detected for mPB CD34⁺ cells treated with RNP complexed to Sp37 while no editing was detected for cells that were treated with RNP complexed to Sp35 (FIG. 5A).

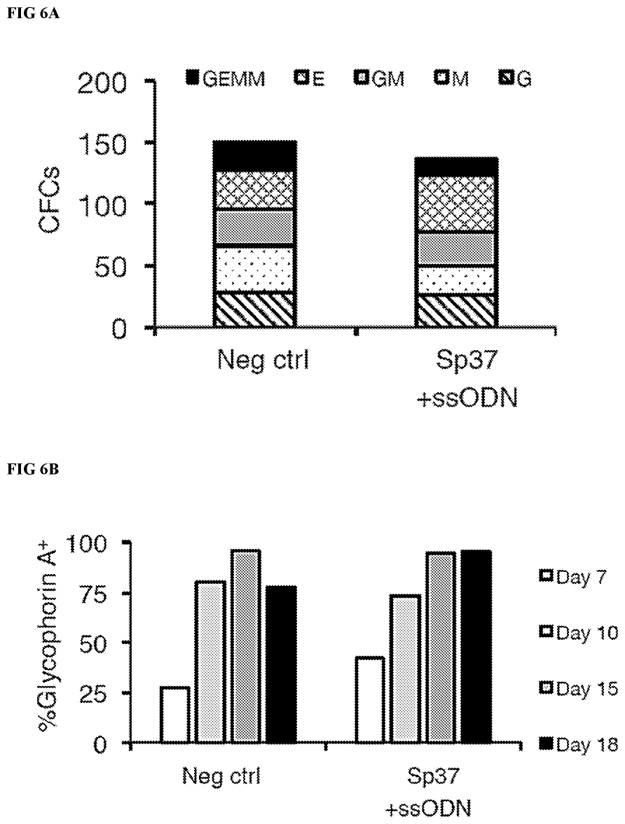

In order to increase gene editing at the target site and to increase the occurrence of the 13 nt deletion at the target site, PhTx ssODN1 (SEQ ID NO:909) was co-delivered with the precomplexed RNP targeting HBG containing the Sp37 gRNA. Co-delivery of the ssODN donor encoding the 13 nt deletion led to a nearly 2-fold increase in gene editing of the target site (FIG. 5A). To determine whether editing HBG increases production of fetal hemoglobin in erythroid progeny of edited adult CD34⁺ cells, the cells were differentiated into erythroblasts by culture for up to 18 days in the presence of human cytokines (erythropoietin, SCF, IL3), human plasma (Octoplas), and other supplements (hydrocortisone, heparin, transferrin). Over the time course of differentiation, mRNA was collected to evaluate HBG gene expression in the erythroid progeny of RNP treated mPB CD34⁺ cells and donor matched negative (untreated) controls. By day 7 of differentiation, erythroblast progeny of human CD34⁺ cells that were treated with HBG Sp37 RNP and 13 nt deletion encoding ssODN (˜5% indels detected in gDNA from the bulk cell population by T7E1 analysis) exhibited a 2-fold increase in HBG mRNA production (FIG. 5B). Importantly, CD34⁺ cells that were electroporated with HBG RNP maintained their ex vivo hematopoietic activity (i.e., no difference in the quantity or diversity of erythroid and myeloid colonies compared to untreated donor matched CD34⁺ cell negative control), as determined in hematopoietic colony forming cell (CFC) assays (FIG. 6A). Furthermore, the erythroblasts differentiated from RNP treated CD34⁺ cells maintained the kinetics of differentiation observed for donor matched untreated control cells as determined by flow analysis for acquisition of erythroid phenotype (% Glycophorin A+ cells) (FIG. 6B). These data indicate that targeted disruption of HBG1 IHBG2 proximal promoter region supported an increase in HBG expression in erythroid progeny of RNP treated adult hematopoietic stem/progenitor cells without altering differentiation potential.

### Example 4: Cas9 RNP Targeting the HPFH Mutation Supports Gene Editing in Human Adult Mobilized Peripheral Blood Hematopoietic Stem/Progenitor Cells with Increased HBG Expression in Erythroblast Progeny

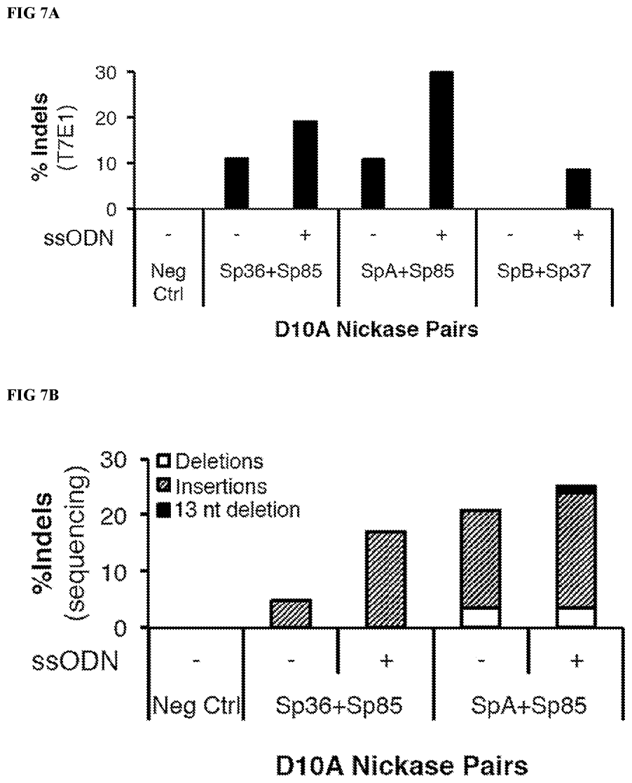

To determine whether co-delivery of paired nickase RNPs targeting HBG would increase targeted disruption of the proximal HBG promoter, mPB CD34+ cells were cultured for 2 days with human cytokines and PGE2 in StemSpan SFEM and then electroporated with S. pyogenes D10A Cas9 protein precomplexed to two gRNAs that target sites flanking the site of the 13 nt deletion. The targeting domain sequences for gRNAs used in nickase pairs in this example (including, without limitation, SpA, Sp85 and SpB) are presented in Table 7. D10A nickase pairs were selected such that the PAMs for the targets were oriented outward and the distance between the cut sites were <100 nt. gRNAs were complexed with D10A Cas9 protein to form RNP complexes and then human CD34+ cells and paired nickase were subject to electroporation. To determine whether co-delivery of an ssODN that encoded the 13 nt deletion would increase editing and introduction of the mutation into the cells, in some experiments, ssODN1 was added to the cell RNP mixture prior to electroporation. Approximately 3 days after electroporation, gDNA was extracted from the RNP treated cells and analyzed by T7E1 endonuclease assay and/or Sanger DNA sequencing of HBG2 PCR products amplified from the extracted gDNA. Of the three D10A nickase pairs tested, indels detected by T7E1 endonuclease analysis were increased for one nickase pair (gRNAs SpA+Sp85) samples for which ssODN1 was included (FIG. 7A). DNA sequencing analysis was performed on limited samples shown in FIG. 7A. DNA sequencing analysis showed up to ˜27% indels at the target site, with insertions as the dominant indel detected, followed by deletions of the targeted region (area between the cut sites of the paired nickases), and the 13 nt deletion mutation was also detected at a frequency of 2-3% when ssODN1 encoding the deletion was co-delivered (FIG. 7B). Silent, non-pathogenic SNPs were included in the ssODN1 donor template, and were detected in the sequences that contained the 13 nt deletion, indicating that creation of the HFPH mutation occurred through an HDR event.

### Example 5: D10A Paired RNPs Electroporated into Adult CD34+ Cells Supports Induction of HbF Protein in Erythroid Progeny

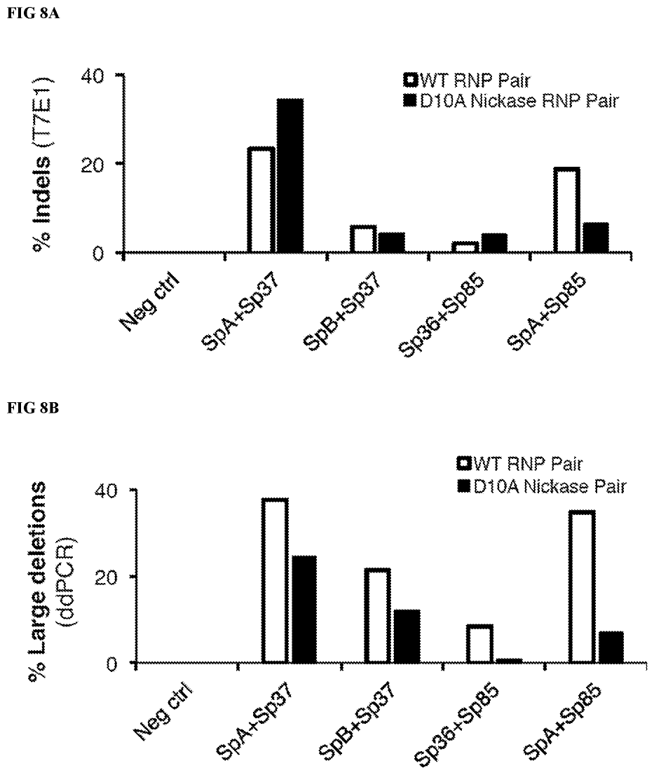

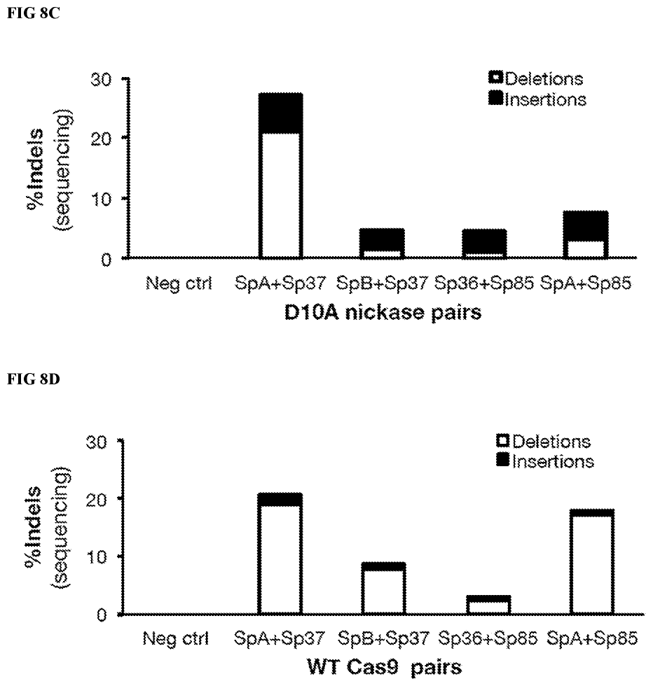

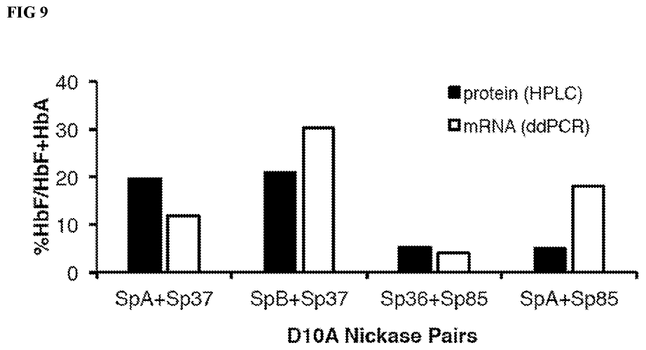

To further optimize editing conditions in mPB CD34⁺ cells at the target site and to evaluate editing in additional human cell donors, human mPB CD34⁺ cells were electroporated with D10A Cas9 and WT Cas9 paired RNPs targeting HBG. The most efficient guide pair for both D10A Cas9 and WT Cas9 RNPs was Sp37+SpA, which supported >30% indels as determined by T7E1 endonuclease analysis of HBG2 PCR products (FIG. 8A). Given that editing at both HBG1 and HBG2 could result in large deletions of HBG2 and the intergenic region between HBG2 and HBG1, indels were further characterized in order to capture local indels by T7E1 endonuclease assay and sequencing and large deletion by ddPCR analysis. Large deletions were detected in all samples at variable frequencies for both D10A Cas9 and WT Cas9 RNP nickase pairs (FIG. 8B). Illumina sequencing analysis of indels correlated with indels determined by T7E1 analysis (FIG. 8C-8D).

To determine whether CD34⁺ cells edited with dual nickases at the HBG promoter gave rise to erythroid progeny with elevated HbF expression, donor matched RNP treated and untreated controls were induced toward erythroid differentiation and then evaluated for maintenance of indels during differentiation and for expression of HbF mRNA and protein. The level of editing (as determined by T7E1 endonuclease assay) was evaluated over the first 2 weeks of erythroid differentiation in the progeny of RNP treated cells prior to enucleation. Indels were detected in the erythroid progeny at every time point assayed suggesting that the editing that occurred in the CD34⁺ cells was maintained during erythroid differentiation and that edited CD34⁺ cells maintain erythroid differentiation potential.

The levels of HBG mRNA (day 10 of differentiation) and HbF protein (day 20-23 of differentiation) were quantified by ddPCR and HPLC analysis (according to the HPLC method described in Chang 2017 at pp. 143-44, incorporated by reference herein), respectively (FIG. 9). A ˜2-fold increase (+40% in in HBG transcripts vs. unedited donor matched control) was observed for HBG: HBA ratio (data not shown) and the ratio of HbF/HbF+HbA (i.e. HBG mRNA/HGB+HBB mRNA) increased to 30% above the level detected in donor matched untreated control samples.

For the D10A Cas9 nickase pairs, upregulation of HbF mRNA and protein was detected in erythroid progeny (FIG. 9). With respect to HbF protein analysis, two pairs supported 20% HbF induction for two D10A nickase pairs. No HbF upregulation was detected in erythroid progeny of WT Cas9 RNP treated CD34⁺ cells (data not shown).

### Example 6: Increasing the Dose of RNP Increases Total Editing Efficiency in Human Adult CD34⁺ Cells at the HBG Locus

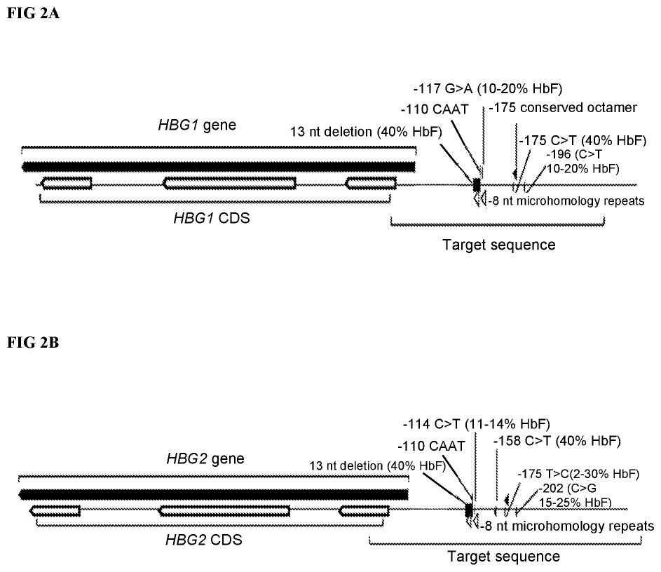

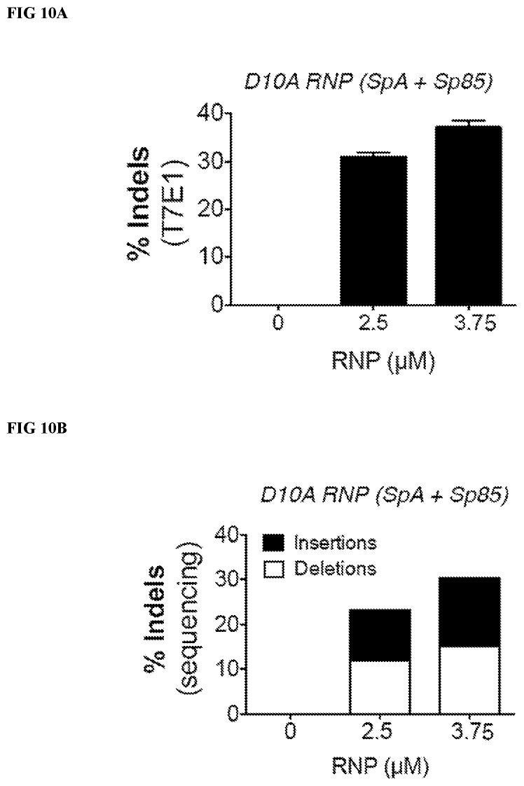

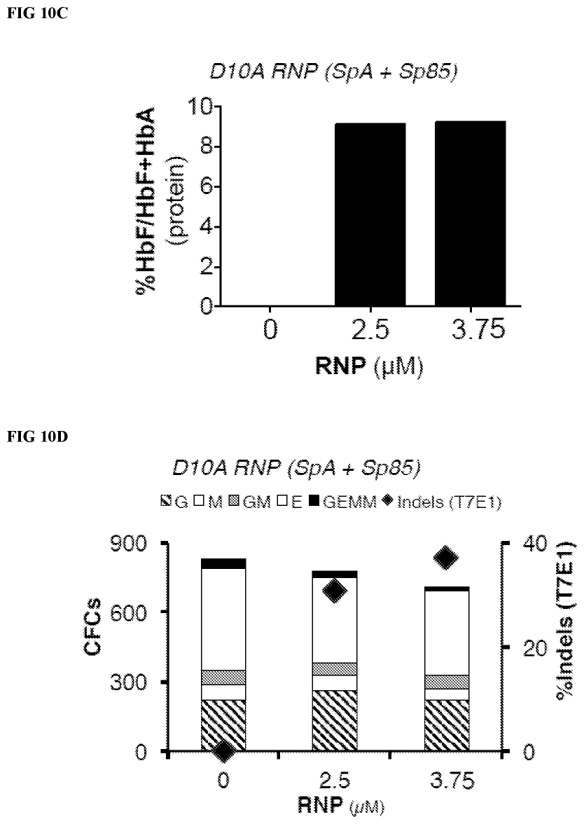

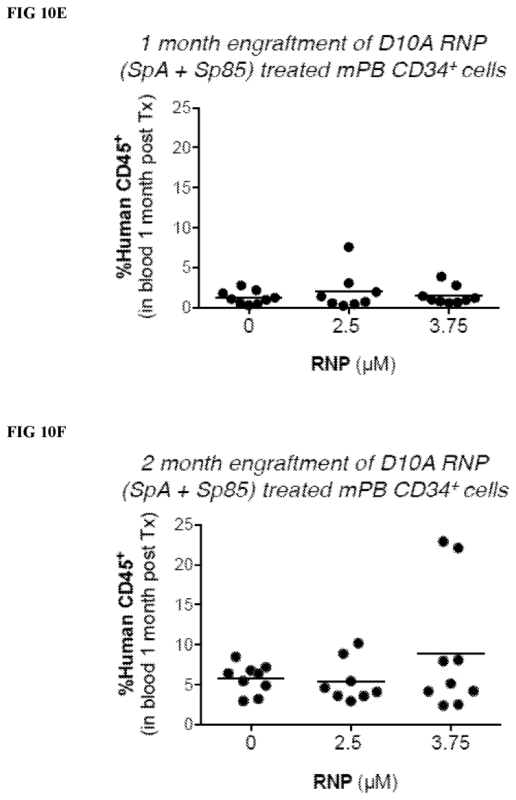

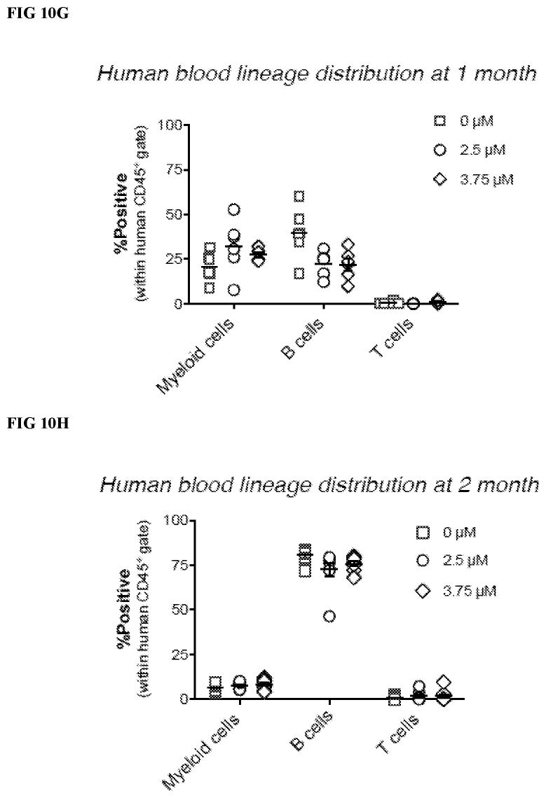

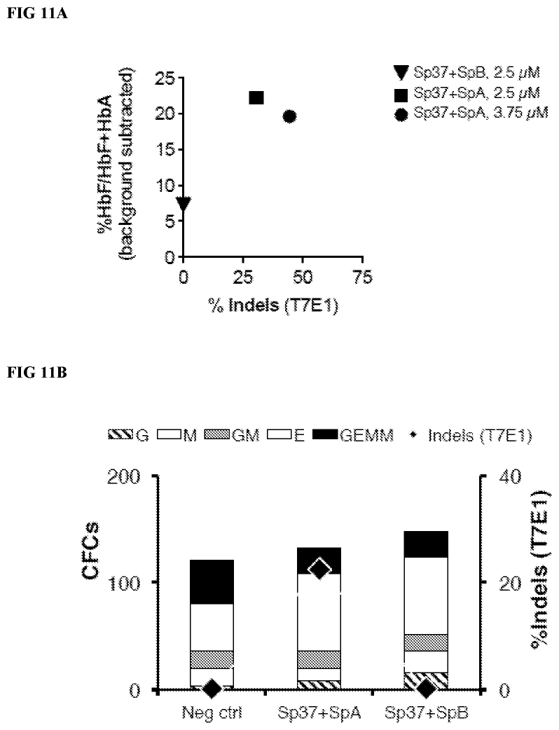

The concentration of D10A Cas9 RNP for the nickase pair SpA+Sp85 was increased (2.5 μM standard concentration and 3.7 μM) and delivered to mPB CD34⁺ cells by electroporation. The increased RNP concentration supported an increase in indels at the HBG target site to >30% (FIG. 10A) as determined by T7E1 endonuclease analysis of the HBG PCR product amplified for gDNA extracted 3 days after electroporation of CD34⁺ cells. Sequencing analysis indicated that increasing the RNP concentration increased insertions (FIG. 10B). Erythroid progeny of RNP treated CD34+ cells also had an increase in HbF protein production (FIG. 10C). Importantly, the hematopoietic colony forming potential was maintained after editing (FIG. 10D). These cells were then transplanted into immunodeficient mice and their engraftment 1 month (FIG. 10E) and 2 months (FIG. 10F) after transplantation was evaluated by sampling the peripheral blood and measuring the percentage of human CD45⁺ cells. Early engraftment data showed no difference in engraftment between recipient cohorts of donor matched untreated controls (0 μM RNP) and mice transplanted with RNP treated cells. Furthermore, there was no difference in human blood lineage distribution (myeloid, B cell, T cell) within the human CD45⁺ fraction among cohorts at indicated time points (FIG. 10G-H).

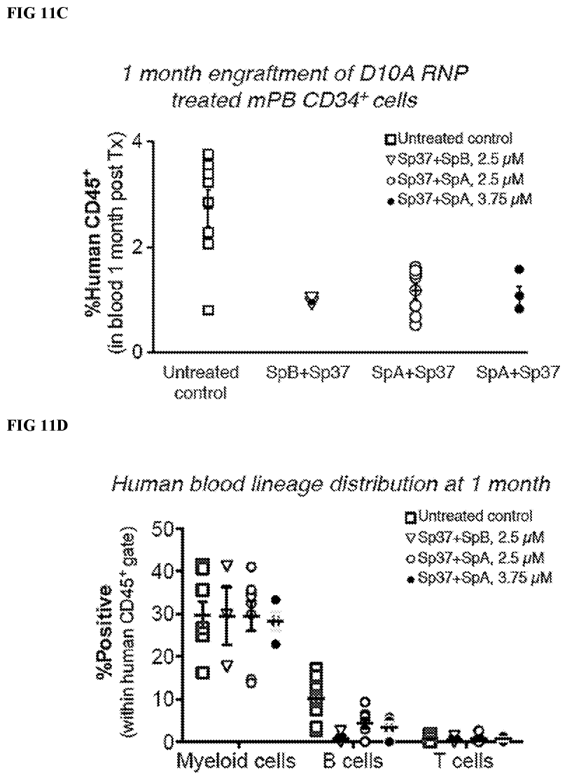

Two additional D10A nickase pairs were also tested in RNP dose response studies in adult mPB CD34⁺ cells (Sp37+SpA, Sp37+SpB). Here, mPB CD34+ cells were electroporated with D10A paired nickases delivered at 0, 2.5, and 3.75 μM of total RNP. RNP treated cells were differentiated into erythroid progeny and the HbF protein levels (% HbF/HbF+HbA) were analyzed by HPLC analysis. The indel frequency detected in CD34⁺ cells was plotted with the HbF levels detected in erythroid progeny in order to correlate editing and HbF induction (FIG. 11A). RNP treated and untreated control mPB CD34⁺ cells were also differentiated into colonies to evaluate ex vivo hematopoietic activity. Colony forming cell (CFC) activity was maintained for the progeny of RNP treated and donor matched untreated control CD34⁺ cells (FIG. 11B). There was no difference in the percentage of human CD45⁺ cells in the mouse peripheral blood 1 month after transplantation and no difference in blood lineage distribution (FIG. 11C-D) for cells exposed to different D10A RNP pairs at different doses compared to untreated donor matched control CD34⁺ cells.

### Example 7: Co-Delivery of RNP Targeting the Erythroid Specific Enhancer of BCL11A and a Non-Specific (N) Single Strand Deoxynucleotide Sequence or Paired RNPs Increases Gene Editing in Human CD34⁺ Cells and Supports Induction of Fetal Hemoglobin Expression in Erythroid Progeny

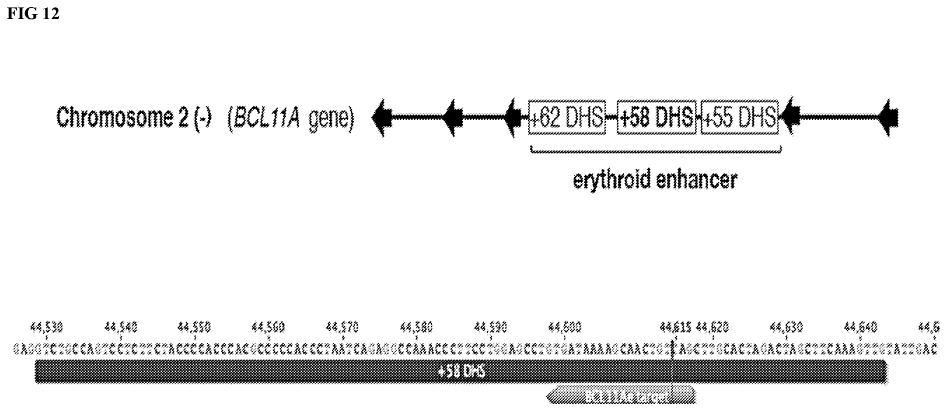

Fetal hemoglobin expression can be induced through targeted disruption of the erythroid cell specific expression of a transcriptional repressor, BCL11A (Canvers 2015). One potential strategy to increase HbF expression through a gene editing strategy is to multiplex gene editing for introduction of 13 nt deletion associated in the HBG proximal promoter and also for targeted disruption of the GATA1 binding motif in the erythroid specific enhancer of BCL11A that is in the +58 DHS region of intron 2 of the BCL11A gene (FIG. 12). In order to accomplish this multiplex strategy to increase HbF expression through multiplex gene editing, the effect of disruption of BCL11A erythroid enhancer (BCL11Ae) must first be determined as a single editing event.

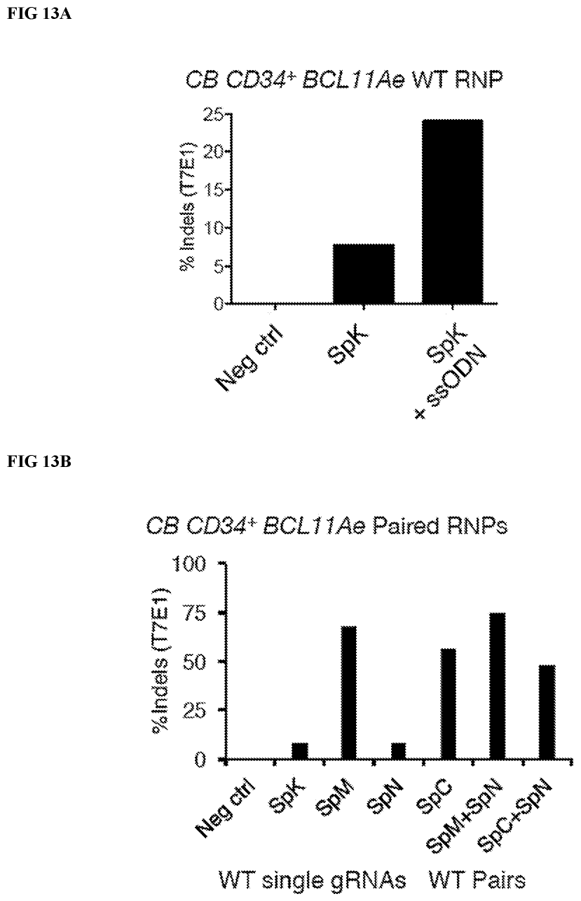

In this experiment, CB CD34⁺ cells were electroporated with S. pyogenes WT Cas9 complexed to in vitro transcribed sgRNA targeting the GATA1 motif in the +58 DHS region of intron 2 of BCL11A gene (gRNA SpK, Table 9) (FIG. 13A). To determine whether co-delivery of a non-target specific ssODN would increase editing of the target sequence, BCL11Ae RNP was co-delivered with ssODN (which is nonhomologous to the BCL11Ae target sequence) in CB CD34⁺ cells. T7E1 analysis of BCL11A erythroid enhancer PCR product from gDNA extracted from CB CD34⁺ cells treated with BCL11Ae RNP indicated that ˜5% indels was achieved (FIG. 13A). Co-delivery of BCL11Ae RNP with a non-target specific ssODN increase in indels by 5-fold to 20% as detected by T7E1 endonuclease analysis. Illumina sequencing analysis indicated that >90% of edits had disruption of the GATA1 motif in the +DHS 58 region enhancer in intron 2 of the BCL11A gene (data not shown). To increase editing, human CB CD34⁺ cells were electroporated with WT Cas9 RNP (single gRNAs complexed to WT Cas9) or with WT Cas9 paired RNPs (paired gRNAs complexed to WT Cas9), so that the cut sites in each pair flank the target site for excision of the GATA1 motif (gRNAs SpC, SpK, SpM, SpN) (Table 9). Two of the single gRNAs and two pairs had >50% indels as determined by T7E1 endonuclease analysis (FIG. 13B).

TABLE 9
Select gRNA sequences targeting BCL11A erythroid enhancer for screening in
CD34⁺ cells

| | | | | Targeting domain | |
|---|---|---|---|---|---|
| | Targeting | | Targeting domain | sequence plus | |
| gRNA | domain sequence | Targeting domain | sequence plus PAM | PAM (NGG) | |
| ID | (RNA) | sequence (DNA) | (NGG)(RNA) | (DNA) | Sense |
| SpK | CUAACAGUUGC | CTAACAGTTGC | CUAACAGUUGCU | CTAACAGTTGCTT | Antisense |
| | UUUUAUCAC | TTTTATCAC | UUUAUCACAGG | TTATCACAGG | |
| | (SEQ ID NO: 952) | (SEQ ID NO: 956) | (SEQ ID NO: 960) | (SEQ ID NO: 964) | |
| SpM | GGGCGUGGGUG | GGGCGTGGGT | GGGCGUGGGUGG | GGGCGTGGGTGG | Antisense |
| | GGGUAGAAG | GGGGTAGAAG | GGUAGAAGAGG | GGTAGAAGAGG | |
| | (SEQ ID NO: 953) | (SEQ ID NO: 957) | (SEQ ID NO: 961) | (SEQ ID NO: 965) | |
| SpN | CUCUUAGACAU | CTCTTAGACAT | CUCUUAGACAUA | CTCTTAGACATAA | Antisense |
| | AACACACCA | AACACACCA | ACACACCAGGG | CACACCAGGG | |
| | (SEQ ID NO: 954) | (SEQ ID NO: 958) | (SEQ ID NO: 962) | (SEQ ID NO: 966) | |
| SpC | AUCAGAGGCCA | ATCAGAGGCC | AUCAGAGGCCAA | ATCAGAGGCCAA | Sense |
| | AACCCUUCC | AAACCCTTCC | ACCCUUCCUGG | ACCCTTCCTGG | |
| | (SEQ ID NO: 955) | (SEQ ID NO: 959) | (SEQ ID NO: 963) | (SEQ ID NO: 967) | |

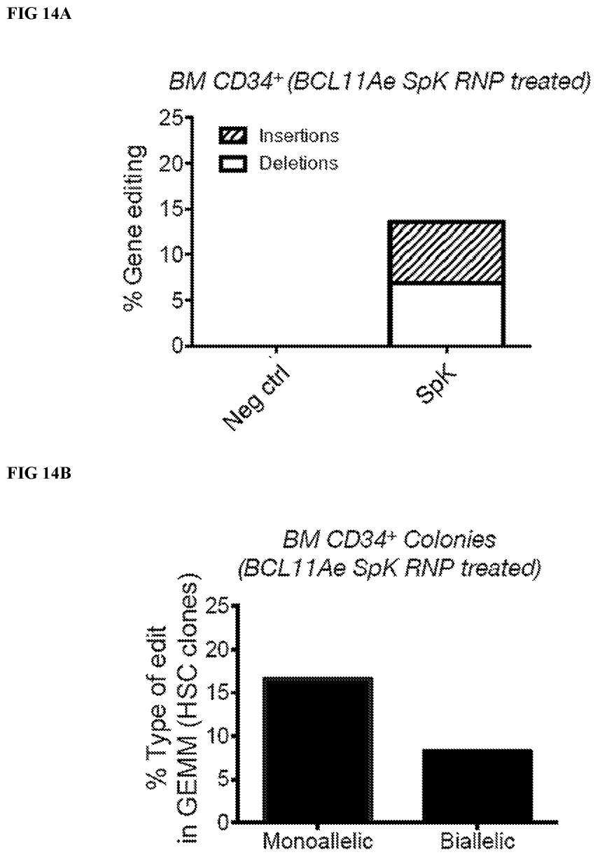

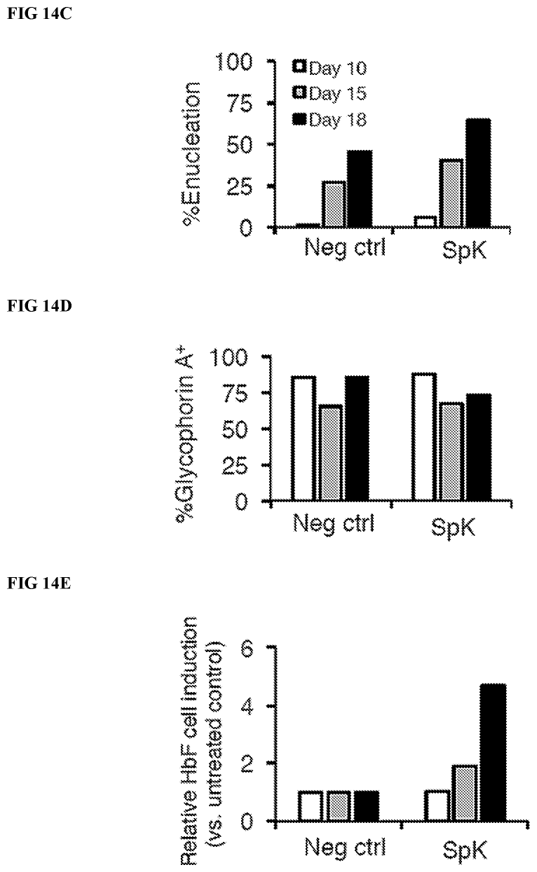

Next, human adult bone marrow CD34⁺ cells were electroporated with the BCL11Ae RNP. DNA sequencing analysis of the BCL11A PCR product amplified from gDNA extracted from marrow CD34⁺ cells indicated 15% gene editing comprised of insertions and deletions (FIG. 14A). Importantly, all deletions resulted in deletion of the GATA1 motif and all insertions disrupted GATA1 motif through addition of a small number of bp in the motif. CD34⁺ cells were plated into colony forming assays and the mixed hematopoietic colonies (GEMMs), which correspond to CD34⁺ cell clones, were picked. gDNA was isolated and analyzed by Illumina sequencing to quantify monoallelic and biallelic disruption of the target site. Most GEMMs differentiated from the CD34⁺ cell clones had monoallelic disruption and biallelic disruption was also detected, with the overall indel rate˜2/3 higher compared to what was detected in the bulk CD34+ cell population (FIG. 14B). This was likely a reflection of the percentage of common myeloid progenitors (CMPs) that give rise to GEMMs that make up a larger fraction of the heterogenous CD34⁺ cells versus the other lineages present, but not captured/differentiated in the short-term CFC assays. The RNP treated marrow CD34⁺ cells also maintained similar kinetics of erythroid maturation (enucleation, FIG. 14C) and differentiation (phenotype acquisition, FIG. 14D) compared to donor matched untreated control cells. Erythroid progeny of edited marrow CD34⁺ cells exhibited ˜5-fold increase in HbF induction as determined by flow cytometry analysis (FIG. 14E).

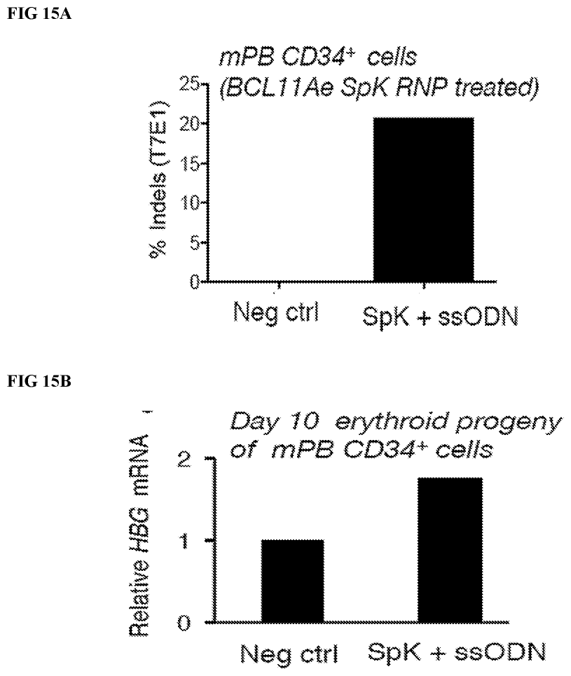

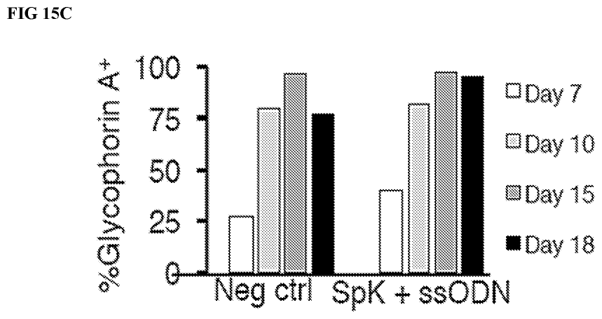

Gene editing and induction of fetal hemoglobin was also evaluated in human adult mPB CD34+ cells. Co-delivery of BCL11Ae RNP and nonspecific ssODN supported ˜20% indels at the target site (FIG. 15A). To evaluate early induction of fetal hemoglobin in erythroid progeny of edited cells, mPB CD34+ cells were differentiated into erythroblasts and induction of fetal hemoglobin transcription (HBG mRNA) was evaluated by qRT-PCR analysis. The erythroid progeny of BCL11Ae RNP treated CD34⁺ cells exhibited a 2-fold induction of HBG mRNA compared to untreated controls, suggesting induction of fetal hemoglobin expression (FIG. 15B). The RNP treated marrow CD34⁺ cells also maintained similar kinetics of differentiation (phenotype acquisition, FIG. 15C) compared to donor matched untreated control cells.

### Example 8: RNP Targeting the CAAT Box have Higher Activity when Complexed with Chemically Synthesized Guide RNA than In Vitro Transcribed Synthesized gRNA

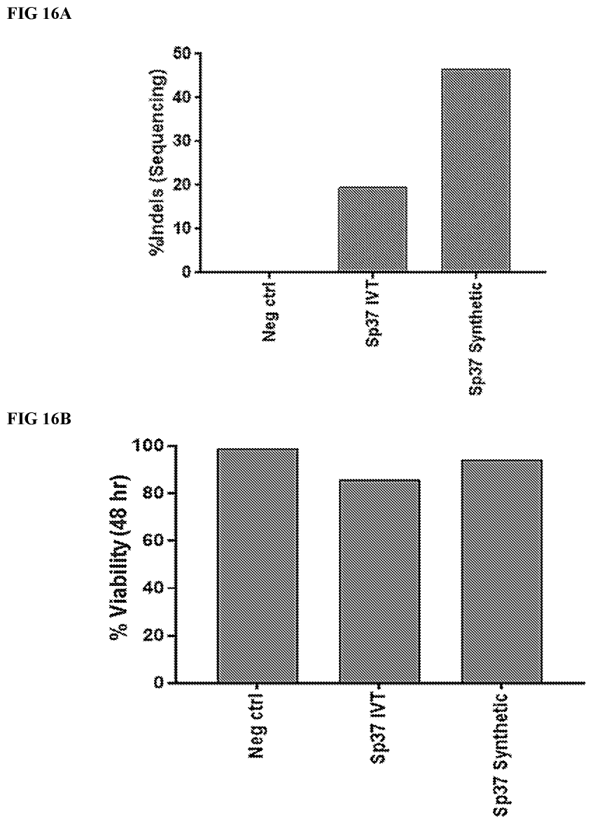

To determine whether complexing Cas9 RNP with a chemically synthesized gRNA enhances editing in the promoter region of HBG, Sp37 gRNA (i.e., a gRNA that targets the 13 nt deletion associated with HPFH; targeting domain set forth in SEQ ID NO:933) was in vitro transcribed (IVT) or chemically synthesized. Human adult CD34+ cells from mobilized peripheral blood (mPB) were electroporated with the RNPs. Briefly, mPB CD34+ cells were prestimulated for 3 days with human cytokines and PGE2 in StemSpan SFEM and then electroporated with Cas9 protein precomplexed to Sp37 (IVT) or Sp37 (synthetic). The use of synthetic guide RNA supported an increase in indels at the HBG target of more than 2 fold (FIG. 16A) as determined by sequencing analysis of the HBG PCR product amplified from gDNA extracted 4 days after electroporation of CD34+ cells. The viability of the cells at 48 hours post electroporation was not affected by the use of a chemically synthesized gRNA (FIG. 16A).

### Example 9: Sequence Mismatches and End Modifications of Synthetic Guide RNAs Increase the Activity of RNP Targeting the CAAT Box

To evaluate if end modifications of chemically synthesized gRNA enhance the editing of RNP in the promoter region of HBG, the Sp35 gRNA (i.e., a gRNA that targets the 13 nt deletion associated with HPFH; full length sequence set forth in SEQ ID NO:970, targeting domain sequence set forth in SEQ ID NO:339) was chemically synthesized. One or a combination of the following modifications were made at one or both of the ends of the Sp35 gRNA sequence: 3 nucleotides with a phosphorothioate (PhTx) group and 3 nucleotides with a 2′-O-methyl group (Table 10).

To evaluate if truncations of the protospacer or nucleotide mismatches enhance the editing of RNP in the HBG promoter, 5′ end truncations of the Sp35 gRNA were chemically synthesized (Sp35 97mer (SEQ ID NO:976), Sp35 98mer (SEQ ID NO:977), Sp35 99mer (SEQ ID NO:978), Table 10). An Sp35 guide RNA with a C-to-A sequence substitution at the 5′ end was also synthesized (Sp35-5′ C->A (SEQ ID NO:979), Table 10).

TABLE 10
Sequences of chemically synthesized gRNAs targeting the CAAT box, with
end modification, sequence substitution or truncations

| Name | Oli-ID | gRNA sequence (RNA) | gRNA sequence (DNA) |
|---|---|---|---|
| Sp35 unmodified | OLI7066 | CUUGUCAAGGCUAUUG | CTTGTCAAGGCTATTG |
| | | GUCAGUUUUAGAGCUA | GTCAGTTTTAGAGCTA |
| | | GAAAUAGCAAGUUAAA | GAAATAGCAAGTTAAA |
| | | AUAAGGCUAGUCCGUU | ATAAGGCTAGTCCGTT |
| | | AUCAACUUGAAAAAGU | ATCAACTTGAAAAAGT |
| | | GGCACCGAGUCGGUGCU | GGCACCGAGTCGGTGC |
| | | UUU (SEQ ID NO: 970) | TTTT (SEQ ID NO: 980) |
| Sp35 5′ PS | OLI8568 | C*U*U*GUCAAGGCUAU | C*T*T*GTCAAGGCTAT |
| | | UGGUCAGUUUUAGAGC | TGGTCAGTTTTAGAGC |
| | | UAGAAAUAGCAAGUUA | TAGAAATAGCAAGTTA |
| | | AAAUAAGGCUAGUCCG | AAATAAGGCTAGTCCG |
| | | UUAUCAACUUGAAAAA | TTATCAACTTGAAAAA |
| | | GUGGCACCGAGUCGGUG | GTGGCACCGAGTCGGT |
| | | CUUUU (SEQ ID NO: 971) | GCTTTT (SEQ ID |
| | | | NO: 981) |
| Sp35 5′ OMe | OLI8569 | mCmUmUGUCAAGGCUA | mCmTmTGTCAAGGCTA |
| | | UUGGUCAGUUUUAGAG | TTGGTCAGTTTTAGAG |
| | | CUAGAAAUAGCAAGUU | CTAGAAATAGCAAGTT |
| | | AAAAUAAGGCUAGUCC | AAAATAAGGCTAGTCC |
| | | GUUAUCAACUUGAAAA | GTTATCAACTTGAAAA |
| | | AGUGGCACCGAGUCGGU | AGTGGCACCGAGTCGG |
| | | GCUUUU (SEQ ID NO: 972) | TGCTTTT (SEQ ID |
| | | | NO: 982) |
| Sp35 3′ P SOMe | OLI8396 | CUUGUCAAGGCUAUUG | CTTGTCAAGGCTATTG |
| | | GUCAGUUUUAGAGCUA | GTCAGTTTTAGAGCTA |
| | | GAAAUAGCAAGUUAAA | GAAATAGCAAGTTAAA |
| | | AUAAGGCUAGUCCGUU | ATAAGGCTAGTCCGTT |
| | | AUCAACUUGAAAAAGU | ATCAACTTGAAAAAGT |
| | | GGCACCGAGUCGGUGCm | GGCACCGAGTCGGTGC |
| | | U*mU*mU*U (SEQ ID | mT*mT*mT*T (SEQ ID |
| | | NO: 973) | NO: 983) |
| Sp35 5′ PSOMe | OLI8395 | mC*mU*mU*GUCAAGGC | mC*mT*mT*GTCAAGGC |
| | | UAUUGGUCAGUUUUAG | TATTGGTCAGTTTTAGA |
| | | AGCUAGAAAUAGCAAG | GCTAGAAATAGCAAGT |
| | | UUAAAAUAAGGCUAGU | TAAAATAAGGCTAGTC |
| | | CCGUUAUCAACUUGAAA | CGTTATCAACTTGAAA |
| | | AAGUGGCACCGAGUCGG | AAGTGGCACCGAGTCG |
| | | UGCUUUU (SEQ ID | GTGCTTTT (SEQ ID |
| | | NO: 974) | NO: 984) |
| Sp35 5′-3′ PSOMe | OLI8394 | mC*mU*mU*GUCAAGGC | mC*mT*mT*GTCAAGGC |
| | | UAUUGGUCAGUUUUAG | TATTGGTCAGTTTTAGA |
| | | AGCUAGAAAUAGCAAG | GCTAGAAATAGCAAGT |
| | | UUAAAAUAAGGCUAGU | TAAAATAAGGCTAGTC |
| | | CCGUUAUCAACUUGAAA | CGTTATCAACTTGAAA |
| | | AAGUGGCACCGAGUCGG | AAGTGGCACCGAGTCG |
| | | UGCmU*mU*mU*U (SEQ | GTGCmT*mT*mT*T |
| | | ID NO: 975) | (SEQ ID NO: 985) |
| Sp35 97mer | OLI8018 | GUCAAGGCUAUUGGUC | GTCAAGGCTATTGGTC |
| | | AGUUUUAGAGCUAGAA | AGTTTTAGAGCTAGAA |
| | | AUAGCAAGUUAAAAUA | ATAGCAAGTTAAAATA |
| | | AGGCUAGUCCGUUAUCA | AGGCTAGTCCGTTATC |
| | | ACUUGAAAAAGUGGCA | AACTTGAAAAAGTGGC |
| | | CCGAGUCGGUGCUUUU | ACCGAGTCGGTGCTTT |
| | | (SEQ ID NO: 976) | T (SEQ ID NO: 986) |
| Sp35 98mer | OLI8019 | UGUCAAGGCUAUUGGU | TGTCAAGGCTATTGGT |
| | | CAGUUUUAGAGCUAGA | CAGTTTTAGAGCTAGA |
| | | AAUAGCAAGUUAAAAU | AATAGCAAGTTAAAAT |
| | | AAGGCUAGUCCGUUAUC | AAGGCTAGTCCGTTAT |
| | | AACUUGAAAAAGUGGC | CAACTTGAAAAAGTGG |
| | | ACCGAGUCGGUGCUUUU | CACCGAGTCGGTGCTT |
| | | (SEQ ID NO: 977) | TT (SEQ ID NO: 987) |
| Sp35 99mer | OLI8020 | UUGUCAAGGCUAUUGG | TTGTCAAGGCTATTGG |
| | | UCAGUUUUAGAGCUAG | TCAGTTTTAGAGCTAG |
| | | AAAUAGCAAGUUAAAA | AAATAGCAAGTTAAAA |
| | | UAAGGCUAGUCCGUUA | TAAGGCTAGTCCGTTA |
| | | UCAACUUGAAAAAGUG | TCAACTTGAAAAAGTG |
| | | GCACCGAGUCGGUGCUU | GCACCGAGTCGGTGCT |
| | | UU (SEQ ID NO: 978) | TTT (SEQ ID NO: 988) |
| Sp35-5′ C→A | OLI8397 | AUUGUCAAGGCUAUUG | ATTGTCAAGGCTATTG |
| | | GUCAGUUUUAGAGCUA | GTCAGTTTTAGAGCTA |
| | | GAAAUAGCAAGUUAAA | GAAATAGCAAGTTAAA |
| | | AUAAGGCUAGUCCGUU | ATAAGGCTAGTCCGTT |
| | | AUCAACUUGAAAAAGU | ATCAACTTGAAAAAGT |
| | | GGCACCGAGUCGGUGCU | GGCACCGAGTCGGTGC |
| | | UUU (SEQ ID NO: 979) | TTTT (SEQ ID NO: 989) |

*: Represents phosphorothioate modification.
m: Represents, 2-o-methyl modification.

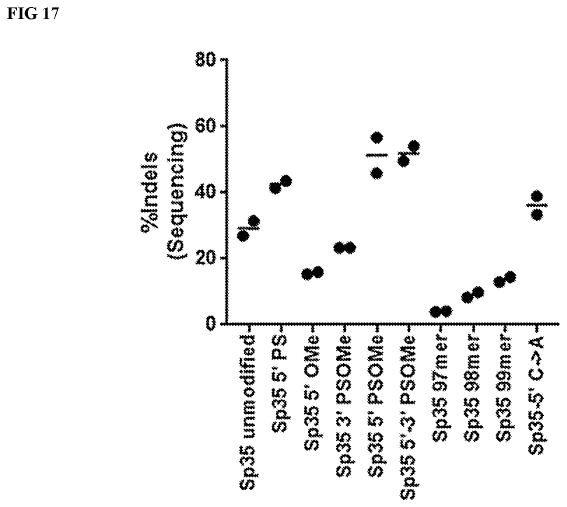

Next, Cas9 RNP were complexed with Sp35 gRNA or modified Sp35 guide RNAs (Table 10) and 7.27 μM RNP were electroporated to human adult CD34+ cells from mobilized peripheral blood (mPB). Sequence analysis of the HBG PCR product amplified from gDNA extracted post-electroporation was performed. Increased editing was observed with Sp35 guide RNAs containing 5′ and 5′+3′ modifications, as well as with the C-to-A substitution when compared to the unmodified Sp35 guide RNA (FIG. 17).

### Example 10: Co-Delivery of Cas9 RNP Containing Chemically Synthesized Guide RNA Targeting the 13 nt Deletion Mutation with ssODN Donors Supports Precise Gene Editing in Human Hematopoietic Stem/Progenitor Cells and Increased Gamma-Globin Expression in the Erythroid Progeny

To increase gene editing and the occurrence of the 13 nt deletion at the target site, single strand deoxynucleotide donor repair templates (ssODN) that encoded 90 nucleotides of homology on each side of the targeted deletion site were generated. The ssODNs were modified to contain phosphorothioates (PhTx) at the 5′ and 3′ ends (i.e., 01116414 (SEQ ID NO:990), 01116412 (SEQ ID NO:993), respectively, Table 11). The ssODNs were designed to “encode” the 13 nt deletion with sequence homology arms flanking this absent sequence to create a perfect deletion.

TABLE 11
Single strand deoxynucleotide donor repair templates used in combination
with OLI7066-sp35-RNP

| | | SEQ ID | |
|---|---|---|---|
| Name | Oli-ID | NO | Sequence |
| PhTx ssODN | OLI16414 | 990 | A*AGGGTGCTTCCTTTTATTCTTCATCCCT |
| 13 nt-Positive | | | AGCCAGCCGCCGGCCCCTGGCCTCACTG |
| strand | | | GATACTCTAAGACTATTGGTCAAGTTTGC |
| | | | CTTGTCAAGGCAAGGCTGGCCAACCCATGG |
| | | | GTGGAGTTTAGCCAGGGACCGTTTCAGACA |
| | | | GATATTTGCATTGAGATAGTGTGGGGAAGG |
| | | | GGC*C |
| 5′ homology | 5′ homology | 991 | A*AGGGTGCTTCCTTTTATTCTTCATCCCT |
| arm: PhTx | arm OLI16414 | | AGCCAGCCGCCGGCCCCTGGCCTCACTG |
| ssODN 13 nt- | | | GATACTCTAAGACTATTGGTCAAGTTTGC |
| Positive strand | | | CTTG |
| 3′ homology | 3′ homology | 992 | TCAAGGCAAGGCTGGCCAACCCATGGGTGG |
| arm: PhTx | arm OLI16414 | | AGTTTAGCCAGGGACCGTTTCAGACAGATA |
| ssODN 13 nt- | | | TTTGCATTGAGATAGTGTGGGGAAGGGGC*C |
| Positive strand | | | |
| PhTx ssODN | OLI16412 | 993 | G*GCCCCTTCCCCACACTATCTCAATGCA |
| 13 nt- | | | AATATCTGTCTGAAACGGTCCCTGGCTAA |
| Negative strand | | | ACTCCACCCATGGGTTGGCCAGCCTTGCC |
| | | | TTGACAAGGCAAACTTGACCAATAGTCTTA |
| | | | GAGTATCCAGTGAGGCCAGGGGCCGGCGGC |
| | | | TGGCTAGGGATGAAGAATAAAAGGAAGCAC |
| | | | CCT*T |
| 5′ homology | 5′ homology | 994 | G*GCCCCTTCCCCACACTATCTCAATGCA |
| arm: PhTx | arm OLI16412 | | AATATCTGTCTGAAACGGTCCCTGGCTAA |
| ssODN 13 nt- | | | ACTCCACCCATGGGTTGGCCAGCCTTGCC |
| Negative strand | | | TTGA |
| 3′ homology | 3′ homology | 995 | CAAGGCAAACTTGACCAATAGTCTTAGAGT |
| arm: PhTx | arm OLI16412 | | ATCCAGTGAGGCCAGGGGCCGGCGGCTGGC |
| ssODN 13 nt- | | | TAGGGATGAAGAATAAAAGGAAGCACCCT* |
| Negative strand | | | T |

The homology arms flanking the deletion are indicated by bold [5′ homology arm] and underline [3′ homology arm]). Note the absence of the 13 bp sequence in OLI16414 and OLI16412.
*: Represents phosphorothioate modification.

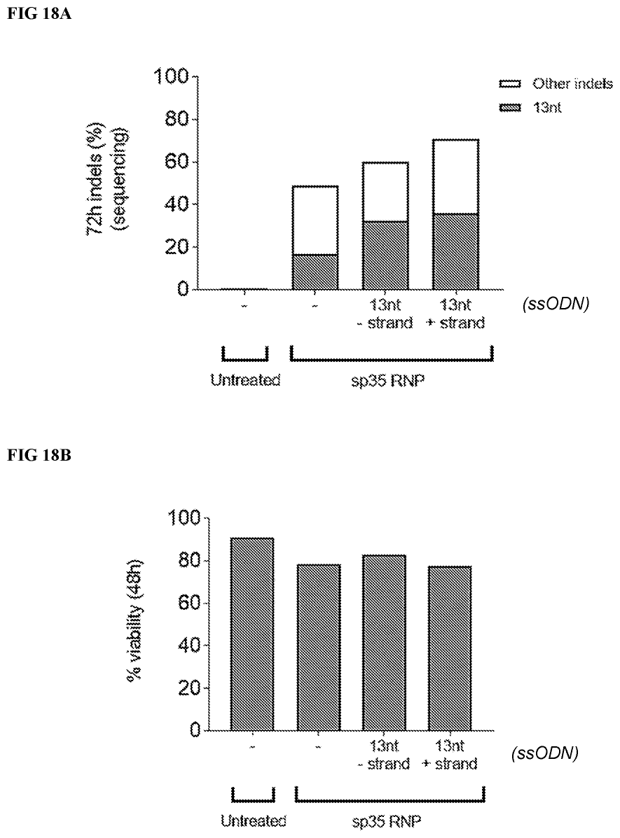

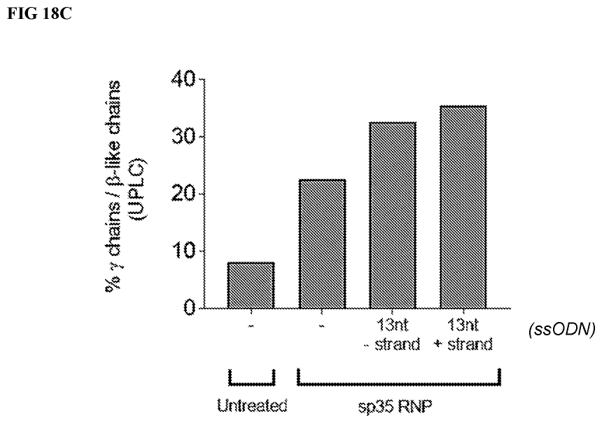

01116414 (SEQ ID NO:990) and 01116412 (SEQ ID NO:993) were co-delivered with RNP targeting HBG containing the chemically synthesized Sp35 gRNA (SEQ ID NO:970) (“OLI7066-sp35-RNP”) to human adult CD34+ cells from mobilized peripheral blood (mPB). Briefly, mPB CD34+ cells were prestimulated for 2 days with human cytokines in X-Vivo-10 and then electroporated with a mixture composed of an ssODN donor and Cas9 protein precomplexed to Sp35 (OLI7066-sp35-RNP). Co-delivery of 2.5 μM of the ssODN donor encoding the 13 nt deletion as a positive strand donor (OLI16414 (SEQ ID NO:990)) or negative strand donor (OLI16412 (SEQ ID NO:993)) with 2 μM of OLI7066-sp35-RNP enhanced the editing frequency from 48.7% (without ssODN donor) to 60.11% and 70.7% respectively, as determined by sequencing analysis of the HBG PCR product from genomic DNA extracted at 72 hours post-electroporation (FIG. 18A).

Further analysis of the specific type and size of deletions at the target site revealed that 32.6% and 36.0% of the alleles carried the precise 13 nt deletion when OLI7066-sp35-RNP was co-delivered with the 13 nt positive strand donor (01116414 (SEQ ID NO:990)) and the 13 nt negative strand donor (01116412 (SEQ ID NO:993)), respectively, instead of 16.9% when OLI7066-sp35-RNP was delivered alone (FIG. 18A). No reduction in viability of CD34 cells at 48 hours post electroporation was observed when OLI7066-sp35-RNP was co-delivered with the ssODNs (FIG. 18B).

To determine whether co-delivering OLI7066-sp35-RNP with ssODNs “encoding” the 13 nt deletion increased the production of fetal hemoglobin in the erythroid progeny of edited adult CD34⁺ cells, the cells were differentiated into erythroblasts by culture for 18 days in the presence of human cytokines (erythropoietin, SCF, IL3), human plasma (Octoplas), and other supplements (hydrocortisone, heparin, transferrin, insulin). At day 18, the relative expression levels of gamma-globin chains over total beta-like globin chains (gamma chains/[gamma chains+beta chain]) was increased from 8.1% in the untreated sample to 22.6%, 32.6% and 35.4% when the CD34 cells were initially electroporated with OLI7066-sp35-RNP alone, OLI7066-sp35-RNP in combination with the negative strand ssODN donor (OLI16412 (SEQ ID NO:993)), and OLI7066-sp35-RNP in combination with the positive strand ssODN donor (01116414 (SEQ ID NO:990)), respectively, as determined by ultra-performance liquid chromatography (UPLC) analysis of the cell lysates (FIG. 18C). These results demonstrate that co-delivery of a chemically synthesized guide RNA targeting the HBG distal CAAT-box with a homologous ssODN that is engineered to have a deletion supported precise gene editing (deletion) at HBG in human mPB+CD34 cells, resulting in increased level of HBG expression.

### Example 11: Infusion of Edited mPB CD34+ Cells into NOD,B6.SCID Il2rγ−/− Kit(W41/W41) Mice Results in Long Term Engraftment and HbF Induction

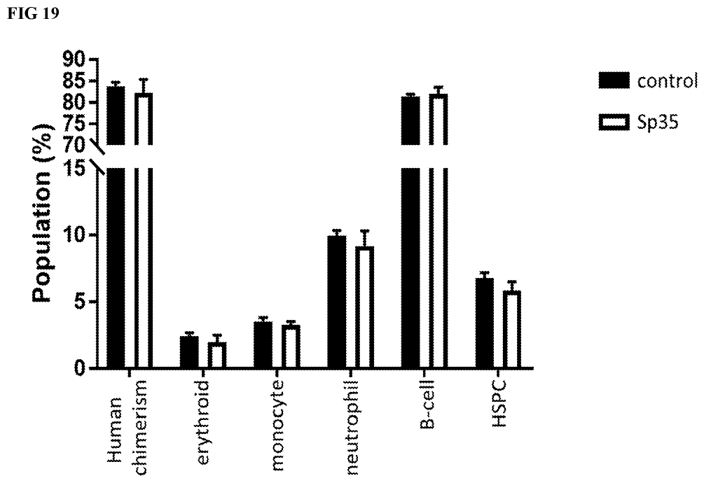

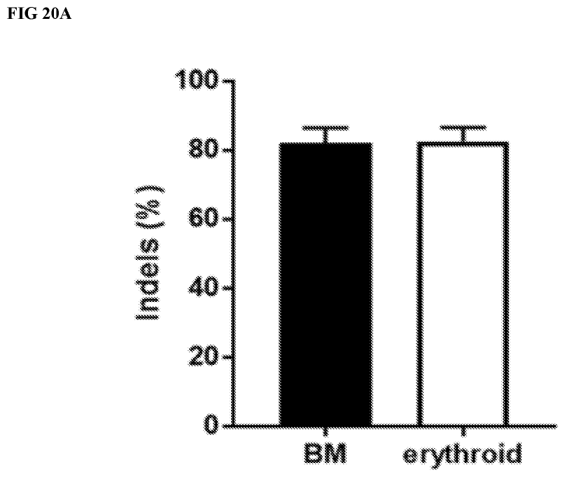

To determine whether delivery of Sp35 RNP (comprising Sp35 gRNA (SEQ ID NO:339, Table 7) edits the distal CCAAT-box target site in long term repopulating hematopoietic stem cells, human adult CD34+ cells from mobilized peripheral blood (mPB) were infused into nonirradiated NOD,B6.SCID Il2rγ−/− Kit(W41/W41) (“NBSGW”) mice. Briefly, CD34+ cells from mobilized peripheral blood (mPB) were prestimulated for two days with human cytokines and then electroporated with no RNP or with 8 μM of Sp35 RNP (comprising Sp35 gRNA (SEQ ID NO:339, Table 7) complexed with S. Pyogenes wildtype Cas9). Human chimerism and lineage reconstitution (erythroid, monocyte, neutrophil, B-cell, and HSPC reconstitution) in BM was analyzed 16 weeks after infusion (FIG. 19). These data suggest Sp35 RNP-edited CD34+ cells can successfully lead to long term engraftment in immunodeficient mice.

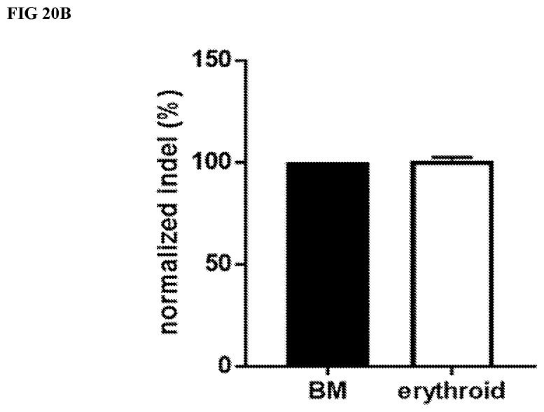

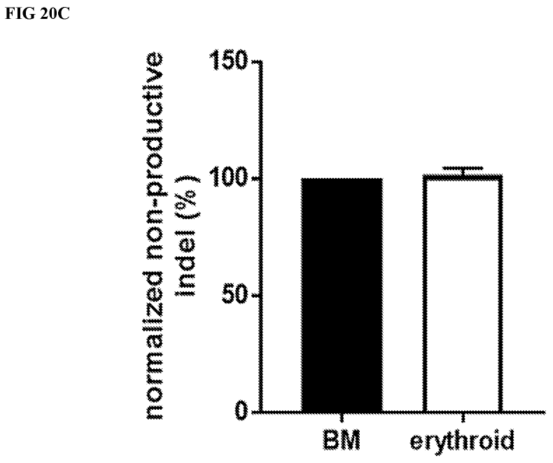

Next, total unfractionated BM or flow-sorted CD235a+ erythroid cells were extracted from the BM of the nonirradiated NBSGW mice 16 weeks after infusion of the modified mPB CD34+ cells. Indels in total unfractionated BM and in erythroid cells (FIG. 20A), indels in erythroid cells normalized to indels in corresponding BM (FIG. 20B), and non-productive indels, defined as deletions that disrupt less than 3 nt of the distal CCAAT box, in erythroid cells normalized to non-productive indels in corresponding BM (FIG. 20C) were determined. These data demonstrate that high levels of editing were achieved by Sp35 RNP in long term hematopoietic stem cells and editing patterns were comparable between erythroid cells and total BM.

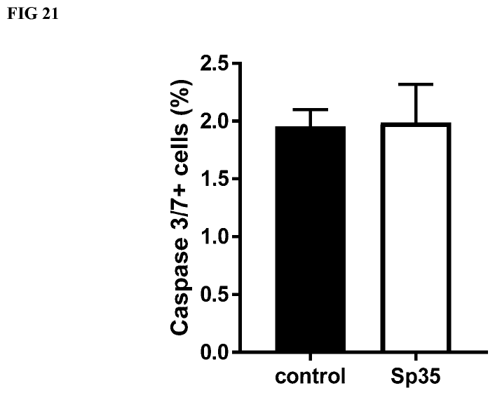

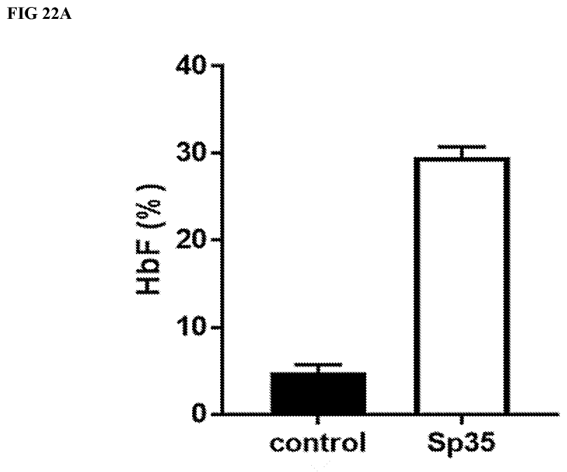

Next, the apoptosis status of cultured erythroid cells from the BM of the treated nonirradiated NBSGW mice was analyzed. Briefly, BM cells of the nonirradiated NBSGW mice 16 weeks after infusion of the modified mPB CD34+ cells were placed in erythroid culture media for 7 days and analyzed for caspase-3 and caspase-7 activity, a marker for apoptotic cells, using CellEvent™ Caspase-3/7 Green Flow Cytometry Assay Kit (FIG. 21). These data indicate that there was no significant induction of apoptosis in erythroid cells edited with Sp35 RNP.

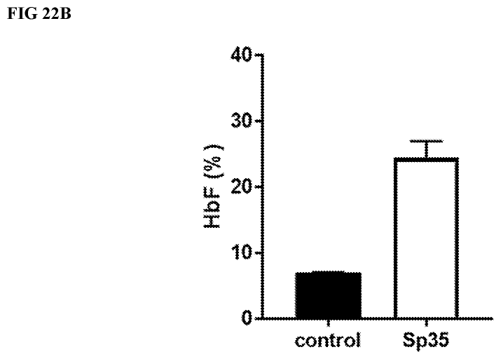

Lastly, long term HbF induction by Sp35 RNP edited erythroid cells was analyzed. Briefly, CD235a+ erythroid cells were obtained by flow sorting from chimeric BM 16 weeks after infusion of the modified mPB CD34+ cells. HbF expression, defined as gamma/beta-like chains, by the CD235a+ cells was determined (FIG. 22A). Erythroid progeny of engrafted human cells were obtained by culturing BM cells extracted from mice 16 weeks after infusion for 18 days in erythroid media. HbF expression by the ex-vivo derived erythroid progeny of engrafted human cells was determined (FIG. 22B). These data demonstrate that robust long-term HbF induction is achieved by Sp35 RNP editing of human CD34+ cells.

### Example 12: Treatment of β-Hemoglobinopathy Using Edited Hematopoietic Stem Cells

The methods and genome editing systems disclosed herein may be used for the treatment of a β-hemoglobinopathy, such as sickle cell disease or beta-thalassemia, in a patient in need thereof. For example, genome editing may be performed on cells derived from the patient in an autologous procedure. Correction of the patient's cells ex-vivo and reintroduction of the cells into the patient may result in increased HbF expression and treatment of the β-hemoglobinopathy.

For example, HSCs may be extracted from the bone marrow of a patient with a β-hemoglobinopathy using techniques that are well-known to skilled artisans. The HSCs may be modified using methods disclosed herein for genome editing. For example, RNPs comprised of guide RNAs (gRNAs) that target one or more regions in the HBG gene complexed with an RNA-guided nuclease may be used to edit the HSCs. In certain embodiments, the RNA-guided nuclease may be a Cas9 protein. In certain embodiments, the Cas9 protein may be a modified Cas9 protein. In certain embodiments, the gRNA may comprise one or more sequences set forth in Table 7. In certain embodiments, the gRNA may comprise one or more gRNAs comprising a targeting domain set forth in SEQ ID NOs:251-901, 940-942, 952-955, 970-979. In certain embodiments, modified HSCs have an increase in the frequency or level of an indel in the human HBG1 gene, HBG2 gene, or both, relative to unmodified HSCs. In certain embodiments, the modified HSCs can differentiate into erythroid cells that express an increased level of HbF. A population of the modified HSCs may be selected for reintroduction into the patient via transfusion or other methods known to skilled artisans. The population of modified HSCs for reintroduction may be selected based on, for example, increased HbF expression of the erythroid progeny of the modified HSCs or increased indel frequency of the modified HSCs. In some embodiments, any form of ablation prior to reintroduction of the cells may be used to enhance engraftment of the modified HSCs. In other embodiments, peripheral blood stem cells (PBSCs) can be extracted from a patient with a β-hemoglobinopathy using techniques that are well-known to skilled artisans (e.g., apheresis or leukapheresis) and stem cells can be removed from the PBSCs. The genome editing methods described above can be performed on the stem cells and the modified stem cells can be reintroduced into the patient as described above.

### Sequences

Genome editing system components according to the present disclosure (including without limitation, RNA-guided nucleases, guide RNAs, donor template nucleic acids, nucleic acids encoding nucleases or guide RNAs, and portions or fragments of any of the foregoing), are exemplified by the nucleotide and amino acid sequences presented in the Sequence Listing. The sequences presented in the Sequence Listing are not intended to be limiting, but rather illustrative of certain principles of genome editing systems and their component parts, which, in combination with the instant disclosure, will inform those of skill in the art about additional implementations and modifications that are within the scope of this disclosure. A list of the sequences presented is provided in the following Table 12.

TABLE 12
Sequences presented in the Sequence Listing:

| SEQ ID NOS: | DESCRIPTION |
|---|---|
| 1-2, 4-6, | Cas9 polypeptides |
| 12,14 | |
| 3, 7-11, 13 | Cas9 coding sequences |
| 15-23, | Cas9 RuvC-like domains |
| 52-123 | |
| 24-28, | Cas9 HNH-like domains |
| 124-198 | |
| 29-31, 38-51 | Full-length modular and |
| | unimolecular gRNAs |
| 32-37 | gRNA proximal and tail domains |
| 199-205 | PAM sequences |
| 251-901, 940- | gRNA targeting domains (RNA) - |
| 942, 952-955 | see Tables 2, 7, 9 |
| 910-919, 943- | gRNA targeting domains (DNA) - |
| 945, 956-959 | see Tables 7, 9 |
| 920-929, 946- | gRNA targeting domains plus |
| 948, 960-963 | PAM (NGG) (RNA) - see Tables |
| | 7, 9 |
| 930-939, 949- | gRNA targeting domains plus |
| 951, 964-967 | PAM (NGG) (DNA) - see Tables |
| | 7, 9 |
| 970-979 | Sp35 gRNA sequences (RNA) - |
| | see Table 10 |
| 980-989 | Sp35 gRNA sequences (DNA) - |
| | see Table 10 |
| 902, 903 | Human HBG1 and HBG2 |
| | promoter sequences including |
| | HPFH deletion site |
| 904-909, 990- | Oligonucleotide donor sequences |
| 995 | and homology arms - see Tables |
| | 8, 11 |
| 968-969 | BCL11Ae sequences |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

### REFERENCES

Ahern et al., Br J Haematol 25(4):437-444 (1973)
Akinbami Hemoglobin 40:64-65 (2016)
Aliyu et al. Am J Hematol 83:63-70 (2008)
Anders et al. Nature 513(7519):569-573 (2014)
Angastiniotis & Modell Ann N Y Acad Sci 850:251-269 (1998)
Bae et al. Bioinformatics 30(10):1473-1475 (2014)
Barbosa et al. Braz J Med Bio Res 43(8):705-711 (2010)
Bouva Hematologica 91(1):129-132 (2006)
Briner et al. Mol Cell 56(2):333-339 (2014)
Brousseau Am J Hematol 85(1):77-78 (2010)
Caldecott Nat Rev Genet 9(8):619-631 (2008)
Canvers et al. Nature 527(12):192-197 (2015)
Chang et al. Mol Ther Methods Clin Dev 4:137-148 (2017)
Chassanidis Ann Hematol 88(6):549-555 (2009)
Chylinski et al. RNA Biol 10(5):726-737 (2013)
Cong et al. Science 399(6121):819-823 (2013)
Costa et al., Cad Saude Publica 18(5):1469-1471 (2002)
Davis & Maizels 2 Proc Natl Acad Sci USA 111(10):E924-932 (2014)
Fine et al. Sci Rep. 5:10777 (2015)
Frit et al. DNA Repair (Amst) 17:81-97 (2014)
Fu et al. Nat Biotechnol 32(3):279-284 (2014)
Guilinger et al. Nat Biotechnol 32:577-582 (2014)
Heigwer et al. Nat Methods 11(2):122-123 (2014)
Hsu et al. Nat Biotechnol 31(9):827-832 (2013)
Iyama & Wilson DNA Repair (Amst) 12(8):620-636 (2013)
Jiang et al. Nat Biotechnol 31(3):233-239 (2013)
Jinek et al. Science 337(6096):816-821 (2012)
Jinek et al. Science 343(6176):1247997 (2014)
Kleinstiver et al. Nature 523(7561):481-485 (2015a)
Kleinstiver et al. Nat Biotechnol 33(12):1293-1298 (2015b)
Kleinstiver et al. Nature 529(7587):490-495 (2016)
Komor et al. Nature 533(7603):420-424 (2016)
Lee et al. Nano Lett 12(12):6322-6327 (2012)
Lewis “Medical-Surgical Nursing: Assessment and Management of Clinical Problems” (2014)
Li Cell Res 18(1):85-98 (2008)
Maeder et al. Int'l Patent Publication No. WO 2015/138510 (2015)
Makarova et al. Nat Rev Microbiol 9(6):467-477 (2011)
Mali et al. Science 339(6121):823-826 (2013)
Mantovani et al. Nucleic Acids Res 16(16):7783-7797 (1988)
Marteijn et al. Nat Rev Mol Cell Biol 15(7):465-481 (2014)
Nishimasu et al. Cell 156(5):935-949 (2014)
Nishimasu et al. Cell 162(5):1113-1126 (2015)
Ran & Hsu et al. Cell 154(6):1380-1389 (2013)
Richardson et al. Nat Biotechnol 34:339-344 (2016)
Shmakov et al. Molecular Cell 60(3):385-397 (2015)
Sternberg et al. Nature 507(7490):62-67 (2014)
Superti-Furga et al. EMBO J 7(10):3099-3107 (1988)
Thein Hum Mol Genet 18(R2):R216-223 (2009)
Tsai et al. Nat Biotechnol 34(5):483 (2016)
Waber et al. Blood 67(2):551-554 (1986)
Wang et al. Cell 153(4):910-918 (2013)
Xiao et al. Bioinformatics 30(8):1180-1182 (2014)
Xu et al. Genes Dev 24(8):783-798 (2010)
Yamano et al. Cell 165(4): 949-962 (2016)
Zetsche et al. Cell 163(3):759-771 (2015a)
Zetsche et al. Nat Biotechnol 33(2):139-42 (2015b)

1. A method of treating hemoglobinopathy in a subject in need thereof, the method comprising:
a) isolating a population of CD34+ or hematopoietic stem cells from the subject;
b) modifying the population of isolated cells ex vivo by delivering a first RNP complex comprising a first guide RNA (gRNA) and a first RNA-guided nuclease to the population of isolated cells, thereby to affect an alteration in a promoter of an HBG gene in one or more cells in the population; and
c) administering the modified population of cells to the subject, thereby to treat the hemoglobinopathy in the subject.

2. The method of claim 1, wherein the hemoglobinopathy is severe sickle cell disease (SCD) or thalassemia.

3. The method of claim 2, wherein the thalassemia is selected from the group consisting of β-thalassemia, δ-thalassemia, and β/δ-thalassemia.

4. The method of claim 1, wherein the RNA-guided nuclease is an S. pyogenes Cas9.

5. The method of claim 1, wherein the RNA-guided nuclease is a nickase, and optionally lacks RuvC activity.

6. The method of claim 5, wherein the first gRNA comprises a first targeting domain complimentary to a sequence within positions c. −1,114 to −114 of a human HBG1 or HBG2 gene.

7. The method of claim 5, wherein the first gRNA comprises a first targeting domain complimentary to a sequence within positions c. −214 to −114 of a human HBG1 or HBG2 gene.

8. The method of claim 1, wherein the first gRNA comprises a first targeting domain complimentary to a sequence within positions c. −102 to −52 of a human HBG1 or HBG2 gene.

9. The method of claim 1, wherein the first gRNA comprises a first targeting domain that differs by no more than 3 nucleotides from a targeting domain listed in Table 7.

10. The method of claim 1, further comprising delivering a second RNP complex comprising a second gRNA and a second RNA-guided nuclease to the population of isolated cells, thereby to affect an alteration in a promoter of an HBG gene in one or more cells in the population.

11. The method of claim 10, wherein the first and second gRNAs comprise first and second targeting domains complimentary to first and second sequences on opposite sides of positions of a 13 nt target region of a human HBG1 or HBG2 gene,
wherein one or both of the first and second sequences optionally overlaps the 13 nt target region of the human HBG1 or HBG2 gene.

12. An ex vivo method of increasing the level of fetal hemoglobin (HbF) in a human cell by genome editing using a first RNP complex comprising a first gRNA and a first RNA-guided nuclease to affect an alteration in a promoter of an HBG gene, thereby to increase expression of HbF.

13. The method of claim 12, wherein the first RNA-guided nuclease is an S. pyogenes Cas9.

14. The method of claim 12, wherein the first RNA-guided nuclease is a nickase, and optionally lacks RuvC activity.

15. The method of claim 12, wherein the first gRNA comprises a first targeting domain complimentary to a sequence within positions c. −1,114 to −114 of a human HBG1 or HBG2 gene.

16. The method of claim 12, wherein the first gRNA comprises a first targeting domain complimentary to a sequence within positions c. −214 to −114 of a human HBG1 or HBG2 gene.

17. The method of claim 12, wherein the first gRNA comprises a first targeting domain complimentary to a sequence within positions c. −102 to −52 of a human HBG1 or HBG2 gene.

18. The method of claim 12, wherein the first gRNA comprises a first targeting domain that differs by no more than 3 nucleotides from a targeting domain listed in Table 7.

19. The method of claim 12, further comprising using a second RNP complex comprising a second gRNA and a second RNA-guided nuclease to affect an alteration in a promoter of an HBG gene, thereby to increase expression of HbF.

20. The method of claim 19, wherein the first and second guide RNAs comprise first and second targeting domains complimentary to first and second sequences on opposite sides of positions of a 13 nt target region of a human HBG1 or HBG2 gene,
wherein one or both of the first and second sequences optionally overlaps the 13 nt target region of the human HBG1 or HBG2 gene.

21. A genome editing system, comprising:
an RNA-guided nuclease;
a first guide RNA; and
a second guide RNA,
wherein the first and second guide RNAs comprise first and second targeting domains complimentary to first and second sequences on opposite sides of positions of a 13 nt target region of a human HBG1 or HBG2 gene,
wherein one or both of the first and second sequences optionally overlaps the 13 nt target region of the human HBG1 or HBG2 gene.

22. The genome editing system of claim 21, further comprising a nucleic acid template encoding a deletion of the 13 nt region of a human HBG1 or HBG2 gene.

23. The genome editing system of claim 21, wherein the RNA-guided nuclease is an S. pyogenes Cas9.

24. The genome editing system of claim 21, wherein the first and second targeting domains are complimentary to sequences immediately adjacent to a protospacer adjacent motif recognized by S. pyogenes Cas9.

25. The genome editing system of claim 24, wherein the RNA-guided nuclease is a nickase, and optionally lacks RuvC activity.

26. The genome editing system of claim 21, wherein the first targeting domain is complimentary to a sequence within positions c. −1,114 to −114 of a human HBG1 or HBG2 gene.

27. The genome editing system of claim 21, wherein the first targeting domain is complimentary to a sequence within positions c. −214 to −114 of a human HBG1 or HBG2 gene.

28. The genome editing system of claim 21, wherein one of the first and second targeting domains is complimentary to a sequence within positions c. −102 to −52 of a human HBG1 or HBG2 gene.

29. The genome editing system of claim 21, wherein the second targeting domain is complimentary to a sequence within positions c. −102 to −2 of a human HBG1 or HBG2 gene.

30. The genome editing system of claim 21, wherein at least one of the first and second targeting domains differ by no more than 3 nucleotides from a targeting domain listed in Table 7.

31. The genome editing system of claim 21, comprising first and second RNA-guided nucleases.

32. The genome editing system of claim 21, wherein the first and second RNA-guided nucleases are complexed with the first and second guide RNAs, respectively, forming first and second ribonucleoprotein complexes.

33. The genome editing system of any one of claims 21-32, further comprising
a third guide RNA; and optionally
a fourth guide RNA,
wherein the third and fourth guide RNAs comprise third and fourth targeting domains complimentary to third and fourth sequences on opposite sides of positions of a GATA1 binding motif in BCL11A erythroid enhancer (BCL11Ae) of a human BCL11A gene,
wherein one or both of the third and fourth sequences optionally overlaps the GATA1 binding motif in BCL11Ae of the human BCL11A gene.

34. The genome editing system of claim 33, further comprising a nucleic acid template encoding a deletion of the GATA1 binding motif in BCL11Ae.

35. The genome editing system of claim 33, wherein the RNA-guided nuclease is an S. pyogenes Cas9.

36. The genome editing system of claim 33, wherein the third and fourth targeting domains are complimentary to sequences immediately adjacent to a protospacer adjacent motif recognized by S. pyogenes Cas9.

37. The genome editing system of claim 36, wherein the RNA-guided nuclease is a nickase, and optionally lacks RuvC activity.

38. The genome editing system of claim 33, wherein the third targeting domain is complimentary to a sequence within 1000 nucleotides upstream of the GATA1 binding motif in BCL11Ae.

39. The genome editing system of claim 33, wherein the third targeting domain is complimentary to a sequence within 100 nucleotides upstream of the GATA1 binding motif in BCL11Ae.

40. The genome editing system of claim 33, wherein one of the third and fourth targeting domains is complimentary to a sequence within 100 nucleotides downstream of the GATA1 binding motif in BCL11Ae.

41. The genome editing system of claim 33, wherein the fourth targeting domain is complimentary to a sequence within 50 nucleotides downstream of the GATA1 binding motif in BCL11Ae.

42. The genome editing system of claim 33, wherein at least one of the third and fourth targeting domains differ by no more than 3 nucleotides from a targeting domain listed in Table 9.

43. The genome editing system of claim 33, comprising first and second RNA-guided nucleases.

44. The genome editing system of claim 43, wherein the first and second RNA-guided nucleases are complexed with the third and fourth guide RNAs, respectively, forming third and fourth ribonucleoprotein complexes.

45. A method of altering a cell, comprising contacting a cell with the genome editing system of claim 29.

46. The method of claim 45, wherein the step of contacting the cell with the genome editing system comprises contacting the cell with a solution comprising first and second ribonucleoprotein complexes.

47. The method of claim 46, wherein the step of contacting the cell with the solution further comprises electroporating the cells, thereby introducing the first and second ribonucleoprotein complexes into the cell.

48. The method of claim 45, further comprising contacting the cell with the genome editing system of claim 41, wherein the step of contacting the cell with the genome editing system comprises contacting the cell with a solution comprising first, second, third, and optionally, fourth ribonucleoprotein complexes.

49. The method of claim 48, wherein the step of contacting the cell with the solution further comprises electroporating the cells, thereby introducing the first, second, third, and optionally, fourth ribonucleoprotein complexes into the cell.

50. The method of claim 45 or 48, wherein the cell is capable of differentiating into an erythroblast or a precursor of an erythroblast.

51. The method of claim 45 or 48, wherein the cell is capable of differentiating into an erythrocyte or a precursor of an erythrocyte.

52. The method of claim 45 or 48, wherein the cell is a CD34⁺ cell.

53. A CRISPR-mediated method of altering a cell, comprising:
introducing a first DNA single strand break (SSB) or double strand break (DSB) within a genome of the cell between positions c. −614 to −102 of a human HBG1 or HBG2 gene; and
introducing a second SSB or DSB within the genome of the cell between positions c. −114 to −1 of the human HBG1 or HBG2 gene,
wherein the first and second SSBs or DSBs are repaired by the cell in a manner that alters a 13 nt target region of the human HBG1 or HBG2 gene.

54. The CRISPR-mediated method of claim 53, wherein the first and second SSBs or DSBs are repaired by the cell in a manner that results in the deletion of all or part of a 13 nt target region of the human HBG1 or HBG2 gene.

55. The CRISPR-mediated method of claim 53, wherein the first and second SSBs or DSBs are repaired by the cell in a manner that results in the formation of at least one of an indel, a deletion, or an insertion in the 13 nt target region of the human HBG1 or HBG2 gene.

56. The CRISPR-mediated method of claim 53, wherein the first and second SSBs or DSBs are repaired by the cell in an error prone manner.

57. The CRISPR-mediated method of claim 53, further comprising
introducing a third DNA single strand break (SSB) or double strand break (DSB) within 500 nucleotides upstream of a GATA1 binding motif in BCL11Ae of a human BCL11Ae gene; and
introducing a fourth SSB or DSB within the genome of the cell within 100 nucleotides downstream of the GATA1 binding motif in BCL11Ae of the human BCL11Ae gene,
wherein the third and fourth SSBs or DSBs are repaired by the cell in a manner that alters the GATA1 binding motif in BCL11Ae of the human BCL11Ae gene.

58. The CRISPR-mediated method of claim 57, wherein the third and fourth SSBs or DSBs are repaired by the cell in a manner that results in the deletion of all or part of the GATA1 binding motif in BCL11Ae.

59. The CRISPR-mediated method of claim 57, wherein the third and fourth SSBs or DSBs are repaired by the cell in a manner that results in the formation of at least one of an indel, a deletion, or an insertion in the GATA1 binding motif in BCL11Ae.

60. The CRISPR-mediated method of claim 57, wherein the third and fourth SSBs or DSBs are repaired by the cell in an error prone manner.

61. A composition, comprising:
a plurality of cells generated by the method of claim 38, wherein at least 20%, 30%, 40%, 50%, 60%, 70%, 80% or 90% of the cells comprise an alteration of a sequence of a 13 nt target region of the human HBG1 or HBG2 gene or
a plurality of cells generated by the method of claim 57, wherein at least 20%, 30%, 40%, 50%, 60%, 70%, 80% or 90% of the cells comprise an alteration of a sequence of a 13 nt target region of the human HBG1 or HBG2 gene and at least 20%, 30%, 40%, 50%, 60%, 70%, 80% or 90% of the cells comprise an alteration of a sequence of the GATA1 binding motif in BCL11Ae.

62. The composition of claim 61, wherein at least a portion of the plurality of cells are within an erythroid lineage.

63. The composition of claim 62, wherein the plurality of cells is characterized by an increased level of fetal hemoglobin expression relative to an unmodified plurality of cells.

64. The composition of claim 63, wherein the level of fetal hemoglobin is increased by at least 20%, 30%, 40%, 50%, 60%, 70%, 80% or 90%.

65. The composition of claim 64, further comprising a pharmaceutically acceptable carrier.

66. A genome editing system, comprising:
an RNA-guided nuclease;
a guide RNA comprising a targeting domain targeting the 13 nt target region of a human HBG1 gene, HBG2 gene, or both.

67. The genome editing system of claim 66, wherein the RNA-guided nuclease is an S. pyogenes Cas9.

68. The genome editing system of claim 66 or 67, wherein the gRNA is in vitro transcribed or chemically synthesized.

69. The genome editing system of any one of claims 66-68, wherein the targeting domain differs by no more than 3 nucleotides from the sequence set forth in SEQ ID NO:339.

70. The genome editing system of any one of claims 66-69, wherein the gRNA differs by no more than 3 nucleotides from the sequence set forth in SEQ ID NO:970.

71. The genome editing system of claim 69 or 70, wherein the gRNA comprises a C-to-A sequence substitution at a 5′ end.

72. The genome editing system of claim 71, wherein the gRNA differs by no more than 3 nucleotides from the sequence set forth in SEQ ID NO:979.

73. The genome editing system of claim 70, wherein the gRNA comprises a truncated form of SEQ ID NO:970.

74. The genome editing system of claim 73, wherein the gRNA differs by no more than 3 nucleotides from the sequence set forth in SEQ ID NO:976-978.

75. The genome editing system of any one of claims 66-74, wherein the gRNA comprises one or more modifications.

76. The genome editing system of claim 75, wherein the one or more modifications are selected from the group consisting of phosphorothioate and 2′-O-methyl groups.

77. The genome editing system of claim 76, wherein the one or more modifications are at or near the 5′ end, 3′ end, or both of the gRNA.

78. The genome editing system of claim 77, wherein the gRNA comprises a sequence set forth in SEQ ID NOs:971-975.

79. The genome editing system of any one of claims 66-78, further comprising a template nucleic acid encoding a deletion of the 13 nt region of a human HBG1 or HBG2 gene.

80. The genome editing system of claim 79, wherein the template nucleic acid is a single stranded oligodeoxynucleotide (ssODN).

81. The genome editing system of claim 80, wherein the template nucleic acid is a positive strand or a negative strand.

82. The genome editing system of claim 81, wherein the ssODN comprises a 5′ homology arm, a replacement sequence, and a 3′ homology arm.

83. The genome editing system of claim 82, wherein the 5′ homology arm comprises about 200 nucleotides in length, e.g., at least 25, 50, 75, 100, 125, 150, 175, or 200 nucleotides in length; the replacement sequence comprises 0 nucleotides in length; and the 3′ homology arm comprises about 200 nucleotides in length, e.g., at least 25, 50, 75, 100, 125, 150, 175, or 200 nucleotides in length.

84. The genome editing system of claim 83, wherein the 5′ homology arm comprises about 50 to 100 bp, e.g., 55 to 95, 60 to 90, 70 to 90, or 80 to 90 bp, homology 5′ to the 13 nt target region and the 3′ homology arm comprises about 50 to 100 bp, e.g., 55 to 95, 60 to 90, 70 to 90, or 80 to 90 bp, homology 3′ to the 13 nt target region.

85. The genome editing system of claim 84, wherein the 13 nt target region is HBG1 c. −114 to −102 (e.g., nucleotides 2824-2836 of SEQ ID NO:902 (HBG1)), HBG2 c. −114 to −102 (e.g., nucleotides 2748-2760 of SEQ ID NO:903 (HBG2)), or a combination thereof.

86. The genome editing system of claim 85, wherein the 13 nt target region is HBG1 c. −114 to −102 (e.g., nucleotides 2824-2836 of SEQ ID NO:902 (HBG1)) and the 5′ homology arm comprises about 50 to 100 bp, e.g., 55 to 95, 60 to 90, 70 to 90, or 80 to 90 bp, homology 5′ of HBG1 c. −114 to −102 (e.g., nucleotides 2824-2836 of SEQ ID NO:902 (HBG1)).

87. The genome editing system of claim 86, wherein the 5′ homology arm comprises, consists essentially of, or consists of SEQ ID NO:904, SEQ ID NO:907, SEQ ID NO:991, or SEQ ID NO:994.

88. The genome editing system of claim 86 or 87, wherein the 3′ homology arm comprises about 50 to 100 bp, e.g., 55 to 95, 60 to 90, 70 to 90, or 80 to 90 bp, homology 3′ of HBG1 c. −114 to −102 (e.g., nucleotides 2824-2836 of SEQ ID NO:902 (HBG1)).

89. The genome editing system of any one of claims 86-88, wherein the 3′ homology arm comprises, consists essentially of, or consists of SEQ ID NO:905, SEQ ID NO:908, SEQ ID NO:992, or SEQ ID NO:995.

90. The genome editing system of claim 84, wherein the 13 nt target region is HBG2 c. −114 to −102 (e.g., nucleotides 2748-2760 of SEQ ID NO:903 (HBG2)) and the 5′ homology arm comprises about 50 to 100 bp, e.g., 55 to 95, 60 to 90, 70 to 90, or 80 to 90 bp, homology 5′ of HBG2 c. −114 to −102 (e.g., nucleotides 2748-2760 of SEQ ID NO:903 (HBG2)).

91. The genome editing system of claim 90, wherein the 5′ homology arm comprises, consists essentially of, or consists of SEQ ID NO:904, SEQ ID NO:907, SEQ ID NO:991, or SEQ ID NO:994.

92. The genome editing system of claim 90 or 91, wherein the 3′ homology arm comprises about 50 to 100 bp, e.g., 55 to 95, 60 to 90, 70 to 90, or 80 to 90 bp, homology 3′ of HBG2 c. −114 to −102 (e.g., nucleotides 2748-2760 of SEQ ID NO:903 (HBG2)).

93. The genome editing system of any one of claims 90-92, wherein the 3′ homology arm comprises, consists essentially of, or consists of SEQ ID NO:905, SEQ ID NO:908, SEQ ID NO:992, or SEQ ID NO:995.

94. The genome editing system of claim 84, wherein the ssODN comprises, consists essentially of, or consists of SEQ ID NO:906, SEQ ID NO:909, SEQ ID NO:990, or SEQ ID NO:995.

95. The genome editing system of any one of claims 80-93, wherein the ssODN comprises one or more phosphorothioate modifications at or near the 5′ end.

96. The genome editing system of any one of claims 81-94, wherein the ssODN comprises one or more phosphorothioate modifications at or near the 3′ end.

97. The genome editing system of any one of claims 81-94, wherein the ssODN comprises one or more phosphorothioate modifications at or near the 5′ end and 3′ end.

98. A method of altering a cell, comprising contacting a cell with the genome editing system of any one of claims 66-97.

99. The method of claim 98, wherein the cell is capable of differentiating into an erythroblast or a precursor of an erythroblast.

100. The method of claim 98, wherein the cell is capable of differentiating into an erythrocyte or a precursor of an erythrocyte.

101. The method of claim 98, wherein the cell is a CD34⁺ cell.


SEQUENCE LISTING (995 sequences)

SEQ ID NO: 1 - protein, 1345 aa, Streptococcus mutans
  misc_feature (10)..(21): N-terminal RuvC-like domain
  misc_feature (759)..(766): RuvC-like domain
  misc_feature (837)..(863): HNH-like domain
  misc_feature (982)..(989): RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - DNA, 4107 bp, Streptococcus pyogenes
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4 - protein, 1388 aa, Streptococcus thermophilus
  misc_feature (10)..(21): N-terminal RuvC-like domain
  misc_feature (760)..(767): RuvC-like domain
  misc_feature (844)..(870): HNH-like domain
  misc_feature (989)..(996): RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1334 aa, Listeria innocua
  misc_feature (10)..(21): N-terminal RuvC-like domain
  misc_feature (762)..(769): RuvC-like domain
  misc_feature (840)..(866): HNH-like domain
  misc_feature (985)..(992): RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 6 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - DNA, 3159 bp, Staphylococcus aureus
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Staphylococcus aureus
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Staphylococcus aureus
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10 - DNA, 3159 bp, Staphylococcus aureus
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 11 - DNA, 3159 bp, Staphylococcus aureus
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 12 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 13 - DNA, 3249 bp, Neisseria meningitidis
  misc_feature (1)..(3249): Exemplary codon optimized Cas9
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 14 - protein, 859 aa, Artificial Sequence
  Synthetic Cas9 consensus sequence derived from Sm, Sp, St, and Li Cas9 sequences
  misc_feature (4)..(4): Xaa can be any naturally occurring amino acid
  misc_feature (8)..(18): N-terminal RuvC-like domain
  misc_feature (21)..(21): Xaa can be any naturally occurring amino acid
  misc_feature (23)..(23): Xaa can be any naturally occurring amino acid
  misc_feature (24)..(24): Xaa can be any naturally occurring amino acid
  misc_feature (26)..(26): Xaa can be any naturally occurring amino acid
  misc_feature (29)..(31): Each Xaa can independently be any naturally occurring amino acid
  misc_feature (33)..(33): Xaa can be any naturally occurring amino acid
  misc_feature (36)..(36): Xaa can be any naturally occurring amino acid
  misc_feature (45)..(45): Xaa can be any naturally occurring amino acid
  misc_feature (54)..(54): Xaa can be any naturally occurring amino acid
  misc_feature (63)..(63): Xaa can be any naturally occurring amino acid
  misc_feature (71)..(71): Xaa can be any naturally occurring amino acid
  misc_feature (75)..(75): Xaa can be any naturally occurring amino acid
  misc_feature (76)..(76): Xaa can be any naturally occurring amino acid
  misc_feature (78)..(80): Each Xaa can independently be any naturally occurring amino acid
  misc_feature (82)..(82): Xaa can be any naturally occurring amino acid
  misc_feature (84)..(84): Xaa can be any naturally occurring amino acid
  misc_feature (85)..(85): Xaa can be any naturally occurring amino acid
  misc_feature (89)..(89): Xaa can be any naturally occurring amino acid
  misc_feature (90)..(90): Xaa can be any naturally occurring amino acid
  misc_feature (98)..(98): Xaa can be any naturally occurring amino acid
  misc_feature (100)..(100): Xaa can be any naturally occurring amino acid
  misc_feature (106)..(106): Xaa can be any naturally occurring amino acid
  misc_feature (113)..(113): Xaa can be any naturally occurring amino acid
  misc_feature (116)..(116): Xaa can be any naturally occurring amino acid
  misc_feature (125)..(125): Xaa can be any naturally occurring amino acid
  misc_feature (126)..(126): Xaa can be any naturally occurring amino acid
  misc_feature (128)..(133): Each Xaa can independently be any naturally occurring amino acid
  misc_feature (135)..(135): Xaa can be any naturally occurring amino acid
  misc_feature (137)..(137): Xaa can be any naturally occurring amino acid
  misc_feature (139)..(147): Each Xaa can independently be any naturally occurring amino acid
  misc_feature (153)..(155): Each Xaa can independently be any naturally occurring amino acid
  misc_feature (157)..(157): Xaa can be any naturally occurring amino acid
  misc_feature (159)..(159): Xaa can be any naturally occurring amino acid
  misc_feature (161)..(161): Xaa can be any naturally occurring amino acid
  misc_feature (163)..(163): Xaa can be any naturally occurring amino acid
  misc_feature (166)..(168): Each Xaa can independently be any naturally occurring amino acid
  misc_feature (170)..(170): Xaa can be any naturally occurring amino acid
  misc_feature (171)..(171): Xaa can be any naturally occurring amino acid
  misc_feature (173)..(175): Each Xaa can independently be any naturally occurring amino acid
  misc_feature (177)..(177): Xaa can be any naturally occurring amino acid
  misc_feature (183)..(183): Xaa can be any naturally occurring amino acid
  misc_feature (185)..(187): Each Xaa can independently be any naturally occurring amino acid
  misc_feature (189)..(189): Xaa can be any naturally occurring amino acid
  misc_feature (192)..(195): Each Xaa can independently be any naturally occurring amino acid
  misc_feature (198)..(198): Xaa can be any naturally occurring amino acid
  misc_feature (199)..(199): Xaa can be any naturally occurring amino acid
  misc_feature (202)..(202): Xaa can be any naturally occurring amino acid
  misc_feature (206)..(206): Xaa can be any naturally occurring amino acid
  misc_feature (207)..(207): Xaa can be any naturally occurring amino acid
  misc_feature (210)..(210): Xaa can be any naturally occurring amino acid
  misc_feature (212)..(212): Xaa can be any naturally occurring amino acid
  misc_feature (213)..(213): Xaa can be any naturally occurring amino acid
  misc_feature (219)..(219): Xaa can be any naturally occurring amino acid
  misc_feature (220)..(220): Xaa can be any naturally occurring amino acid
  misc_feature (222)..(222): Xaa can be any naturally occurring amino acid
  misc_feature (224)..(224): Xaa can be any naturally occurring amino acid
  misc_feature (226)..(226): Xaa can be any naturally occurring amino acid
  misc_feature (236)..(236): Xaa can be any naturally occurring amino acid
  misc_feature (240)..(240): Xaa can be any naturally occurring amino acid
  misc_feature (241)..(241): Xaa can be any naturally occurring amino acid
  misc_feature (244)..(246): Each Xaa can independently be any naturally occurring amino acid
  misc_feature (248)..(248): Xaa can be any naturally occurring amino acid
  misc_feature (249)..(249): Xaa can be any naturally occurring amino acid
  misc_feature (250)..(250): Xaa can be any naturally occurring amino acid
  misc_feature (252)..(254): Each Xaa can independently be any naturally occurring amino acid
  misc_feature (256)..(256): Xaa can be any naturally occurring amino acid
  misc_feature (257)..(257): Xaa can be any naturally occurring amino acid
  misc_feature (268)..(268): Xaa can be any naturally occurring amino acid
  misc_feature (271)..(271): Xaa can be any naturally occurring amino acid
  misc_feature (273)..(273): Xaa can be any naturally occurring amino acid
  misc_feature (277)..(277): Xaa can be any naturally occurring amino acid
  misc_feature (280)..(280): Xaa can be any naturally occurring amino acid
  misc_feature (281)..(281): Xaa can be any naturally occurring amino acid
  misc_feature (283)..(283): Xaa can be any naturally occurring amino acid
  misc_feature (289)..(289): Xaa can be any naturally occurring amino acid
  misc_feature (290)..(290): Xaa can be any naturally occurring amino acid
  misc_feature (292)..(294): Each Xaa can independently be any naturally occurring amino acid
  misc_feature (301)..(301): Xaa can be any naturally occurring amino acid
  misc_feature (308)..(308): Xaa can be any naturally occurring amino acid
  misc_feature (319)..(322): Each Xaa can independently be any naturally occurring amino acid
  misc_feature (328)..(328): Xaa can be any naturally occurring amino acid
  misc_feature (329)..(329): Xaa can be any naturally occurring amino acid
  misc_feature (335)..(337): Each Xaa can independently be any naturally occurring amino acid
  misc_feature (346)..(346): Xaa can be any naturally occurring amino acid
  misc_feature (347)..(347): Xaa can be any naturally occurring amino acid
  misc_feature (356)..(361): Each Xaa can independently be any naturally occurring amino acid
  misc_feature (363)..(363): Xaa can be any naturally occurring amino acid
  misc_feature (370)..(373): Each Xaa can independently be any naturally occurring amino acid
  misc_feature (375)..(375): Xaa can be any naturally occurring amino acid
  misc_feature (376)..(376): Xaa can be any naturally occurring amino acid
  misc_feature (379)..(379): Xaa can be any naturally occurring amino acid
  misc_feature (386)..(390): Each Xaa can independently be any naturally occurring amino acid
  misc_feature (393)..(393): Xaa can be any naturally occurring amino acid
  misc_feature (395)..(395): Xaa can be any naturally occurring amino acid
  misc_feature (396)..(396): Xaa can be any naturally occurring amino acid
  misc_feature (398)..(398): Xaa can be any naturally occurring amino acid
  misc_feature (400)..(400): Xaa can be any naturally occurring amino acid
  misc_feature (403)..(403): Xaa can be any naturally occurring amino acid
  misc_feature (407)..(407): Xaa can be any naturally occurring amino acid
  misc_feature (410)..(410): Xaa can be any naturally occurring amino acid
  misc_feature (411)..(411): Xaa can be any naturally occurring amino acid
  misc_feature (413)..(416): Each Xaa can independently be any naturally occurring amino acid
  misc_feature (418)..(418): Xaa can be any naturally occurring amino acid
  misc_feature (422)..(422): Xaa can be any naturally occurring amino acid
  misc_feature (428)..(428): Xaa can be any naturally occurring amino acid
  misc_feature (431)..(431): Xaa can be any naturally occurring amino acid
  misc_feature (433)..(433): Xaa can be any naturally occurring amino acid
  misc_feature (437)..(439): Each Xaa can independently be any naturally occurring amino acid
  misc_feature (445)..(445): Xaa can be any naturally occurring amino acid
  misc_feature (451)..(451): Xaa can be any naturally occurring amino acid
  misc_feature (456)..(456): Xaa can be any naturally occurring amino acid
  misc_feature (459)..(459): Xaa can be any naturally occurring amino acid
  misc_feature (465)..(469): Each Xaa can independently be any naturally occurring amino acid
  misc_feature (481)..(481): Xaa can be any naturally occurring amino acid
  misc_feature (482)..(482): Xaa can be any naturally occurring amino acid
  misc_feature (484)..(484): Xaa can be any naturally occurring amino acid
  misc_feature (490)..(490): Xaa can be any naturally occurring amino acid
  misc_feature (494)..(494): Xaa can be any naturally occurring amino acid
  misc_feature (495)..(502): RuvC-like domain
  misc_feature (497)..(497): Xaa can be any naturally occurring amino acid
  misc_feature (506)..(506): Xaa can be any naturally occurring amino acid
  misc_feature (510)..(510): Xaa can be any naturally occurring amino acid
  misc_feature (513)..(513): Xaa can be any naturally occurring amino acid
  misc_feature (514)..(514): Xaa can be any naturally occurring amino acid
  misc_feature (517)..(517): Xaa can be any naturally occurring amino acid
  misc_feature (520)..(520): Xaa can be any naturally occurring amino acid
  misc_feature (525)..(525): Xaa can be any naturally occurring amino acid
  misc_feature (526)..(526): Xaa can be any naturally occurring amino acid
  misc_feature (529)..(529): Xaa can be any naturally occurring amino acid
  misc_feature (531)..(531): Xaa can be any naturally occurring amino acid
  misc_feature (532)..(532): Xaa can be any naturally occurring amino acid
  misc_feature (534)..(534): Xaa can be any naturally occurring amino acid
  misc_feature (542)..(542): Xaa can be any naturally occurring amino acid
  misc_feature (546)..(546): Xaa can be any naturally occurring amino acid
  misc_feature (547)..(547): Xaa can be any naturally occurring amino acid
  misc_feature (553)..(553): Xaa can be any naturally occurring amino acid
  misc_feature (555)..(575): HNH-like domain
  misc_feature (556)..(556): Xaa can be any naturally occurring amino acid
  misc_feature (560)..(560): Xaa can be any naturally occurring amino acid
  misc_feature (563)..(563): Xaa can be any naturally occurring amino acid
  misc_feature (565)..(565): Xaa can be any naturally occurring amino acid
  misc_feature (567)..(567): Xaa can be any naturally occurring amino acid
  misc_feature (579)..(579): Xaa can be any naturally occurring amino acid
  misc_feature (582)..(582): Xaa can be any naturally occurring amino acid
  misc_feature (583)..(583): Xaa can be any naturally occurring amino acid
  misc_feature (585)..(585): Xaa can be any naturally occurring amino acid
  misc_feature (588)..(588): Xaa can be any naturally occurring amino acid
  misc_feature (590)..(590): Xaa can be any naturally occurring amino acid
  misc_feature (592)..(592): Xaa can be any naturally occurring amino acid
  misc_feature (594)..(596): Each Xaa can independently be any naturally occurring amino acid
  misc_feature (610)..(610): Xaa can be any naturally occurring amino acid
  misc_feature (616)..(616): Xaa can be any naturally occurring amino acid
  misc_feature (628)..(628): Xaa can be any naturally occurring amino acid
  misc_feature (631)..(631): Xaa can be any naturally occurring amino acid
  misc_feature (633)..(633): Xaa can be any naturally occurring amino acid
  misc_feature (634)..(634): Xaa can be any naturally occurring amino acid
  misc_feature (636)..(636): Xaa can be any naturally occurring amino acid
  misc_feature (638)..(641): Each Xaa can independently be any naturally occurring amino acid
  misc_feature (643)..(645): Each Xaa can independently be any naturally occurring amino acid
  misc_feature (653)..(653): Xaa can be any naturally occurring amino acid
  misc_feature (657)..(657): Xaa can be any naturally occurring amino acid
  misc_feature (659)..(659): Xaa can be any naturally occurring amino acid
  misc_feature (660)..(660): Xaa can be any naturally occurring amino acid
  misc_feature (665)..(665): Xaa can be any naturally occurring amino acid
  misc_feature (666)..(666): Xaa can be any naturally occurring amino acid
  misc_feature (668)..(668): Xaa can be any naturally occurring amino acid
  misc_feature (669)..(669): Xaa can be any naturally occurring amino acid
  misc_feature (670)..(677): RuvC-like domain
  misc_feature (680)..(680): Xaa can be any naturally occurring amino acid
  misc_feature (681)..(681): Xaa can be any naturally occurring amino acid
  misc_feature (683)..(683): Xaa can be any naturally occurring amino acid
  misc_feature (686)..(686): Xaa can be any naturally occurring amino acid
  misc_feature (696)..(696): Xaa can be any naturally occurring amino acid
  misc_feature (697)..(697): Xaa can be any naturally occurring amino acid
  misc_feature (704)..(704): Xaa can be any naturally occurring amino acid
  misc_feature (708)..(708): Xaa can be any naturally occurring amino acid
  misc_feature (710)..(710): Xaa can be any naturally occurring amino acid
  misc_feature (711)..(711): Xaa can be any naturally occurring amino acid
  misc_feature (714)..(714): Xaa can be any naturally occurring amino acid
  misc_feature (717)..(720): Each Xaa can independently be any naturally occurring amino acid
  misc_feature (722)..(722): Xaa can be any naturally occurring amino acid
  misc_feature (725)..(725): Xaa can be any naturally occurring amino acid
  misc_feature (727)..(727): Xaa can be any naturally occurring amino acid
  misc_feature (733)..(735): Each Xaa can independently be any naturally occurring amino acid
  misc_feature (740)..(740): Xaa can be any naturally occurring amino acid
  misc_feature (742)..(742): Xaa can be any naturally occurring amino acid
  misc_feature (749)..(754): Each Xaa can independently be any naturally occurring amino acid
  misc_feature (758)..(761): Each Xaa can independently be any naturally occurring amino acid
  misc_feature (763)..(768): Each Xaa can independently be any naturally occurring amino acid
  misc_feature (771)..(771): Xaa can be any naturally occurring amino acid
  misc_feature (774)..(777): Each Xaa can independently be any naturally occurring amino acid
  misc_feature (782)..(782): Xaa can be any naturally occurring amino acid
  misc_feature (784)..(786): Each Xaa can independently be any naturally occurring amino acid
  misc_feature (788)..(788): Xaa can be any naturally occurring amino acid
  misc_feature (790)..(790): Xaa can be any naturally occurring amino acid
  misc_feature (795)..(795): Xaa can be any naturally occurring amino acid
  misc_feature (799)..(799): Xaa can be any naturally occurring amino acid
  misc_feature (801)..(801): Xaa can be any naturally occurring amino acid
  misc_feature (802)..(802): Xaa can be any naturally occurring amino acid
  misc_feature (804)..(804): Xaa can be any naturally occurring amino acid
  misc_feature (806)..(813): Each Xaa can independently be any naturally occurring amino acid
  misc_feature (815)..(818): Each Xaa can independently be any naturally occurring amino acid
  misc_feature (820)..(820): Xaa can be any naturally occurring amino acid
  misc_feature (823)..(827): Each Xaa can independently be any naturally occurring amino acid
  misc_feature (829)..(829): Xaa can be any naturally occurring amino acid
  misc_feature (830)..(830): Xaa can be any naturally occurring amino acid
  misc_feature (832)..(832): Xaa can be any naturally occurring amino acid
  misc_feature (835)..(837): Each Xaa can independently be any naturally occurring amino acid
  misc_feature (842)..(844): Each Xaa can independently be any naturally occurring amino acid
  misc_feature (846)..(846): Xaa can be any naturally occurring amino acid
  misc_feature (848)..(848): Xaa can be any naturally occurring amino acid
  misc_feature (851)..(851): Xaa can be any naturally occurring amino acid
  misc_feature (857)..(857): Xaa can be any naturally occurring amino acid
  misc_feature (857)..(857): Xaa can be any naturally occurring amino acid
MKYXIGLDIGTNSVGWAVTDXYXXKXKGXXXIXKNXGLFDGTARXRTARRRRRXNRIYLQIFXEMDFFRLXSFVXXKXXXPXFXXEYHXXPTIYHLRXLXKDLRLXYLALAHXIKXRGNFLIEGXXNXXXXXXYXFXIXXXXXXXXXPEKGFXXXLXGXFXFXLEXXXKXXYXXXLXLLIGDXYXXXFXAKXXXXLSXXVTXALSXXMIXRXXHDLLKXXYXEXFXKGYAGYIDGXQFYXXKLXXXGXXXKXXXEXXLRKQRTFDNGXIPXQXHLEXAIXXQXYPFLNXXIXXXTFRIPYXVGPLAGXSFAWRKIPWNXXXXDSAFIXXMTDLPXXXVLPKHSLYXXVYNELTKVXXXXXXKXIFKRKVXXXXGXXFNXSTYHDLXXXXXLDXNXXEXIXLTXFEDXMIXXLXXXXKXLRRXYTGWGXLSXLXGIRXXXSTILDXLDNRNXMQLIXDLXFKIKQXXXXXGSPAIKKGILQXXKXVDELVXMGPXIVXEMARENQTXGNSXRKXXKEXGSXILKEXXNLXNXXLXLYYLQNGXDMYXXLDILSXYDXDHIXPQXFXDXSIDNVLSNRKDXVPXXVXKKXWXLXLXXXRKFDLTKAERGGLXDKAFIXRQLVETRQITKXVAXLXXNXDXXXXVXXXTLKSLVSXFRKXFXXLYKVXXNXXHHAHDAYLNVXXLXYPXLEEFVYGDYXXKATKFYXNIMXFXXGEXWKXXXXVXMQXNXVKKEQXXXPKNSXLXKDKYGGXXXXXXKGKXXXXIXXXXXXFLXGYXXXXLPKYXLXXXGXRXLASEXKGNXLXXLXAXXXXXXXXEXXXXFXANXXXXXLXXGXAFXXXIRRYXXXTXIXQSXTGLYEXRL

SEQ ID NO: 15 - protein, 8 aa, Artificial Sequence
  RuvC-like domain
  VARIANT (2)..(2): Xaa is Val or His
  VARIANT (3)..(3): Xaa is Ile, Leu, or Val
  VARIANT (5)..(5): Xaa is Met or Thr
IXXEXARE

SEQ ID NO: 16 - protein, 8 aa, Artificial Sequence
  RuvC-like domain
  VARIANT (3)..(3): Xaa is Ile, Leu, or Val
IVXEMARE

SEQ ID NO: 17 - protein, 8 aa, Artificial Sequence
  RuvC-like domain
  VARIANT (4)..(4): Xaa is His or Leu
  VARIANT (7)..(7): Xaa is Arg or Val
  VARIANT (8)..(8): Xaa is Glu or Val
HHAXDAXX

SEQ ID NO: 18 - protein, 8 aa, Artificial Sequence
  RuvC-like domain
HHAHDAYL

SEQ ID NO: 19 - protein, 30 aa, Artificial Sequence
  N-terminal RuvC-like domain
  VARIANT (2)..(2): Xaa is Lys or Pro
  VARIANT (4)..(4): Xaa is Val, Leu, Ile, or Phe
  VARIANT (5)..(5): Xaa is Gly, Ala, or Ser
  VARIANT (6)..(6): Xaa is Leu, Ile, Val, or Phe
  MOD_RES (7)..(26): N-terminal RuvC-like domain, each Xaa can be any amino acid or absent, region may encompass 5-20 residues
  VARIANT (29)..(29): Xaa is Asp, Glu, Asn, or Gln
KXYXXXXXXXXXXXXXXXXXXXXXXXTDXY

SEQ ID NO: 20 - protein, 12 aa, Artificial Sequence
  N-terminal RuvC-like domain
  VARIANT (2)..(2): Xaa is Ile, Val, Met, Leu, or Thr
  VARIANT (4)..(4): Xaa is Thr, Ile, Val, Ser, Asn, Tyr, Glu, or Leu
  VARIANT (5)..(5): Xaa is Asn, Ser, Gly, Ala, Asp, Thr, Arg, Met, or Phe
  VARIANT (6)..(6): Xaa is Ser, Tyr, Asn, or Phe
  VARIANT (7)..(7): Xaa is Val, Ile, Leu, Cys, Thr, or Phe
  VARIANT (9)..(9): Xaa is Trp, Phe, Val, Tyr, Ser, or Leu
  VARIANT (10)..(10): Xaa is Ala, Ser, Cys, Val, or Gly
  VARIANT (11)..(11): Xaa is Val, Ile, Leu, Ala, Met, or His
  VARIANT (12)..(12): Any amino acid or absent
DXGXXXXGXXXX

SEQ ID NO: 21 - protein, 12 aa, Artificial Sequence
  N-terminal RuvC-like domain
  VARIANT (2)..(2): Xaa is Ile, Val, Met, Leu, or Thr
  VARIANT (4)..(4): Xaa is Thr, Ile, Val, Ser, Asn, Tyr, Glu, or Leu
  VARIANT (5)..(5): Xaa is Asn, Ser, Gly, Ala, Asp, Thr, Arg, Met, or Phe
  VARIANT (7)..(7): Xaa is Val, Ile, Leu, Cys, Thr, or Phe
  VARIANT (9)..(9): Xaa is Trp, Phe, Val, Tyr, Ser, or Leu
  VARIANT (10)..(10): Xaa is Ala, Ser, Cys, Val, or Gly
  VARIANT (11)..(11): Xaa is Val, Ile, Leu, Ala, Met, or His
  VARIANT (12)..(12): Any amino acid or absent
DXGXXSXGXXXX

SEQ ID NO: 22 - protein, 12 aa, Artificial Sequence
  N-terminal RuvC-like domain
  VARIANT (4)..(4): Xaa is Thr, Ile, Val, Ser, Asn, Tyr, Glu, or Leu
  VARIANT (5)..(5): Xaa is Asn, Ser, Gly, Ala, Asp, Thr, Arg, Met, or Phe
  VARIANT (11)..(11): Xaa is Val, Ile, Leu, Ala, Met, or His
  VARIANT (12)..(12): Any amino acid or absent
DIGXXSVGWAXX

SEQ ID NO: 23 - protein, 12 aa, Artificial Sequence
  N-terminal RuvC-like domain
  MOD_RES (12)..(12): Any non-polar alkyl amino acid or a hydroxyl amino acid
DIGTNSVGWAVX

SEQ ID NO: 24 - protein, 73 aa, Artificial Sequence
  HNH-like domain
  VARIANT (8)..(8): Xaa is Lys or Arg
  VARIANT (12)..(12): Xaa is Val or Thr
  VARIANT (13)..(13): Xaa is Gly or Asp
  VARIANT (14)..(14): Xaa is Glu, Gln, or Asp
  VARIANT (15)..(15): Xaa is Glu or Asp
  VARIANT (19)..(19): Xaa is Asp, Asn, or His
  VARIANT (20)..(20): Xaa is Tyr, Arg, or Asn
  VARIANT (23)..(23): Xaa is Gln, Asp, or Asn
  MOD_RES (25)..(64): HNH-like domain, each Xaa can be any amino acid or absent, region may encompass 15-40 residues
  VARIANT (67)..(67): Xaa is Gly or Glu
  VARIANT (69)..(69): Xaa is Ser or Gly
  VARIANT (71)..(71): Xaa is Asp or Asn
LYYLQNGXDMYXXXXLDIXXLSXYXXXXXXXXXXXXXXXXXXXXXXXXXXXXXXXXXXXXXXXXNRXKXDXVP

SEQ ID NO: 25 - protein, 27 aa, Artificial Sequence
  HNH-like domain
  VARIANT (1)..(1): Xaa is Asp, Glu, Gln, or Asn
  VARIANT (2)..(2): Xaa is Leu, Ile, Arg, Gln, Val, Met, or Lys
  VARIANT (3)..(3): Xaa is Asp or Glu
  VARIANT (5)..(5): Xaa is Ile, Val, Thr, Ala, or Leu
  VARIANT (6)..(6): Xaa is Val, Tyr, Ile, Leu, Phe, or Trp
  VARIANT (8)..(8): Xaa is Gln, His, Arg, Lys, Tyr, Ile, Leu, Phe, or Trp
  VARIANT (9)..(9): Xaa is Ser, Ala, Asp, Thr, or Lys
  VARIANT (10)..(10): Xaa is Phe, Leu, Val, Lys, Tyr, Met, Ile, Arg, Ala, Glu, Asp, or Gln
  VARIANT (11)..(11): Xaa is Leu, Arg, Thr, Ile, Val, Ser, Cys, Tyr, Lys, Phe, or Gly
  VARIANT (12)..(12): Xaa is Lys, Gln, Tyr, Thr, Phe, Leu, Trp, Met, Ala, Glu, Gly, or Ser
  VARIANT (13)..(13): Xaa is Asp, Ser, Asn, Arg, Leu, or Thr
  VARIANT (14)..(14): Xaa is Asp, Asn, or Ser
  VARIANT (15)..(15): Xaa is Ser, Ala, Thr, Gly, or Arg
  VARIANT (16)..(16): Xaa is Ile, Leu, Phe, Ser, Arg, Tyr, Gln, Trp, Asp, Lys, or His
  VARIANT (17)..(17): Xaa is Asp, Ser, Ile, Asn, Glu, Ala, His, Phe, Leu, Gln, Met, Gly, Tyr, or Val
  VARIANT (19)..(19): Xaa is Lys, Leu, Arg, Met, Thr, or Phe
  VARIANT (20)..(20): Xaa is Val, Leu, Ile, Ala, or Thr
  VARIANT (21)..(21): Xaa is Leu, Ile, Val, or Ala
  VARIANT (22)..(22): Xaa is Thr, Val, Cys, Glu, Ser, or Ala
  VARIANT (23)..(23): Xaa is Arg, Phe, Thr, Trp, Glu, Leu, Asn, Cys, Lys, Val, Ser, Gln, Ile, Tyr, His, or Ala
  VARIANT (24)..(24): Xaa is Ser, Pro, Arg, Lys, Asn, Ala, His, Gln, Gly, or Leu
  VARIANT (25)..(25): Xaa is Asp, Gly, Thr, Asn, Ser, Lys, Ala, Ile, Glu, Leu, Gln, Arg, or Tyr
  VARIANT (26)..(26): Xaa is Lys, Val, Ala, Glu, Tyr, Ile, Cys, Leu, Ser, Thr, Gly, Lys, Met, Asp, or Phe
XXXHXXPXXXXXXXXXXNXXXXXXXXN

SEQ ID NO: 26 - protein, 27 aa, Artificial Sequence
  HNH-like domain
  VARIANT (1)..(1): Xaa is Asp or Glu
  VARIANT (2)..(2): Xaa is Leu, Ile, Arg, Gln, Val, Met, or Lys
  VARIANT (3)..(3): Xaa is Asp or Glu
  VARIANT (5)..(5): Xaa is Ile, Val, Thr, Ala, or Leu
  VARIANT (6)..(6): Xaa is Val, Tyr, Ile, Leu, Phe, or Trp
  VARIANT (8)..(8): Xaa is Gln, His, Arg, Lys, Tyr, Ile, Leu, Phe, or Trp
  VARIANT (10)..(10): Xaa is Phe, Leu, Val, Lys, Tyr, Met, Ile, Arg, Ala, Glu, Asp, or Gln
  VARIANT (11)..(11): Xaa is Leu, Arg, Thr, Ile, Val, Ser, Cys, Tyr, Lys, Phe, or Gly
  VARIANT (12)..(12): Xaa is Lys, Gln, Tyr, Thr, Phe, Leu, Trp, Met, Ala, Glu, Gly, or Ser
  VARIANT (16)..(16): Xaa is Ile, Leu, Phe, Ser, Arg, Tyr, Gln, Trp, Asp, Lys, or His
  VARIANT (17)..(17): Xaa is Asp, Ser, Ile, Asn, Glu, Ala, His, Phe, Leu, Gln, Met, Gly, Tyr, or Val
  VARIANT (22)..(22): Xaa is Thr, Val, Cys, Glu, Ser, or Ala
  VARIANT (23)..(23): Xaa is Arg, Phe, Thr, Trp, Glu, Leu, Asn, Cys, Lys, Val, Ser, Gln, Ile, Tyr, His, or Ala
  VARIANT (24)..(24): Xaa is Ser, Pro, Arg, Lys, Asn, Ala, His, Gln, Gly, or Leu
  VARIANT (25)..(25): Xaa is Asp, Gly, Thr, Asn, Ser, Lys, Ala, Ile, Glu, Leu, Gln, Arg, or Tyr
  VARIANT (26)..(26): Xaa is Lys, Val, Ala, Glu, Tyr, Ile, Cys, Leu, Ser, Thr, Gly, Lys, Met, Asp, or Phe
XXXHXXPXSXXXDDSXXNKVLXXXXXN

SEQ ID NO: 27 - protein, 27 aa, Artificial Sequence
  HNH-like domain
  VARIANT (1)..(1): Xaa is Asp or Glu
  VARIANT (3)..(3): Xaa is Asp or Glu
  VARIANT (8)..(8): Xaa is Gln, His, Arg, Lys, Tyr, Ile, Leu, or Trp
  VARIANT (10)..(10): Xaa is Phe, Leu, Val, Lys, Tyr, Met, Ile, Arg, Ala, Glu, Asp, or Gln
  VARIANT (11)..(11): Xaa is Leu, Arg, Thr, Ile, Val, Ser, Cys, Tyr, Lys, Phe, or Gly
  VARIANT (12)..(12): Xaa is Lys, Gln, Tyr, Thr, Phe, Leu, Trp, Met, Ala, Glu, Gly, or Ser
  VARIANT (16)..(16): Xaa is Ile, Leu, Phe, Ser, Arg, Tyr, Gln, Trp, Asp, Lys, or His
  VARIANT (17)..(17): Xaa is Asp, Ser, Ile, Asn, Glu, Ala, His, Phe, Leu, Gln, Met, Gly, Tyr, or Val
  VARIANT (23)..(23): Xaa is Arg, Phe, Thr, Trp, Glu, Leu, Asn, Cys, Lys, Val, Ser, Gln, Ile, Tyr, His, or Ala
  VARIANT (24)..(24): Xaa is Ser, Pro, Arg, Lys, Asn, Ala, His, Gln, Gly, or Leu
  VARIANT (25)..(25): Xaa is Asp, Gly, Thr, Asn, Ser, Lys, Ala, Ile, Glu, Leu, Gln, Arg, or Tyr
  VARIANT (26)..(26): Xaa is Lys, Val, Ala, Glu, Tyr, Ile, Cys, Leu, Ser, Thr, Gly, Lys, Met, Asp, or Phe
XVXHIVPXSXXXDDSXXNKVLTXXXXN

SEQ ID NO: 28 - protein, 27 aa, Artificial Sequence
  HNH-like domain
  VARIANT (2)..(2): Xaa is Ile or Val
  VARIANT (6)..(6): Xaa is Ile or Val
  VARIANT (9)..(9): Xaa is Ala or Ser
  VARIANT (11)..(11): Xaa is Ile or Leu
  VARIANT (12)..(12): Xaa is Lys or Thr
  VARIANT (14)..(14): Xaa is Asp or Asn
  VARIANT (19)..(19): Xaa is Arg, Lys, or Leu
  VARIANT (22)..(22): Xaa is Thr or Val
  VARIANT (23)..(23): Xaa is Ser or Arg
  VARIANT (25)..(25): Xaa is Lys, Asp, or Ala
  VARIANT (26)..(26): Xaa is Glu, Lys, Gly, or Asn
DXDHIXPQXFXXDXSIDNXVLXXSXXN

SEQ ID NO: 29 - RNA, 116 nt, Artificial Sequence
  gRNA
  misc_feature (1)..(20): a, c, u, g, unknown or other
  misc_feature (1)..(20): targeting region
  misc_feature (21)..(42): first complementarity domain
  misc_feature (43)..(46): linking domain
  misc_feature (47)..(70): second complementarity domain
nnnnnnnnnnnnnnnnnnnnguuuuagagcuaugcuguuuuggaaacaaaacagcauagcaaguuaaaauaaggcuaguccguuaucaacuugaaaaaguggcaccgagucggugc

SEQ ID NO: 30 - RNA, 116 nt, Artificial Sequence
  gRNA
  misc_feature (1)..(20): a, c, u, g, unknown or other
  misc_feature (1)..(20): targeting region
  misc_feature (21)..(42): first complementarity domain
  misc_feature (43)..(46): linking domain
  misc_feature (47)..(70): second complementarity domain
nnnnnnnnnnnnnnnnnnnnguauuagagcuaugcuguauuggaaacaauacagcauagcaaguuaauauaaggcuaguccguuaucaacuugaaaaaguggcaccgagucggugc

SEQ ID NO: 31 - RNA, 96 nt, Artificial Sequence
  gRNA
  misc_feature (1)..(20): a, c, u, g, unknown or other
  misc_feature (1)..(20): targeting domain
  misc_feature (21)..(32): first complementarity domain
  misc_feature (33)..(36): linking domain
  misc_feature (37)..(50): second complementarity domain
  misc_feature (51)..(62): proximal domain
nnnnnnnnnnnnnnnnnnnnguuuaagagcuagaaauagcaaguuuaaauaaggcuaguccguuaucaacuugaaaaaguggcaccgagucggugc

SEQ ID NO: 32 - RNA, 47 nt, Artificial Sequence
  gRNA proximal and tail domains derived from S. pyogenes
aaggcuaguccguuaucaacuugaaaaaguggcaccgagucggugcu

SEQ ID NO: 33 - RNA, 49 nt, Artificial Sequence
  gRNA proximal and tail domains
aaggcuaguccguuaucaacuugaaaaaguggcaccgagucgguggugc

SEQ ID NO: 34 - RNA, 51 nt, Artificial Sequence
  gRNA proximal and tail domains
aaggcuaguccguuaucaacuugaaaaaguggcaccgagucggugcggauc

SEQ ID NO: 35 - RNA, 31 nt, Artificial Sequence
  gRNA proximal and tail domains
aaggcuaguccguuaucaacuugaaaaagug

SEQ ID NO: 36 - RNA, 18 nt, Artificial Sequence
  gRNA proximal and tail domains
aaggcuaguccguuauca

SEQ ID NO: 37 - RNA, 12 nt, Artificial Sequence
  gRNA proximal and tail domains
aaggcuaguccg

SEQ ID NO: 38 - RNA, 102 nt, Artificial Sequence
  Unimolecular gRNA derived from S. aureus
  misc_feature (1)..(20): n is a, c, g, or u
  misc_feature (1)..(20): Targeting domain
nnnnnnnnnnnnnnnnnnnnguuuuaguacucuggaaacagaaucuacuaaaacaaggcaaaaugccguguuuaucucgucaacuuguuggcgagauuuuuu

SEQ ID NO: 39 - RNA, 42 nt, Artificial
  Modular gRNA derived from S. pyogenes
  misc_feature (1)..(20): a, c, u, g, unknown or other
  misc_feature (1)..(20): Targeting domain
  misc_feature (21)..(42): First complementarity domain
nnnnnnnnnnnnnnnnnnnnguuuuagagcuaugcuguuuug

SEQ ID NO: 40 - RNA, 85 nt, Artificial Sequence
  Modular gRNA derived from S. pyogenes
  misc_feature (1)..(9): 5′ extension domain
  misc_feature (10)..(33): Second complementarity domain
  misc_feature (34)..(45): Proximal domain
  misc_feature (46)..(85): Tail domain
ggaaccauucaaaacagcauagcaaguuaaaauaaggcuaguccguuaucaacuugaaaaaguggcaccgagucggugcuuuuuu

SEQ ID NO: 41 - RNA, 62 nt, Artificial Sequence
  Unimolecular gRNA derived from S. pyogenes
  misc_feature (1)..(20): a, c, u, g, unknown or other
  misc_feature (1)..(20): Targeting domain
  misc_feature (21)..(32): First complementarity domain
  misc_feature (33)..(36): Linking domain
  misc_feature (37)..(50): Second complementarity domain
  misc_feature (51)..(62): Proximal domain
nnnnnnnnnnnnnnnnnnnnguuuuagagcuagaaauagcaaguuaaaauaaggcuaguccg

SEQ ID NO: 42 - RNA, 102 nt, Artificial Sequence
  Unimolecular gRNA derived from S. pyogenes
  misc_feature (1)..(20): a, c, u, g, unknown or other
  misc_feature (1)..(20): Targeting domain
  misc_feature (21)..(32): First complementarity domain
  misc_feature (33)..(36): Linking domain
  misc_feature (37)..(50): Second complementarity domain
  misc_feature (51)..(62): Proximal domain
  misc_feature (63)..(102): Tail domain
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 43 - RNA, 75 nt, Artificial Sequence
  Unimolecular gRNA derived from S. pyogenes
  misc_feature (1)..(20): Targeting domain
  misc_feature (1)..(20): a, c, u, g, unknown or other
  misc_feature (21)..(36): First complementarity domain
  misc_feature (37)..(40): Linking domain
  misc_feature (41)..(58): Second complementarity domain
  misc_feature (59)..(70): Proximal domain
  misc_feature (71)..(75): Tail domain
nnnnnnnnnnnnnnnnnnnnguuuuagagcuaugcugaaaagcauagcaaguuaaaauaaggcuaguccguuauc

SEQ ID NO: 44 - RNA, 87 nt, Artificial Sequence
  Unimolecular gRNA derived from S. pyogenes
  misc_feature (1)..(20): a, c, u, g, unknown or other
  misc_feature (1)..(20): Targeting domain
  misc_feature (21)..(32): First complementarity domain
  misc_feature (43)..(46): Linking domain
  misc_feature (57)..(70): Second complementarity domain
  misc_feature (71)..(82): Proximal domain
  misc_feature (83)..(87): Tail domain
nnnnnnnnnnnnnnnnnnnnguuuuagagcuaugcuguuuuggaaacaaaacagcauagcaaguuaaaauaaggcuaguccguuauc

SEQ ID NO: 45 - RNA, 42 nt, Artificial Sequence
  Modular gRNA derived from S. thermophilus
  misc_feature (1)..(20): a, c, u, g, unknown or other
  misc_feature (1)..(20): Targeting domain
  misc_feature (21)..(42): First complementarity domain
nnnnnnnnnnnnnnnnnnnnguuuuagagcuguguuguuucg

SEQ ID NO: 46 - RNA, 78 nt, Artificial Sequence
  Modular gRNA derived from S. thermophilus
  misc_feature (1)..(3): 5′ extension domain
  misc_feature (4)..(27): Second complementarity domain
  misc_feature (28)..(40): Proximal domain
  misc_feature (41)..(78): Tail domain
gggcgaaacaacacagcgaguuaaaauaaggcuuaguccguacucaacuugaaaagguggcaccgauucgguguuuuu

SEQ ID NO: 47 - RNA, 85 nt, Artificial Sequence
  Modular gRNA derived from S. pyogenes
gaaccauucaaaacagcauagcaaguuaaaauaaggcuaguccguuaucaacuugaaaaaguggcaccgagucggugcuuuuuuu

SEQ ID NO: 48 - RNA, 96 nt, Artificial Sequence
  gRNA from S. pyogenes
  misc_feature (1)..(20): n is a, c, g, or u
  misc_feature (1)..(20): Targeting domain
nnnnnnnnnnnnnnnnnnnnguuuuagagcuagaaauagcaaguuaaaauaaggcuaguccguuaucaacuugaaaaaguggcaccgagucggugc

SEQ ID NO: 49 - RNA, 96 nt, Artificial Sequence
  gRNA
  misc_feature (1)..(20): n is a, c, g, or u
  misc_feature (1)..(20): Targeting domain
  misc_feature (21)..(32): First complementarity domain
  misc_feature (33)..(36): Linking domain
  misc_feature (37)..(50): Second complementarity domain
  misc_feature (51)..(62): Proximal domain
nnnnnnnnnnnnnnnnnnnnguauuagagcuagaaauagcaaguuaauauaaggcuaguccguuaucaacuugaaaaaguggcaccgagucggugc

SEQ ID NO: 50 - RNA, 104 nt, Artificial Sequence
  gRNA
  misc_feature (1)..(20): n is a, c, g, or u
  misc_feature (1)..(20): Targeting domain
  misc_feature (21)..(36): First complementarity domain
  misc_feature (37)..(40): Linking domain
  misc_feature (41)..(58): Second complementarity domain
nnnnnnnnnnnnnnnnnnnnguuuuagagcuaugcugaaaagcauagcaaguuaaaauaaggcuaguccguuaucaacuugaaaaaguggcaccgagucggugc

SEQ ID NO: 51 - RNA, 106 nt, Artificial Sequence
  gRNA
  misc_feature (1)..(20): n is a, c, g, or u
  misc_feature (1)..(20): Targeting domain
  misc_feature (21)..(37): First complementarity domain
  misc_feature (38)..(41): Linking domain
  misc_feature (42)..(60): Second complementarity domain
nnnnnnnnnnnnnnnnnnnnguuuuagagcuaugcuggaaacagcauagcaaguuaaaauaaggcuaguccguuaucaacuugaaaaaguggcaccgagucggugc

SEQ ID NO: 52 - protein, 12 aa, Peptoniphilus duerdenii
DIGTASVGWAVT

SEQ ID NO: 53 - protein, 12 aa, Treponema denticola
DVGTGSVGWAVT

SEQ ID NO: 54 - protein, 12 aa, S. mutans
DIGTNSVGWAVV

SEQ ID NO: 55 - protein, 12 aa, S. pyogenes
DIGTNSVGWAVI

SEQ ID NO: 56 - protein, 12 aa, L. innocua
DIGTNSVGWAVL

SEQ ID NO: 57 - protein, 12 aa, Flavobacterium branchiophilum FL-15
DLGTNSIGWAVV

SEQ ID NO: 58 - protein, 11 aa, Pedobacter glucosidilyticus
DLGTNSIGWAI

SEQ ID NO: 59 - protein, 12 aa, Bacteroides fragilis, NCTC 9343
DLGTNSIGWALV

SEQ ID NO: 60 - protein, 12 aa, Fusobacterium nucleatum
DIGTNSVGWCVT

SEQ ID NO: 61 - protein, 12 aa, Acidaminococcus sp. D21
DIGTNSVGYAVT

SEQ ID NO: 62 - protein, 12 aa, Coprococcus catus GD-7
DMGTGSLGWAVT

SEQ ID NO: 63 - protein, 12 aa, Oenococcus kitaharae DSM 17330
DIGTSSVGWAAI

SEQ ID NO: 64 - protein, 12 aa, Catenibacterium mitsuokai DSM 15897
DLGTGSVGWAVV

SEQ ID NO: 65 - protein, 12 aa, Mycoplasma gallisepticum str. F
DLGVGSVGWAIV

SEQ ID NO: 66 - protein, 12 aa, Mycoplasma ovipneumoniae SC01
DLGIASIGWAII

SEQ ID NO: 67 - protein, 12 aa, Mycoplasma canis PG 14
DLGIASVGWAIV

SEQ ID NO: 68 - protein, 12 aa, Mycoplasma synoviae 53
DLGVASVGWSIV

SEQ ID NO: 69 - protein, 12 aa, Eubacterium rectale
DIGIASVGWAIL

SEQ ID NO: 70 - protein, 12 aa, Enterococcus faecalis TX0012
DLGISSVGWSVI

SEQ ID NO: 71 - protein, 12 aa, Ilyobacter polytropus DSM 2926
DIGIASVGWSVI

SEQ ID NO: 72 - protein, 12 aa, Ruminococcus albus 8
DVGIGSIGWAVI

SEQ ID NO: 73 - protein, 12 aa, Elusimicrobium minutum Pei191
DLGVGSIGFAIV

SEQ ID NO: 74 - protein, 12 aa, Akkermansia muciniphila
DIGYASIGWAVI

SEQ ID NO: 75 - protein, 12 aa, Prevotella ruminicola
DTGTNSLGWAIV

SEQ ID NO: 76 - protein, 12 aa, Cand. Puniceispirillum marinum
DLGTNSIGWCLL

SEQ ID NO: 77 - protein, 12 aa, Rhodospirillum rubrum
DIGTDSLGWAVF

SEQ ID NO: 78 - protein, 12 aa, Lactobacillus rhamnosus GG
DIGSNSIGFAVV

SEQ ID NO: 79 - protein, 12 aa, Sphaerochaeta globus str. Buddy
DLGVGSIGVAVA

SEQ ID NO: 80 - protein, 12 aa, Rhodopseudomonas palustris
DLGIASCGWGVV

SEQ ID NO: 81 - protein, 12 aa, Mycoplasma mobile 163K
DLGIASVGWCLT

SEQ ID NO: 82 - protein, 12 aa, Streptococcus thermophilus LMD-9
DIGIGSVGVGIL

SEQ ID NO: 83 - protein, 12 aa, Staphylococcus lugdunensis M23590
DIGITSVGYGLI

SEQ ID NO: 84 - protein, 12 aa, Eubacterium dolichum DSM 3991
DIGITSVGFGII

SEQ ID NO: 85 - protein, 12 aa, Lactobacillus coryniformis KCTC 3535
DVGITSTGYAVL

SEQ ID NO: 86 - protein, 12 aa, Nitratifractor salsuginis DSM 16511
DLGITSFGYAIL

SEQ ID NO: 87 - protein, 12 aa, Bifidobacterium bifidum S17
DIGNASVGWSAF

SEQ ID NO: 88 - protein, 12 aa, Lactobacillus gasseri
DVGTNSCGWVAM

SEQ ID NO: 89 - protein, 12 aa, Acidothermus cellulolyticus 11B
DVGERSIGLAAV

SEQ ID NO: 90 - protein, 12 aa, Bifidobacterium longum DJO10A
DVGLNSVGLAAV

SEQ ID NO: 91 - protein, 12 aa, Bifidobacterium dentium
DVGLMSVGLAAI

SEQ ID NO: 92 - protein, 12 aa, Corynebacterium diphtheriae
DVGTFSVGLAAI

SEQ ID NO: 93 - protein, 12 aa, Staphylococcus pseudintermedius ED99
DIGTGSVGYACM

SEQ ID NO: 94 - protein, 12 aa, Capnocytophaga ochracea
DLGTTSIGFAHI

SEQ ID NO: 95 - protein, 12 aa, Prevotella denticola
DLGTNSIGSSVR

SEQ ID NO: 96 - protein, 12 aa, Ralstonia solanacearum
DIGTNSIGWAVI

SEQ ID NO: 97 - protein, 12 aa, Pasteurella multocida str. Pm70
DLGIASVGWAVV

SEQ ID NO: 98 - protein, 12 aa, Comamonas granuli
DIGIASVGWAVL

SEQ ID NO: 99 - protein, 12 aa, Helicobacter mustelae 12198
DIGIASIGWAVI

SEQ ID NO: 100 - protein, 12 aa, Agathobacter rectalis
DIGIASVGWAII

SEQ ID NO: 101 - protein, 12 aa, Clostridium cellulolyticum H10
DVGIASVGWAVI

SEQ ID NO: 102 - protein, 11 aa, Methylophilus sp. OH31
DIGIASVGWAL

SEQ ID NO: 103 - protein, 12 aa, Neisseria meningitidis
DIGIASVGWAMV

SEQ ID NO: 104 - protein, 12 aa, Clostridium perfringens
DIGITSVGWAVI

SEQ ID NO: 105 - protein, 12 aa, Wolinella succinogenes DSM 1740
DLGISSLGWAIV

SEQ ID NO: 106 - protein, 12 aa, Azospirillum sp. B510
DLGTNSIGWGLL

SEQ ID NO: 107 - protein, 12 aa, Verminephrobacter eiseniae
DLGSTSLGWAIF

SEQ ID NO: 108 - protein, 12 aa, Campylobacter jejuni, NCTC 11168
DIGISSIGWAFS

SEQ ID NO: 109 - protein, 12 aa, Parvibaculum lavamentivorans DS-1
DIGTTSIGFSVI

SEQ ID NO: 110 - protein, 12 aa, Dinoroseobacter shibae DFL 12
DIGTSSIGWWLY

SEQ ID NO: 111 - protein, 12 aa, Nitrobacter hamburgensis X14
DLGSNSLGWFVT

SEQ ID NO: 112 - protein, 12 aa, Bradyrhizobium sp. BTAi1
DLGANSLGWFVV

SEQ ID NO: 113 - protein, 15 aa, Bacillus cereus
DIGLRIGITSCGWSI

SEQ ID NO: 114 - protein, 12 aa, Sutterella wadsworthensis
DMGAKYTGVFYA

SEQ ID NO: 115 - protein, 12 aa, Wolinella succinogenes DSM 1740
DLGGKNTGFFSF

SEQ ID NO: 116 - protein, 12 aa, Francisella tularensis
DLGVKNTGVFSA

SEQ ID NO: 117 - protein, 12 aa, Gamma proteobacterium HTCC5015
DLGAKFTGVALY

SEQ ID NO: 118 - protein, 12 aa, Legionella pneumophila str. Paris
DLGGKFTGVCLS

SEQ ID NO: 119 - protein, 12 aa, Parasutterella excrementihominis
DLGGTYTGTFIT

SEQ ID NO: 120 - protein, 12 aa, S. thermophilus
DIGTNSVGWAVT

SEQ ID NO: 121 - protein, 12 aa, Eubacterium yurii
DVGTNSVGWAVT

SEQ ID NO: 122 - protein, 12 aa, Butyrivibrio hungatei
DMGTNSVGWAVT

SEQ ID NO: 123 - protein, 12 aa, Solobacterium moorei F0204
DVGTSSVGWAVT

SEQ ID NO: 124 - protein, 27 aa, Treponema denticola
DIDHIYPQSKIKDDSISNRVLVCSSCN

SEQ ID NO: 125 - protein, 27 aa, Coprococcus catus GD-7
DIDHIYPQSKTMDDSLNNRVLVKKNYN

SEQ ID NO: 126 - protein, 27 aa, Peptoniphilus duerdenii
DQDHIYPKSKIYDDSLENRVLVKKNLN

SEQ ID NO: 127 - protein, 27 aa, Catenibacterium mitsuokai DSM 15897
QIDHIVPQSLVKDDSFDNRVLVVPSEN

SEQ ID NO: 128 - protein, 27 aa, S. mutans
DIDHIIPQAFIKDNSIDNRVLTSSKEN

SEQ ID NO: 129 - protein, 27 aa, S. thermophilus
DIDHIIPQAFLKDNSIDNKVLVSSASN

SEQ ID NO: 130 - protein, 27 aa, Oenococcus kitaharae DSM 17330
DIDHIIPQAYTKDNSLDNRVLVSNITN

SEQ ID NO: 131 - protein, 27 aa, L. inocua
DIDHIVPQSFITDNSIDNLVLTSSAGN

SEQ ID NO: 132 - protein, 27 aa, S. pyogenes
DVDHIVPQSFLKDDSIDNKVLTRSDKN

SEQ ID NO: 133 - protein, 27 aa, Acidaminococcus sp. D21
NIDHIYPQSMVKDDSLDNKVLVQSEIN

SEQ ID NO: 134 - protein, 27 aa, Lactobacillus rhamnosus GG
DIDHILPQSLIKDDSLDNRVLVNATIN

SEQ ID NO: 135 - protein, 27 aa, Lactobacillus gasseri
DIDHILPQSFIKDDSLENRVLVKKAVN

SEQ ID NO: 136 - protein, 27 aa, Staphylococcus pseudintermedius ED99
EVDHIFPRSFIKDDSIDNKVLVIKKMN

SEQ ID NO: 137 - protein, 27 aa, Olsenella uli
EVDHIIPRSYIKDDSFENKVLVYREEN

SEQ ID NO: 138 - protein, 27 aa, Bifidobacterium bifidum S17
DIDHIIPQAVTQNDSIDNRVLVARAEN

SEQ ID NO: 139 - protein, 27 aa, Mycoplasma gallisepticum str. F
EIDHIIPYSISFDDSSSNKLLVLAESN

SEQ ID NO: 140 - protein, 27 aa, Mycoplasma canis PG 14
EIDHIIPYSLCFDDSSANKVLVHKQSN

SEQ ID NO: 141 - protein, 27 aa, Ilyobacter polytropus DSM 2926
DIDHIIPYSRSMDDSYSNKVLVLSGEN

SEQ ID NO: 142 - protein, 27 aa, Uncultured Termite group 1 bacterium
DIDHIIPYSKSMDDSFNNKVLCLAEEN

SEQ ID NO: 143 - protein, 27 aa, Campylobacter jejuni
EIDHIYPYSRSFDDSYMNKVLVFTKQN

SEQ ID NO: 144 - protein, 27 aa, Clostridium cellulolyticum H10
QIDHIYPYSRSMDDSYMNKVLVLTDEN

SEQ ID NO: 145 - protein, 27 aa, Clostridium perfringens
EIDHIIPFSRSFDDSLSNKILVLGSEN

SEQ ID NO: 146 - protein, 27 aa, N. meningitides
EIDHALPFSRTWDDSFNNKVLVLGSEN

SEQ ID NO: 147 - protein, 27 aa, Pasteurella multocida str. Pm70
EIDHALPFSRTWDDSFNNKVLVLASEN

SEQ ID NO: 148 - protein, 27 aa, Enterococcus faecalis TX0012
EIDHIIPISISLDDSINNKVLVLSKAN

SEQ ID NO: 149 - protein, 27 aa, Eubacterium dolichum DSM 3991
EVDHIIPISISLDDSITNKVLVTHREN

SEQ ID NO: 150 - protein, 27 aa, Acidovorax ebreus
QVDHALPYSRSYDDSKNNKVLVLTHEN

SEQ ID NO: 151 - protein, 27 aa, Streptococcus thermophilus LMD-9
EVDHILPLSITFDDSLANKVLVYATAN

SEQ ID NO: 152 - protein, 27 aa, Eubacterium rectale
EIDHIIPRSISFDDARSNKVLVYRSEN

SEQ ID NO: 153 - protein, 27 aa, Staphylococcus lugdunensis M23590
EVDHIIPRSVSFDNSYHNKVLVKQSEN

SEQ ID NO: 154 - protein, 27 aa, Roseburia intestinalis
DIDHILPYSITFDDSFRNKVLVTSQEN

SEQ ID NO: 155 - protein, 27 aa, Wolinella succinogenes DSM 1740
EIDHILPRSRSADDSFANKVLCLARAN

SEQ ID NO: 156 - protein, 27 aa, Cand. Puniceispirillum marinum
EIEHLLPFSLTLDDSMANKTVCFRQAN

SEQ ID NO: 157 - protein, 27 aa, Azospirillum sp. B510
DIDHILPFSVSLDDSAANKVVCLREAN

SEQ ID NO: 158 - protein, 27 aa, Bradyrhizobium sp. BTAi1
DIDHLIPFSISWDDSAANKVVCMRYAN

SEQ ID NO: 159 - protein, 27 aa, Nitrobacter hamburgensis X14
DIDHILPVAMTLDDSPANKIICMRYAN

SEQ ID NO: 160 - protein, 27 aa, Dinoroseobacter shibae
DVDHILPYSRTLDDSFPNRTLCLREAN

SEQ ID NO: 161 - protein, 27 aa, Verminephrobacter eiseniae
EIEHILPFSRTLDDSLNNRTVAMRRAN

SEQ ID NO: 162 - protein, 27 aa, Lactobacillus coryniformis KCTC 3535
EVDHIIPYSISWDDSYTNKVLTSAKCN

SEQ ID NO: 163 - protein, 27 aa, Rhodopseudomonas palustris
QVDHILPWSRFGDDSYLNKTLCTARSN

SEQ ID NO: 164 - protein, 27 aa, Ralstonia syzygii R24
QVDHILPFSKTLDDSFANKVLAQHDAN

SEQ ID NO: 165 - protein, 27 aa, Helicobacter mustelae 12198
QIDHAFPLSRSLDDSQSNKVLCLTSSN

SEQ ID NO: 166 - protein, 27 aa, Mycoplasma mobile 163K
DIDHIVPRSISFDDSFSNLVIVNKLDN

SEQ ID NO: 167 - protein, 27 aa, Mycoplasma ovipneumoniae SC01
EIEHIIPYSMSYDNSQANKILTEKAEN

SEQ ID NO: 168 - protein, 27 aa, Mycoplasma synoviae 53
EIDHVIPYSKSADDSWFNKLLVKKSTN

SEQ ID NO: 169 - protein, 27 aa, Aminomonas paucivorans DSM 12260
EMDHILPYSRSLDNGWHNRVLVHGKDN

SEQ ID NO: 170 - protein, 27 aa, Ruminococcus albus 8
EVDHIVPYSLILDNTINNKALVYAEEN

SEQ ID NO: 171 - protein, 27 aa, Fibrobacter succinogenes
EIEHVIPQSLYFDDSFSNKVICEAEVN

SEQ ID NO: 172 - protein, 27 aa, Bacteroides fragilis, NCTC 9343
DIEHIIPQARLFDDSFSNKTLEARSVN

SEQ ID NO: 173 - protein, 27 aa, Capnocytophaga sputigena
EIEHIVPKARVFDDSFSNKTLTFHRIN

SEQ ID NO: 174 - protein, 28 aa, Finegoldia magna
DKDHIIPQSMKKDDSIINNLVLVNKNAN

SEQ ID NO: 175 - protein, 27 aa, Parvibaculum lavamentivorans DS-1
EVEHIWPRSRSFDNSPRNKTLCRKDVN

SEQ ID NO: 176 - protein, 27 aa, Bacillus cereus
IVNHIIPYNRSFDDTYHNRVLTLTETK

SEQ ID NO: 177 - protein, 27 aa, Prevotella micans
DMEHTIPKSISFDNSDQNLTLCESYYN

SEQ ID NO: 178 - protein, 27 aa, Prevotella ruminicola
DIEHTIPRSAGGDSTKMNLTLCSSRFN

SEQ ID NO: 179 - protein, 27 aa, Flavobacterium columnare
DIEHTIPRSISQDNSQMNKTLCSLKFN

SEQ ID NO: 180 - protein, 27 aa, Rhodospirillum rubrum
DIDHVIPLARGGRDSLDNMVLCQSDAN

SEQ ID NO: 181 - protein, 27 aa, Elusimicrobium minutum Pei191
DIEHLFPIAESEDNGRNNLVISHSACN

SEQ ID NO: 182 - protein, 27 aa, Sphaerochaeta globus str. Buddy
DVDHIFPRDDTADNSYGNKVVAHRQCN

SEQ ID NO: 183 - protein, 27 aa, Nitratifractor salsuginis DSM 16511
DIEHIVPQSLGGLSTDYNTIVTLKSVN

SEQ ID NO: 184 - protein, 27 aa, Acidothermus cellulolyticus 11B
ELDHIVPRTDGGSNRHENLAITCGACN

SEQ ID NO: 185 - protein, 28 aa, Bifidobacterium longum DJO10A
EMDHIVPRKGVGSTNTRTNFAAVCAECN

SEQ ID NO: 186 - protein, 28 aa, Bifidobacterium dentium
EMDHIVPRKGVGSTNTRVNLAAACAACN

SEQ ID NO: 187 - protein, 28 aa, Corynebacterium diphtheriae
EMDHIVPRAGQGSTNTRENLVAVCHRCN

SEQ ID NO: 188 - protein, 33 aa, Sutterella wadsworthensis
EIDHILPRSLIKDARGIVFNAEPNLIYASSRGN

SEQ ID NO: 189 - protein, 33 aa, Gamma proteobacterium HTCC5015
EIDHIIPRSLTGRTKKTVFNSEANLIYCSSKGN

SEQ ID NO: 190 - protein, 33 aa, Parasutterella excrementihominis
EIDHIIPRSLTLKKSESIYNSEVNLIFVSAQGN

SEQ ID NO: 191 - protein, 33 aa, Legionella pneumophila str. Paris
EIDHIYPRSLSKKHFGVIFNSEVNLIYCSSQGN

SEQ ID NO: 192 - protein, 33 aa, Wolinella succinogenes DSM 1740
EIDHILPRSHTLKIYGTVFNPEGNLIYVHQKCN

SEQ ID NO: 193 - protein, 30 aa, Francisella tularensis
ELDHIIPRSHKKYGTLNDEANLICVTRGDN

SEQ ID NO: 194 - protein, 27 aa, Akkermansia muciniphila
ELEHIVPHSFRQSNALSSLVLTWPGVN

SEQ ID NO: 195 - protein, 27 aa, Solobacterium moorei F0204
DIDHIYPRSKIKDDSITNRVLVEKDIN

SEQ ID NO: 196 - protein, 28 aa, Veillonella atypica ACS-134-V-Col7a
YDIDHIYPRSLTKDDSFDNLVLCERTAN

SEQ ID NO: 197 - protein, 28 aa, Fusobacterium nucleatum
DIDHIYPRSKVIKDDSFDNLVLVLKNEN

SEQ ID NO: 198 - protein, 27 aa, Filifactor alocis
DRDHIYPQSKIKDDSIDNLVLVNKTYN

SEQ ID NO: 199 - DNA, 5 bp, Streptococcus thermophilus
  misc_feature (1)..(1): Any nucleotide (e.g., A, G, C, or T)
  misc_feature (4)..(4): Any nucleotide (e.g., A, G, C, or T)
nggng

SEQ ID NO: 200 - DNA, 7 bp, Streptococcus thermophilus
  misc_feature (1)..(1): Any nucleotide (e.g., A, G, C, or T)
  misc_feature (2)..(2): Any nucleotide (e.g., A, G, C, or T)
  misc_feature (7)..(7): A or T
nnagaaw

SEQ ID NO: 201 - DNA, 4 bp, Streptococcus mutans
  misc_feature (1)..(1): Any nucleotide (e.g., A, G, C, or T)
  misc_feature (4)..(4): A or G
naar

SEQ ID NO: 202 - DNA, 5 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
nngrr

SEQ ID NO: 203 - DNA, 6 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
  misc_feature (6)..(6): Any nucleotide (e.g., A, G, C, or T)
nngrrn

SEQ ID NO: 204 - DNA, 6 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
nngrrt

SEQ ID NO: 205 - DNA, 6 bp, Staphylococcus aureus
  misc_feature (1)..(1): Any nucleotide (e.g., A, G, C, or T)
  misc_feature (2)..(2): Any nucleotide (e.g., A, G, C, or T)
  misc_feature (4)..(4): A or G
  misc_feature (5)..(5): A or G
  misc_feature (6)..(6): A, G, or C
nngrrv

SEQ ID NO: 206-250 - skipped (45 entries)

SEQ ID NO: 251 - RNA, 17 nt, Artificial Sequence
  Targeting domain
gaaggaaacuagcuaaa

SEQ ID NO: 252 - RNA, 20 nt, Artificial Sequence
  Targeting domain
ggagaaggaaacuagcuaaa

SEQ ID NO: 253 - RNA, 20 nt, Artificial Sequence
  Targeting domain
gggagaaggaaacuagcuaa

SEQ ID NO: 254 - RNA, 20 nt, Artificial Sequence
  Targeting domain
guauccucuaugaugggaga

SEQ ID NO: 255 - RNA, 20 nt, Artificial Sequence
  Targeting domain
guuuccuucucccaucauag

SEQ ID NO: 256 - RNA, 20 nt, Artificial Sequence
  Targeting domain
guccugguauccucuaugau

SEQ ID NO: 257 - RNA, 17 nt, Artificial Sequence
  Targeting domain
agaaggaaacuagcuaa

SEQ ID NO: 258 - RNA, 17 nt, Artificial Sequence
  Targeting domain
uccucuaugaugggaga

SEQ ID NO: 259 - RNA, 17 nt, Artificial Sequence
  Targeting domain
ccugguauccucuauga

SEQ ID NO: 260 - RNA, 17 nt, Artificial Sequence
  Targeting domain
ccaucauagaggauacc

SEQ ID NO: 261 - RNA, 17 nt, Artificial Sequence
  Targeting domain
uccuucucccaucauag

SEQ ID NO: 262 - RNA, 17 nt, Artificial Sequence
  Targeting domain
uagcaguauccucuugg

SEQ ID NO: 263 - RNA, 17 nt, Artificial Sequence
  Targeting domain
uuagcaguauccucuug

SEQ ID NO: 264 - RNA, 17 nt, Artificial Sequence
  Targeting domain
aacuggaaugacugaau

SEQ ID NO: 265 - RNA, 17 nt, Artificial Sequence
  Targeting domain
cugguauccucuaugau

SEQ ID NO: 266 - RNA, 17 nt, Artificial Sequence
  Targeting domain
aauuagcaguauccucu

SEQ ID NO: 267 - RNA, 17 nt, Artificial Sequence
  Targeting domain
auuagcaguauccucuu

SEQ ID NO: 268 - RNA, 20 nt, Artificial Sequence
  Targeting domain
aguccugguauccucuauga

SEQ ID NO: 269 - RNA, 20 nt, Artificial Sequence
  Targeting domain
cucccaucauagaggauacc

SEQ ID NO: 270 - RNA, 20 nt, Artificial Sequence
  Targeting domain
aauuagcaguauccucuugg

SEQ ID NO: 271 - RNA, 20 nt, Artificial Sequence
  Targeting domain
aaauuagcaguauccucuug

SEQ ID NO: 272 - RNA, 20 nt, Artificial Sequence
  Targeting domain
aaaaacuggaaugacugaau

SEQ ID NO: 273 - RNA, 20 nt, Artificial Sequence
  Targeting domain
aaaaauuagcaguauccucu

SEQ ID NO: 274 - RNA, 20 nt, Artificial Sequence
  Targeting domain
aaaauuagcaguauccucuu

SEQ ID NO: 275 - RNA, 17 nt, Artificial Sequence
  Targeting domain
gaaucggaacaaggcaa

SEQ ID NO: 276 - RNA, 17 nt, Artificial Sequence
  Targeting domain
gaccaauagccuugaca

SEQ ID NO: 277 - RNA, 17 nt, Artificial Sequence
  Targeting domain
ggcuauuggucaaggca

SEQ ID NO: 278 - RNA, 17 nt, Artificial Sequence
  Targeting domain
gucaaggcuauugguca

SEQ ID NO: 279 - RNA, 17 nt, Artificial Sequence
  Targeting domain
guguguggaacugcuga

SEQ ID NO: 280 - RNA, 17 nt, Artificial Sequence
  Targeting domain
gggccggcggcuggcua

SEQ ID NO: 281 - RNA, 17 nt, Artificial Sequence
  Targeting domain
gaguauccagugaggcc

SEQ ID NO: 282 - RNA, 17 nt, Artificial Sequence
  Targeting domain
gcugacaaaagaagucc

SEQ ID NO: 283 - RNA, 17 nt, Artificial Sequence
  Targeting domain
ggccaggggccggcggc

SEQ ID NO: 284 - RNA, 17 nt, Artificial Sequence
  Targeting domain
gggaaggggcccccaag

SEQ ID NO: 285 - RNA, 17 nt, Artificial Sequence
  Targeting domain
gagauaguguggggaag

SEQ ID NO: 286 - RNA, 17 nt, Artificial Sequence
  Targeting domain
guauccagugaggccag

SEQ ID NO: 287 - RNA, 17 nt, Artificial Sequence
  Targeting domain
gugaggccaggggccgg

SEQ ID NO: 288 - RNA, 17 nt, Artificial Sequence
  Targeting domain
gcuggccaacccauggg

SEQ ID NO: 289 - RNA, 17 nt, Artificial Sequence
  Targeting domain
ggcuaaacuccacccau

SEQ ID NO: 290 - RNA, 17 nt, Artificial Sequence
  Targeting domain
ggauacucuaagacuau

SEQ ID NO: 291 - RNA, 17 nt, Artificial Sequence
  Targeting domain
ggggccggcggcuggcu

SEQ ID NO: 292 - RNA, 20 nt, Artificial Sequence
  Targeting domain
ggcuagggaugaagaauaaa

SEQ ID NO: 293 - RNA, 20 nt, Artificial Sequence
  Targeting domain
gaguguguggaacugcugaa

SEQ ID NO: 294 - RNA, 20 nt, Artificial Sequence
  Targeting domain
ggaaugacugaaucggaaca

SEQ ID NO: 295 - RNA, 20 nt, Artificial Sequence
  Targeting domain
gcauugagauagugugggga

SEQ ID NO: 296 - RNA, 20 nt, Artificial Sequence
  Targeting domain
gcuauuggucaaggcaaggc

SEQ ID NO: 297 - RNA, 20 nt, Artificial Sequence
  Targeting domain
guggggaaggggcccccaag

SEQ ID NO: 298 - RNA, 20 nt, Artificial Sequence
  Targeting domain
ggcaaggcuggccaacccau

SEQ ID NO: 299 - RNA, 20 nt, Artificial Sequence
  Targeting domain
guuugccuugucaaggcuau

SEQ ID NO: 300 - RNA, 20 nt, Artificial Sequence
  Targeting domain
gcuaaacuccacccaugggu

SEQ ID NO: 301 - RNA, 17 nt, Artificial Sequence
  Targeting domain
caaauaucugucugaaa

SEQ ID NO: 302 - RNA, 17 nt, Artificial Sequence
  Targeting domain
uagggaugaagaauaaa

SEQ ID NO: 303 - RNA, 17 nt, Artificial Sequence
  Targeting domain
ugagauaguguggggaa

SEQ ID NO: 304 - RNA, 17 nt, Artificial Sequence
  Targeting domain
uguguggaacugcugaa

SEQ ID NO: 305 - RNA, 17 nt, Artificial Sequence
  Targeting domain
augacugaaucggaaca

SEQ ID NO: 306 - RNA, 17 nt, Artificial Sequence
  Targeting domain
caaggcuggccaaccca

SEQ ID NO: 307 - RNA, 17 nt, Artificial Sequence
  Targeting domain
uggcuaaacuccaccca

SEQ ID NO: 308 - RNA, 17 nt, Artificial Sequence
  Targeting domain
uggguggaguuuagcca

SEQ ID NO: 309 - RNA, 17 nt, Artificial Sequence
  Targeting domain
aguauccagugaggcca

SEQ ID NO: 310 - RNA, 17 nt, Artificial Sequence
  Targeting domain
ucaaguuugccuuguca

SEQ ID NO: 311 - RNA, 17 nt, Artificial Sequence
  Targeting domain
uugagauagugugggga

SEQ ID NO: 312 - RNA, 17 nt, Artificial Sequence
  Targeting domain
auaaauuagagaaaaac

SEQ ID NO: 313 - RNA, 17 nt, Artificial Sequence
  Targeting domain
ccggccccuggccucac

SEQ ID NO: 314 - RNA, 17 nt, Artificial Sequence
  Targeting domain
agccagccgccggcccc

SEQ ID NO: 315 - RNA, 17 nt, Artificial Sequence
  Targeting domain
cugucugaaacgguccc

SEQ ID NO: 316 - RNA, 17 nt, Artificial Sequence
  Targeting domain
auggguggaguuuagcc

SEQ ID NO: 317 - RNA, 17 nt, Artificial Sequence
  Targeting domain
caucccuagccagccgc

SEQ ID NO: 318 - RNA, 17 nt, Artificial Sequence
  Targeting domain
auuggucaaggcaaggc

SEQ ID NO: 319 - RNA, 17 nt, Artificial Sequence
  Targeting domain
ccagugaggccaggggc

SEQ ID NO: 320 - RNA, 17 nt, Artificial Sequence
  Targeting domain
uuccacacacucgcuuc

SEQ ID NO: 321 - RNA, 17 nt, Artificial Sequence
  Targeting domain
cgcuucuggaacgucug

SEQ ID NO: 322 - RNA, 17 nt, Artificial Sequence
  Targeting domain
ucuuagaguauccagug

SEQ ID NO: 323 - RNA, 17 nt, Artificial Sequence
  Targeting domain
uuugcauugagauagug

SEQ ID NO: 324 - RNA, 17 nt, Artificial Sequence
  Targeting domain
uuccagaagcgagugug

SEQ ID NO: 325 - RNA, 17 nt, Artificial Sequence
  Targeting domain
ugcauugagauagugug

SEQ ID NO: 326 - RNA, 17 nt, Artificial Sequence
  Targeting domain
aaggcuggccaacccau

SEQ ID NO: 327 - RNA, 17 nt, Artificial Sequence
  Targeting domain
ugccuugucaaggcuau

SEQ ID NO: 328 - RNA, 17 nt, Artificial Sequence
  Targeting domain
aaacuccacccaugggu

SEQ ID NO: 329 - RNA, 17 nt, Artificial Sequence
  Targeting domain
uugcauugagauagugu

SEQ ID NO: 330 - RNA, 20 nt, Artificial Sequence
  Targeting domain
augcaaauaucugucugaaa

SEQ ID NO: 331 - RNA, 20 nt, Artificial Sequence
  Targeting domain
acugaaucggaacaaggcaa

SEQ ID NO: 332 - RNA, 20 nt, Artificial Sequence
  Targeting domain
cauugagauaguguggggaa

SEQ ID NO: 333 - RNA, 20 nt, Artificial Sequence
  Targeting domain
cuugaccaauagccuugaca

SEQ ID NO: 334 - RNA, 20 nt, Artificial Sequence
  Targeting domain
aggcaaggcuggccaaccca

SEQ ID NO: 335 - RNA, 20 nt, Artificial Sequence
  Targeting domain
cccuggcuaaacuccaccca

SEQ ID NO: 336 - RNA, 20 nt, Artificial Sequence
  Targeting domain
ccauggguggaguuuagcca

SEQ ID NO: 337 - RNA, 20 nt, Artificial Sequence
  Targeting domain
uagaguauccagugaggcca

SEQ ID NO: 338 - RNA, 20 nt, Artificial Sequence
  Targeting domain
caaggcuauuggucaaggca

SEQ ID NO: 339 - RNA, 20 nt, Artificial Sequence
  Targeting domain
cuugucaaggcuauugguca

SEQ ID NO: 340 - RNA, 20 nt, Artificial Sequence
  Targeting domain
uggucaaguuugccuuguca

SEQ ID NO: 341 - RNA, 20 nt, Artificial Sequence
  Targeting domain
cgaguguguggaacugcuga

SEQ ID NO: 342 - RNA, 20 nt, Artificial Sequence
  Targeting domain
caggggccggcggcuggcua

SEQ ID NO: 343 - RNA, 20 nt, Artificial Sequence
  Targeting domain
agaauaaauuagagaaaaac

SEQ ID NO: 344 - RNA, 20 nt, Artificial Sequence
  Targeting domain
ccgccggccccuggccucac

SEQ ID NO: 345 - RNA, 20 nt, Artificial Sequence
  Targeting domain
ccuagccagccgccggcccc

SEQ ID NO: 346 - RNA, 20 nt, Artificial Sequence
  Targeting domain
uaucugucugaaacgguccc

SEQ ID NO: 347 - RNA, 20 nt, Artificial Sequence
  Targeting domain
cccauggguggaguuuagcc

SEQ ID NO: 348 - RNA, 20 nt, Artificial Sequence
  Targeting domain
uuagaguauccagugaggcc

SEQ ID NO: 349 - RNA, 20 nt, Artificial Sequence
  Targeting domain
acggcugacaaaagaagucc

SEQ ID NO: 350 - RNA, 20 nt, Artificial Sequence
  Targeting domain
cuucaucccuagccagccgc

SEQ ID NO: 351 - RNA, 20 nt, Artificial Sequence
  Targeting domain
ugaggccaggggccggcggc

SEQ ID NO: 352 - RNA, 20 nt, Artificial Sequence
  Targeting domain
uauccagugaggccaggggc

SEQ ID NO: 353 - RNA, 20 nt, Artificial Sequence
  Targeting domain
caguuccacacacucgcuuc

SEQ ID NO: 354 - RNA, 20 nt, Artificial Sequence
  Targeting domain
auugagauaguguggggaag

SEQ ID NO: 355 - RNA, 20 nt, Artificial Sequence
  Targeting domain
agaguauccagugaggccag

SEQ ID NO: 356 - RNA, 20 nt, Artificial Sequence
  Targeting domain
ccagugaggccaggggccgg

SEQ ID NO: 357 - RNA, 20 nt, Artificial Sequence
  Targeting domain
aaggcuggccaacccauggg

SEQ ID NO: 358 - RNA, 20 nt, Artificial Sequence
  Targeting domain
acucgcuucuggaacgucug

SEQ ID NO: 359 - RNA, 20 nt, Artificial Sequence
  Targeting domain
uagucuuagaguauccagug

SEQ ID NO: 360 - RNA, 20 nt, Artificial Sequence
  Targeting domain
auauuugcauugagauagug

SEQ ID NO: 361 - RNA, 20 nt, Artificial Sequence
  Targeting domain
acguuccagaagcgagugug

SEQ ID NO: 362 - RNA, 20 nt, Artificial Sequence
  Targeting domain
auuugcauugagauagugug

SEQ ID NO: 363 - RNA, 20 nt, Artificial Sequence
  Targeting domain
ccuggcuaaacuccacccau

SEQ ID NO: 364 - RNA, 20 nt, Artificial Sequence
  Targeting domain
acuggauacucuaagacuau

SEQ ID NO: 365 - RNA, 20 nt, Artificial Sequence
  Targeting domain
ccaggggccggcggcuggcu

SEQ ID NO: 366 - RNA, 20 nt, Artificial Sequence
  Targeting domain
uauuugcauugagauagugu

SEQ ID NO: 367 - RNA, 19 nt, Artificial Sequence
  Targeting domain
guuuccuucucccaucaua

SEQ ID NO: 368 - RNA, 23 nt, Artificial Sequence
  Targeting domain
gcuaguuuccuucucccaucaua

SEQ ID NO: 369 - RNA, 19 nt, Artificial Sequence
  Targeting domain
gaauaaauuagagaaaaac

SEQ ID NO: 370 - RNA, 22 nt, Artificial Sequence
  Targeting domain
gaagaauaaauuagagaaaaac

SEQ ID NO: 371 - RNA, 23 nt, Artificial Sequence
  Targeting domain
ggaagaauaaauuagagaaaaac

SEQ ID NO: 372 - RNA, 24 nt, Artificial Sequence
  Targeting domain
gggaagaauaaauuagagaaaaac

SEQ ID NO: 373 - RNA, 20 nt, Artificial Sequence
  Targeting domain
gaaggaaacuagcuaaaggg

SEQ ID NO: 374 - RNA, 22 nt, Artificial Sequence
  Targeting domain
gagaaggaaacuagcuaaaggg

SEQ ID NO: 375 - RNA, 23 nt, Artificial Sequence
  Targeting domain
ggagaaggaaacuagcuaaaggg

SEQ ID NO: 376 - RNA, 24 nt, Artificial Sequence
  Targeting domain
gggagaaggaaacuagcuaaaggg

SEQ ID NO: 377 - RNA, 18 nt, Artificial Sequence
  Targeting domain
uuuccuucucccaucaua

SEQ ID NO: 378 - RNA, 20 nt, Artificial Sequence
  Targeting domain
aguuuccuucucccaucaua

SEQ ID NO: 379 - RNA, 21 nt, Artificial Sequence
  Targeting domain
uaguuuccuucucccaucaua

SEQ ID NO: 380 - RNA, 22 nt, Artificial Sequence
  Targeting domain
cuaguuuccuucucccaucaua

SEQ ID NO: 381 - RNA, 24 nt, Artificial Sequence
  Targeting domain
agcuaguuuccuucucccaucaua

SEQ ID NO: 382 - RNA, 18 nt, Artificial Sequence
  Targeting domain
agagaaaaacuggaauga

SEQ ID NO: 383 - RNA, 19 nt, Artificial Sequence
  Targeting domain
uagagaaaaacuggaauga

SEQ ID NO: 384 - RNA, 20 nt, Artificial Sequence
  Targeting domain
uuagagaaaaacuggaauga

SEQ ID NO: 385 - RNA, 21 nt, Artificial Sequence
  Targeting domain
auuagagaaaaacuggaauga

SEQ ID NO: 386 - RNA, 22 nt, Artificial Sequence
  Targeting domain
aauuagagaaaaacuggaauga

SEQ ID NO: 387 - RNA, 23 nt, Artificial Sequence
  Targeting domain
aaauuagagaaaaacuggaauga

SEQ ID NO: 388 - RNA, 24 nt, Artificial Sequence
  Targeting domain
uaaauuagagaaaaacuggaauga

SEQ ID NO: 389 - RNA, 18 nt, Artificial Sequence
  Targeting domain
aauaaauuagagaaaaac

SEQ ID NO: 390 - RNA, 21 nt, Artificial Sequence
  Targeting domain
aagaauaaauuagagaaaaac

SEQ ID NO: 391 - RNA, 18 nt, Artificial Sequence
  Targeting domain
aggaaacuagcuaaaggg

SEQ ID NO: 392 - RNA, 19 nt, Artificial Sequence
  Targeting domain
aaggaaacuagcuaaaggg

SEQ ID NO: 393 - RNA, 21 nt, Artificial Sequence
  Targeting domain
agaaggaaacuagcuaaaggg

SEQ ID NO: 394 - RNA, 18 nt, Artificial Sequence
  Targeting domain
uggggaaggggcccccaa

SEQ ID NO: 395 - RNA, 19 nt, Artificial Sequence
  Targeting domain
guggggaaggggcccccaa

SEQ ID NO: 396 - RNA, 20 nt, Artificial Sequence
  Targeting domain
uguggggaaggggcccccaa

SEQ ID NO: 397 - RNA, 21 nt, Artificial Sequence
  Targeting domain
guguggggaaggggcccccaa

SEQ ID NO: 398 - RNA, 22 nt, Artificial Sequence
  Targeting domain
aguguggggaaggggcccccaa

SEQ ID NO: 399 - RNA, 23 nt, Artificial Sequence
  Targeting domain
uaguguggggaaggggcccccaa

SEQ ID NO: 400 - RNA, 24 nt, Artificial Sequence
  Targeting domain
auaguguggggaaggggcccccaa

SEQ ID NO: 401 - RNA, 18 nt, Artificial Sequence
  Targeting domain
accucagacguuccagaa

SEQ ID NO: 402 - RNA, 19 nt, Artificial Sequence
  Targeting domain
aaccucagacguuccagaa

SEQ ID NO: 403 - RNA, 20 nt, Artificial Sequence
  Targeting domain
uaaccucagacguuccagaa

SEQ ID NO: 404 - RNA, 21 nt, Artificial Sequence
  Targeting domain
auaaccucagacguuccagaa

SEQ ID NO: 405 - RNA, 22 nt, Artificial Sequence
  Targeting domain
gauaaccucagacguuccagaa

SEQ ID NO: 406 - RNA, 23 nt, Artificial Sequence
  Targeting domain
ugauaaccucagacguuccagaa

SEQ ID NO: 407 - RNA, 24 nt, Artificial Sequence
  Targeting domain
uugauaaccucagacguuccagaa

SEQ ID NO: 408 - RNA, 18 nt, Artificial Sequence
  Targeting domain
cgccggccccuggccuca

SEQ ID NO: 409 - RNA, 19 nt, Artificial Sequence
  Targeting domain
ccgccggccccuggccuca

SEQ ID NO: 410 - RNA, 20 nt, Artificial Sequence
  Targeting domain
gccgccggccccuggccuca

SEQ ID NO: 411 - RNA, 21 nt, Artificial Sequence
  Targeting domain
agccgccggccccuggccuca

SEQ ID NO: 412 - RNA, 22 nt, Artificial Sequence
  Targeting domain
cagccgccggccccuggccuca

SEQ ID NO: 413 - RNA, 23 nt, Artificial Sequence
  Targeting domain
ccagccgccggccccuggccuca

SEQ ID NO: 414 - RNA, 24 nt, Artificial Sequence
  Targeting domain
gccagccgccggccccuggccuca

SEQ ID NO: 415 - RNA, 18 nt, Artificial Sequence
  Targeting domain
ggcaaggcuggccaaccc

SEQ ID NO: 416 - RNA, 19 nt, Artificial Sequence
  Targeting domain
aggcaaggcuggccaaccc

SEQ ID NO: 417 - RNA, 20 nt, Artificial Sequence
  Targeting domain
aaggcaaggcuggccaaccc

SEQ ID NO: 418 - RNA, 21 nt, Artificial Sequence
  Targeting domain
caaggcaaggcuggccaaccc

SEQ ID NO: 419 - RNA, 22 nt, Artificial Sequence
  Targeting domain
ucaaggcaaggcuggccaaccc

SEQ ID NO: 420 - RNA, 23 nt, Artificial Sequence
  Targeting domain
gucaaggcaaggcuggccaaccc

SEQ ID NO: 421 - RNA, 24 nt, Artificial Sequence
  Targeting domain
ggucaaggcaaggcuggccaaccc

SEQ ID NO: 422 - RNA, 18 nt, Artificial Sequence
  Targeting domain
ggcuggccaacccauggg

SEQ ID NO: 423 - RNA, 19 nt, Artificial Sequence
  Targeting domain
aggcuggccaacccauggg

SEQ ID NO: 424 - RNA, 21 nt, Artificial Sequence
  Targeting domain
caaggcuggccaacccauggg

SEQ ID NO: 425 - RNA, 22 nt, Artificial Sequence
  Targeting domain
gcaaggcuggccaacccauggg

SEQ ID NO: 426 - RNA, 23 nt, Artificial Sequence
  Targeting domain
ggcaaggcuggccaacccauggg

SEQ ID NO: 427 - RNA, 24 nt, Artificial Sequence
  Targeting domain
aggcaaggcuggccaacccauggg

SEQ ID NO: 428 - RNA, 18 nt, Artificial Sequence
  Targeting domain
gaguguguggaacugcug

SEQ ID NO: 429 - RNA, 19 nt, Artificial Sequence
  Targeting domain
cgaguguguggaacugcug

SEQ ID NO: 430 - RNA, 20 nt, Artificial Sequence
  Targeting domain
gcgaguguguggaacugcug

SEQ ID NO: 431 - RNA, 21 nt, Artificial Sequence
  Targeting domain
agcgaguguguggaacugcug

SEQ ID NO: 432 - RNA, 22 nt, Artificial Sequence
  Targeting domain
aagcgaguguguggaacugcug

SEQ ID NO: 433 - RNA, 23 nt, Artificial Sequence
  Targeting domain
gaagcgaguguguggaacugcug

SEQ ID NO: 434 - RNA, 24 nt, Artificial Sequence
  Targeting domain
agaagcgaguguguggaacugcug

SEQ ID NO: 435 - RNA, 18 nt, Artificial Sequence
  Targeting domain
ccuggcuaaacuccaccc

SEQ ID NO: 436 - RNA, 19 nt, Artificial Sequence
  Targeting domain
cccuggcuaaacuccaccc

SEQ ID NO: 437 - RNA, 20 nt, Artificial Sequence
  Targeting domain
ucccuggcuaaacuccaccc

SEQ ID NO: 438 - RNA, 21 nt, Artificial Sequence
  Targeting domain
gucccuggcuaaacuccaccc

SEQ ID NO: 439 - RNA, 22 nt, Artificial Sequence
  Targeting domain
ggucccuggcuaaacuccaccc

SEQ ID NO: 440 - RNA, 23 nt, Artificial Sequence
  Targeting domain
cggucccuggcuaaacuccaccc

SEQ ID NO: 441 - RNA, 24 nt, Artificial Sequence
  Targeting domain
acggucccuggcuaaacuccaccc

SEQ ID NO: 442 - RNA, 18 nt, Artificial Sequence
  Targeting domain
ggcggcuggcuagggaug

SEQ ID NO: 443 - RNA, 19 nt, Artificial Sequence
  Targeting domain
cggcggcuggcuagggaug

SEQ ID NO: 444 - RNA, 20 nt, Artificial Sequence
  Targeting domain
ccggcggcuggcuagggaug

SEQ ID NO: 445 - RNA, 21 nt, Artificial Sequence
  Targeting domain
gccggcggcuggcuagggaug

SEQ ID NO: 446 - RNA, 22 nt, Artificial Sequence
  Targeting domain
ggccggcggcuggcuagggaug

SEQ ID NO: 447 - RNA, 23 nt, Artificial Sequence
  Targeting domain
gggccggcggcuggcuagggaug

SEQ ID NO: 448 - RNA, 24 nt, Artificial Sequence
  Targeting domain
ggggccggcggcuggcuagggaug

SEQ ID NO: 449 - RNA, 18 nt, Artificial Sequence
  Targeting domain
aggggccggcggcuggcu

SEQ ID NO: 450 - RNA, 19 nt, Artificial Sequence
  Targeting domain
caggggccggcggcuggcu

SEQ ID NO: 451 - RNA, 21 nt, Artificial Sequence
  Targeting domain
gccaggggccggcggcuggcu

SEQ ID NO: 452 - RNA, 22 nt, Artificial Sequence
  Targeting domain
ggccaggggccggcggcuggcu

SEQ ID NO: 453 - RNA, 23 nt, Artificial Sequence
  Targeting domain
aggccaggggccggcggcuggcu

SEQ ID NO: 454 - RNA, 24 nt, Artificial Sequence
  Targeting domain
gaggccaggggccggcggcuggcu

SEQ ID NO: 455 - RNA, 18 nt, Artificial Sequence
  Targeting domain
aaacuugaccaauagucu

SEQ ID NO: 456 - RNA, 19 nt, Artificial Sequence
  Targeting domain
caaacuugaccaauagucu

SEQ ID NO: 457 - RNA, 20 nt, Artificial Sequence
  Targeting domain
gcaaacuugaccaauagucu

SEQ ID NO: 458 - RNA, 21 nt, Artificial Sequence
  Targeting domain
ggcaaacuugaccaauagucu

SEQ ID NO: 459 - RNA, 22 nt, Artificial Sequence
  Targeting domain
aggcaaacuugaccaauagucu

SEQ ID NO: 460 - RNA, 23 nt, Artificial Sequence
  Targeting domain
aaggcaaacuugaccaauagucu

SEQ ID NO: 461 - RNA, 24 nt, Artificial Sequence
  Targeting domain
caaggcaaacuugaccaauagucu

SEQ ID NO: 462 - RNA, 18 nt, Artificial Sequence
  Targeting domain
uucagacagauauuugca

SEQ ID NO: 463 - RNA, 19 nt, Artificial Sequence
  Targeting domain
uuucagacagauauuugca

SEQ ID NO: 464 - RNA, 20 nt, Artificial Sequence
  Targeting domain
guuucagacagauauuugca

SEQ ID NO: 465 - RNA, 21 nt, Artificial Sequence
  Targeting domain
cguuucagacagauauuugca

SEQ ID NO: 466 - RNA, 22 nt, Artificial Sequence
  Targeting domain
ccguuucagacagauauuugca

SEQ ID NO: 467 - RNA, 23 nt, Artificial Sequence
  Targeting domain
accguuucagacagauauuugca

SEQ ID NO: 468 - RNA, 24 nt, Artificial Sequence
  Targeting domain
gaccguuucagacagauauuugca

SEQ ID NO: 469 - RNA, 18 nt, Artificial Sequence
  Targeting domain
aguuuccuucucccauca

SEQ ID NO: 470 - RNA, 19 nt, Artificial Sequence
  Targeting domain
uaguuuccuucucccauca

SEQ ID NO: 471 - RNA, 20 nt, Artificial Sequence
  Targeting domain
cuaguuuccuucucccauca

SEQ ID NO: 472 - RNA, 21 nt, Artificial Sequence
  Targeting domain
gcuaguuuccuucucccauca

SEQ ID NO: 473 - RNA, 22 nt, Artificial Sequence
  Targeting domain
agcuaguuuccuucucccauca

SEQ ID NO: 474 - RNA, 23 nt, Artificial Sequence
  Targeting domain
uagcuaguuuccuucucccauca

SEQ ID NO: 475 - RNA, 24 nt, Artificial Sequence
  Targeting domain
uuagcuaguuuccuucucccauca

SEQ ID NO: 476 - RNA, 18 nt, Artificial Sequence
  Targeting domain
auugagauagugugggga

SEQ ID NO: 477 - RNA, 19 nt, Artificial Sequence
  Targeting domain
cauugagauagugugggga

SEQ ID NO: 478 - RNA, 21 nt, Artificial Sequence
  Targeting domain
ugcauugagauagugugggga

SEQ ID NO: 479 - RNA, 22 nt, Artificial Sequence
  Targeting domain
uugcauugagauagugugggga

SEQ ID NO: 480 - RNA, 23 nt, Artificial Sequence
  Targeting domain
uuugcauugagauagugugggga

SEQ ID NO: 481 - RNA, 24 nt, Artificial Sequence
  Targeting domain
auuugcauugagauagugugggga

SEQ ID NO: 482 - RNA, 18 nt, Artificial Sequence
  Targeting domain
ucccaucauagaggauac

SEQ ID NO: 483 - RNA, 19 nt, Artificial Sequence
  Targeting domain
cucccaucauagaggauac

SEQ ID NO: 484 - RNA, 20 nt, Artificial Sequence
  Targeting domain
ucucccaucauagaggauac

SEQ ID NO: 485 - RNA, 21 nt, Artificial Sequence
  Targeting domain
uucucccaucauagaggauac

SEQ ID NO: 486 - RNA, 22 nt, Artificial Sequence
  Targeting domain
cuucucccaucauagaggauac

SEQ ID NO: 487 - RNA, 23 nt, Artificial Sequence
  Targeting domain
ccuucucccaucauagaggauac

SEQ ID NO: 488 - RNA, 24 nt, Artificial Sequence
  Targeting domain
uccuucucccaucauagaggauac

SEQ ID NO: 489 - RNA, 18 nt, Artificial Sequence
  Targeting domain
uguggggaaggggccccc

SEQ ID NO: 490 - RNA, 19 nt, Artificial Sequence
  Targeting domain
guguggggaaggggccccc

SEQ ID NO: 491 - RNA, 20 nt, Artificial Sequence
  Targeting domain
aguguggggaaggggccccc

SEQ ID NO: 492 - RNA, 21 nt, Artificial Sequence
  Targeting domain
uaguguggggaaggggccccc

SEQ ID NO: 493 - RNA, 22 nt, Artificial Sequence
  Targeting domain
auaguguggggaaggggccccc

SEQ ID NO: 494 - RNA, 23 nt, Artificial Sequence
  Targeting domain
gauaguguggggaaggggccccc

SEQ ID NO: 495 - RNA, 24 nt, Artificial Sequence
  Targeting domain
agauaguguggggaaggggccccc

SEQ ID NO: 496 - RNA, 18 nt, Artificial Sequence
  Targeting domain
cauggguggaguuuagcc

SEQ ID NO: 497 - RNA, 19 nt, Artificial Sequence
  Targeting domain
ccauggguggaguuuagcc

SEQ ID NO: 498 - RNA, 21 nt, Artificial Sequence
  Targeting domain
acccauggguggaguuuagcc

SEQ ID NO: 499 - RNA, 22 nt, Artificial Sequence
  Targeting domain
aacccauggguggaguuuagcc

SEQ ID NO: 500 - RNA, 23 nt, Artificial Sequence
  Targeting domain
caacccauggguggaguuuagcc

SEQ ID NO: 501 - RNA, 24 nt, Artificial Sequence
  Targeting domain
ccaacccauggguggaguuuagcc

SEQ ID NO: 502 - RNA, 18 nt, Artificial Sequence
  Targeting domain
ccauggguggaguuuagc

SEQ ID NO: 503 - RNA, 19 nt, Artificial Sequence
  Targeting domain
cccauggguggaguuuagc

SEQ ID NO: 504 - RNA, 20 nt, Artificial Sequence
  Targeting domain
acccauggguggaguuuagc

SEQ ID NO: 505 - RNA, 21 nt, Artificial Sequence
  Targeting domain
aacccauggguggaguuuagc

SEQ ID NO: 506 - RNA, 22 nt, Artificial Sequence
  Targeting domain
caacccauggguggaguuuagc

SEQ ID NO: 507 - RNA, 23 nt, Artificial Sequence
  Targeting domain
ccaacccauggguggaguuuagc

SEQ ID NO: 508 - RNA, 24 nt, Artificial Sequence
  Targeting domain
gccaacccauggguggaguuuagc

SEQ ID NO: 509 - RNA, 18 nt, Artificial Sequence
  Targeting domain
ugauaaccucagacguuc

SEQ ID NO: 510 - RNA, 19 nt, Artificial Sequence
  Targeting domain
uugauaaccucagacguuc

SEQ ID NO: 511 - RNA, 20 nt, Artificial Sequence
  Targeting domain
auugauaaccucagacguuc

SEQ ID NO: 512 - RNA, 21 nt, Artificial Sequence
  Targeting domain
uauugauaaccucagacguuc

SEQ ID NO: 513 - RNA, 22 nt, Artificial Sequence
  Targeting domain
uuauugauaaccucagacguuc

SEQ ID NO: 514 - RNA, 23 nt, Artificial Sequence
  Targeting domain
cuuauugauaaccucagacguuc

SEQ ID NO: 515 - RNA, 24 nt, Artificial Sequence
  Targeting domain
gcuuauugauaaccucagacguuc

SEQ ID NO: 516 - RNA, 18 nt, Artificial Sequence
  Targeting domain
cauugagauagugugggg

SEQ ID NO: 517 - RNA, 19 nt, Artificial Sequence
  Targeting domain
gcauugagauagugugggg

SEQ ID NO: 518 - RNA, 20 nt, Artificial Sequence
  Targeting domain
ugcauugagauagugugggg

SEQ ID NO: 519 - RNA, 21 nt, Artificial Sequence
  Targeting domain
uugcauugagauagugugggg

SEQ ID NO: 520 - RNA, 22 nt, Artificial Sequence
  Targeting domain
uuugcauugagauagugugggg

SEQ ID NO: 521 - RNA, 23 nt, Artificial Sequence
  Targeting domain
auuugcauugagauagugugggg

SEQ ID NO: 522 - RNA, 24 nt, Artificial Sequence
  Targeting domain
uauuugcauugagauagugugggg

SEQ ID NO: 523 - RNA, 18 nt, Artificial Sequence
  Targeting domain
aggcuggccaacccaugg

SEQ ID NO: 524 - RNA, 19 nt, Artificial Sequence
  Targeting domain
aaggcuggccaacccaugg

SEQ ID NO: 525 - RNA, 20 nt, Artificial Sequence
  Targeting domain
caaggcuggccaacccaugg

SEQ ID NO: 526 - RNA, 21 nt, Artificial Sequence
  Targeting domain
gcaaggcuggccaacccaugg

SEQ ID NO: 527 - RNA, 22 nt, Artificial Sequence
  Targeting domain
ggcaaggcuggccaacccaugg

SEQ ID NO: 528 - RNA, 23 nt, Artificial Sequence
  Targeting domain
aggcaaggcuggccaacccaugg

SEQ ID NO: 529 - RNA, 24 nt, Artificial Sequence
  Targeting domain
aaggcaaggcuggccaacccaugg

SEQ ID NO: 530 - RNA, 18 nt, Artificial Sequence
  Targeting domain
agcgaguguguggaacug

SEQ ID NO: 531 - RNA, 19 nt, Artificial Sequence
  Targeting domain
aagcgaguguguggaacug

SEQ ID NO: 532 - RNA, 20 nt, Artificial Sequence
  Targeting domain
gaagcgaguguguggaacug

SEQ ID NO: 533 - RNA, 21 nt, Artificial Sequence
  Targeting domain
agaagcgaguguguggaacug

SEQ ID NO: 534 - RNA, 22 nt, Artificial Sequence
  Targeting domain
cagaagcgaguguguggaacug

SEQ ID NO: 535 - RNA, 23 nt, Artificial Sequence
  Targeting domain
ccagaagcgaguguguggaacug

SEQ ID NO: 536 - RNA, 24 nt, Artificial Sequence
  Targeting domain
uccagaagcgaguguguggaacug

SEQ ID NO: 537 - RNA, 18 nt, Artificial Sequence
  Targeting domain
auuugcauugagauagug

SEQ ID NO: 538 - RNA, 19 nt, Artificial Sequence
  Targeting domain
uauuugcauugagauagug

SEQ ID NO: 539 - RNA, 21 nt, Artificial Sequence
  Targeting domain
gauauuugcauugagauagug

SEQ ID NO: 540 - RNA, 22 nt, Artificial Sequence
  Targeting domain
agauauuugcauugagauagug

SEQ ID NO: 541 - RNA, 23 nt, Artificial Sequence
  Targeting domain
cagauauuugcauugagauagug

SEQ ID NO: 542 - RNA, 24 nt, Artificial Sequence
  Targeting domain
acagauauuugcauugagauagug

SEQ ID NO: 543 - RNA, 18 nt, Artificial Sequence
  Targeting domain
guuccagaagcgagugug

SEQ ID NO: 544 - RNA, 19 nt, Artificial Sequence
  Targeting domain
cguuccagaagcgagugug

SEQ ID NO: 545 - RNA, 21 nt, Artificial Sequence
  Targeting domain
gacguuccagaagcgagugug

SEQ ID NO: 546 - RNA, 22 nt, Artificial Sequence
  Targeting domain
agacguuccagaagcgagugug

SEQ ID NO: 547 - RNA, 23 nt, Artificial Sequence
  Targeting domain
cagacguuccagaagcgagugug

SEQ ID NO: 548 - RNA, 24 nt, Artificial Sequence
  Targeting domain
ucagacguuccagaagcgagugug

SEQ ID NO: 549 - RNA, 18 nt, Artificial Sequence
  Targeting domain
uugcauugagauagugug

SEQ ID NO: 550 - RNA, 19 nt, Artificial Sequence
  Targeting domain
uuugcauugagauagugug

SEQ ID NO: 551 - RNA, 21 nt, Artificial Sequence
  Targeting domain
uauuugcauugagauagugug

SEQ ID NO: 552 - RNA, 22 nt, Artificial Sequence
  Targeting domain
auauuugcauugagauagugug

SEQ ID NO: 553 - RNA, 23 nt, Artificial Sequence
  Targeting domain
gauauuugcauugagauagugug

SEQ ID NO: 554 - RNA, 24 nt, Artificial Sequence
  Targeting domain
agauauuugcauugagauagugug

SEQ ID NO: 555 - RNA, 18 nt, Artificial Sequence
  Targeting domain
uauuugcauugagauagu

SEQ ID NO: 556 - RNA, 19 nt, Artificial Sequence
  Targeting domain
auauuugcauugagauagu

SEQ ID NO: 557 - RNA, 20 nt, Artificial Sequence
  Targeting domain
gauauuugcauugagauagu

SEQ ID NO: 558 - RNA, 21 nt, Artificial Sequence
  Targeting domain
agauauuugcauugagauagu

SEQ ID NO: 559 - RNA, 22 nt, Artificial Sequence
  Targeting domain
cagauauuugcauugagauagu

SEQ ID NO: 560 - RNA, 23 nt, Artificial Sequence
  Targeting domain
acagauauuugcauugagauagu

SEQ ID NO: 561 - RNA, 24 nt, Artificial Sequence
  Targeting domain
gacagauauuugcauugagauagu

SEQ ID NO: 562 - RNA, 18 nt, Artificial Sequence
  Targeting domain
cguuccagaagcgagugu

SEQ ID NO: 563 - RNA, 19 nt, Artificial Sequence
  Targeting domain
acguuccagaagcgagugu

SEQ ID NO: 564 - RNA, 20 nt, Artificial Sequence
  Targeting domain
gacguuccagaagcgagugu

SEQ ID NO: 565 - RNA, 21 nt, Artificial Sequence
  Targeting domain
agacguuccagaagcgagugu

SEQ ID NO: 566 - RNA, 22 nt, Artificial Sequence
  Targeting domain
cagacguuccagaagcgagugu

SEQ ID NO: 567 - RNA, 23 nt, Artificial Sequence
  Targeting domain
ucagacguuccagaagcgagugu

SEQ ID NO: 568 - RNA, 24 nt, Artificial Sequence
  Targeting domain
cucagacguuccagaagcgagugu

SEQ ID NO: 569 - RNA, 18 nt, Artificial Sequence
  Targeting domain
uuugcauugagauagugu

SEQ ID NO: 570 - RNA, 19 nt, Artificial Sequence
  Targeting domain
auuugcauugagauagugu

SEQ ID NO: 571 - RNA, 21 nt, Artificial Sequence
  Targeting domain
auauuugcauugagauagugu

SEQ ID NO: 572 - RNA, 22 nt, Artificial Sequence
  Targeting domain
gauauuugcauugagauagugu

SEQ ID NO: 573 - RNA, 23 nt, Artificial Sequence
  Targeting domain
agauauuugcauugagauagugu

SEQ ID NO: 574 - RNA, 24 nt, Artificial Sequence
  Targeting domain
cagauauuugcauugagauagugu

SEQ ID NO: 575 - RNA, 18 nt, Artificial Sequence
  Targeting domain
gaauaaauuagagaaaaa

SEQ ID NO: 576 - RNA, 19 nt, Artificial Sequence
  Targeting domain
agaauaaauuagagaaaaa

SEQ ID NO: 577 - RNA, 20 nt, Artificial Sequence
  Targeting domain
aagaauaaauuagagaaaaa

SEQ ID NO: 578 - RNA, 21 nt, Artificial Sequence
  Targeting domain
gaagaauaaauuagagaaaaa

SEQ ID NO: 579 - RNA, 22 nt, Artificial Sequence
  Targeting domain
ggaagaauaaauuagagaaaaa

SEQ ID NO: 580 - RNA, 23 nt, Artificial Sequence
  Targeting domain
gggaagaauaaauuagagaaaaa

SEQ ID NO: 581 - RNA, 24 nt, Artificial Sequence
  Targeting domain
agggaagaauaaauuagagaaaaa

SEQ ID NO: 582 - RNA, 18 nt, Artificial Sequence
  Targeting domain
cuagggaugaagaauaaa

SEQ ID NO: 583 - RNA, 19 nt, Artificial Sequence
  Targeting domain
gcuagggaugaagaauaaa

SEQ ID NO: 584 - RNA, 21 nt, Artificial Sequence
  Targeting domain
uggcuagggaugaagaauaaa

SEQ ID NO: 585 - RNA, 22 nt, Artificial Sequence
  Targeting domain
cuggcuagggaugaagaauaaa

SEQ ID NO: 586 - RNA, 23 nt, Artificial Sequence
  Targeting domain
gcuggcuagggaugaagaauaaa

SEQ ID NO: 587 - RNA, 24 nt, Artificial Sequence
  Targeting domain
ggcuggcuagggaugaagaauaaa

SEQ ID NO: 588 - RNA, 18 nt, Artificial Sequence
  Targeting domain
agaaggaaacuagcuaaa

SEQ ID NO: 589 - RNA, 19 nt, Artificial Sequence
  Targeting domain
gagaaggaaacuagcuaaa

SEQ ID NO: 590 - RNA, 21 nt, Artificial Sequence
  Targeting domain
gggagaaggaaacuagcuaaa

SEQ ID NO: 591 - RNA, 22 nt, Artificial Sequence
  Targeting domain
ugggagaaggaaacuagcuaaa

SEQ ID NO: 592 - RNA, 23 nt, Artificial Sequence
  Targeting domain
augggagaaggaaacuagcuaaa

SEQ ID NO: 593 - RNA, 24 nt, Artificial Sequence
  Targeting domain
gaugggagaaggaaacuagcuaaa

SEQ ID NO: 594 - RNA, 18 nt, Artificial Sequence
  Targeting domain
aaaacuggaaugacugaa

SEQ ID NO: 595 - RNA, 19 nt, Artificial Sequence
  Targeting domain
aaaaacuggaaugacugaa

SEQ ID NO: 596 - RNA, 20 nt, Artificial Sequence
  Targeting domain
gaaaaacuggaaugacugaa

SEQ ID NO: 597 - RNA, 21 nt, Artificial Sequence
  Targeting domain
agaaaaacuggaaugacugaa

SEQ ID NO: 598 - RNA, 22 nt, Artificial Sequence
  Targeting domain
gagaaaaacuggaaugacugaa

SEQ ID NO: 599 - RNA, 23 nt, Artificial Sequence
  Targeting domain
agagaaaaacuggaaugacugaa

SEQ ID NO: 600 - RNA, 24 nt, Artificial Sequence
  Targeting domain
uagagaaaaacuggaaugacugaa

SEQ ID NO: 601 - RNA, 18 nt, Artificial Sequence
  Targeting domain
gcuagggaugaagaauaa

SEQ ID NO: 602 - RNA, 19 nt, Artificial Sequence
  Targeting domain
ggcuagggaugaagaauaa

SEQ ID NO: 603 - RNA, 20 nt, Artificial Sequence
  Targeting domain
uggcuagggaugaagaauaa

SEQ ID NO: 604 - RNA, 21 nt, Artificial Sequence
  Targeting domain
cuggcuagggaugaagaauaa

SEQ ID NO: 605 - RNA, 22 nt, Artificial Sequence
  Targeting domain
gcuggcuagggaugaagaauaa

SEQ ID NO: 606 - RNA, 23 nt, Artificial Sequence
  Targeting domain
ggcuggcuagggaugaagaauaa

SEQ ID NO: 607 - RNA, 24 nt, Artificial Sequence
  Targeting domain
cggcuggcuagggaugaagaauaa

SEQ ID NO: 608 - RNA, 18 nt, Artificial Sequence
  Targeting domain
gagaaggaaacuagcuaa

SEQ ID NO: 609 - RNA, 19 nt, Artificial Sequence
  Targeting domain
ggagaaggaaacuagcuaa

SEQ ID NO: 610 - RNA, 21 nt, Artificial Sequence
  Targeting domain
ugggagaaggaaacuagcuaa

SEQ ID NO: 611 - RNA, 22 nt, Artificial Sequence
  Targeting domain
augggagaaggaaacuagcuaa

SEQ ID NO: 612 - RNA, 23 nt, Artificial Sequence
  Targeting domain
gaugggagaaggaaacuagcuaa

SEQ ID NO: 613 - RNA, 24 nt, Artificial Sequence
  Targeting domain
ugaugggagaaggaaacuagcuaa

SEQ ID NO: 614 - RNA, 18 nt, Artificial Sequence
  Targeting domain
auagucuuagaguaucca

SEQ ID NO: 615 - RNA, 19 nt, Artificial Sequence
  Targeting domain
aauagucuuagaguaucca

SEQ ID NO: 616 - RNA, 20 nt, Artificial Sequence
  Targeting domain
caauagucuuagaguaucca

SEQ ID NO: 617 - RNA, 21 nt, Artificial Sequence
  Targeting domain
ccaauagucuuagaguaucca

SEQ ID NO: 618 - RNA, 22 nt, Artificial Sequence
  Targeting domain
accaauagucuuagaguaucca

SEQ ID NO: 619 - RNA, 23 nt, Artificial Sequence
  Targeting domain
gaccaauagucuuagaguaucca

SEQ ID NO: 620 - RNA, 24 nt, Artificial Sequence
  Targeting domain
ugaccaauagucuuagaguaucca

SEQ ID NO: 621 - RNA, 18 nt, Artificial Sequence
  Targeting domain
auccucuaugaugggaga

SEQ ID NO: 622 - RNA, 19 nt, Artificial Sequence
  Targeting domain
uauccucuaugaugggaga

SEQ ID NO: 623 - RNA, 21 nt, Artificial Sequence
  Targeting domain
gguauccucuaugaugggaga

SEQ ID NO: 624 - RNA, 22 nt, Artificial Sequence
  Targeting domain
ugguauccucuaugaugggaga

SEQ ID NO: 625 - RNA, 23 nt, Artificial Sequence
  Targeting domain
cugguauccucuaugaugggaga

SEQ ID NO: 626 - RNA, 24 nt, Artificial Sequence
  Targeting domain
ccugguauccucuaugaugggaga

SEQ ID NO: 627 - RNA, 18 nt, Artificial Sequence
  Targeting domain
uccugguauccucuauga

SEQ ID NO: 628 - RNA, 19 nt, Artificial Sequence
  Targeting domain
guccugguauccucuauga

SEQ ID NO: 629 - RNA, 21 nt, Artificial Sequence
  Targeting domain
aaguccugguauccucuauga

SEQ ID NO: 630 - RNA, 22 nt, Artificial Sequence
  Targeting domain
gaaguccugguauccucuauga

SEQ ID NO: 631 - RNA, 23 nt, Artificial Sequence
  Targeting domain
agaaguccugguauccucuauga

SEQ ID NO: 632 - RNA, 24 nt, Artificial Sequence
  Targeting domain
aagaaguccugguauccucuauga

SEQ ID NO: 633 - RNA, 18 nt, Artificial Sequence
  Targeting domain
ggagaaggaaacuagcua

SEQ ID NO: 634 - RNA, 19 nt, Artificial Sequence
  Targeting domain
gggagaaggaaacuagcua

SEQ ID NO: 635 - RNA, 20 nt, Artificial Sequence
  Targeting domain
ugggagaaggaaacuagcua

SEQ ID NO: 636 - RNA, 21 nt, Artificial Sequence
  Targeting domain
augggagaaggaaacuagcua

SEQ ID NO: 637 - RNA, 22 nt, Artificial Sequence
  Targeting domain
gaugggagaaggaaacuagcua

SEQ ID NO: 638 - RNA, 23 nt, Artificial Sequence
  Targeting domain
ugaugggagaaggaaacuagcua

SEQ ID NO: 639 - RNA, 24 nt, Artificial Sequence
  Targeting domain
augaugggagaaggaaacuagcua

SEQ ID NO: 640 - RNA, 18 nt, Artificial Sequence
  Targeting domain
aaagggaagaauaaauua

SEQ ID NO: 641 - RNA, 19 nt, Artificial Sequence
  Targeting domain
uaaagggaagaauaaauua

SEQ ID NO: 642 - RNA, 20 nt, Artificial Sequence
  Targeting domain
cuaaagggaagaauaaauua

SEQ ID NO: 643 - RNA, 21 nt, Artificial Sequence
  Targeting domain
gcuaaagggaagaauaaauua

SEQ ID NO: 644 - RNA, 22 nt, Artificial Sequence
  Targeting domain
agcuaaagggaagaauaaauua

SEQ ID NO: 645 - RNA, 23 nt, Artificial Sequence
  Targeting domain
uagcuaaagggaagaauaaauua

SEQ ID NO: 646 - RNA, 24 nt, Artificial Sequence
  Targeting domain
cuagcuaaagggaagaauaaauua

SEQ ID NO: 647 - RNA, 18 nt, Artificial Sequence
  Targeting domain
agaguauccagugaggcc

SEQ ID NO: 648 - RNA, 19 nt, Artificial Sequence
  Targeting domain
uagaguauccagugaggcc

SEQ ID NO: 649 - RNA, 21 nt, Artificial Sequence
  Targeting domain
cuuagaguauccagugaggcc

SEQ ID NO: 650 - RNA, 22 nt, Artificial Sequence
  Targeting domain
ucuuagaguauccagugaggcc

SEQ ID NO: 651 - RNA, 23 nt, Artificial Sequence
  Targeting domain
gucuuagaguauccagugaggcc

SEQ ID NO: 652 - RNA, 24 nt, Artificial Sequence
  Targeting domain
agucuuagaguauccagugaggcc

SEQ ID NO: 653 - RNA, 18 nt, Artificial Sequence
  Targeting domain
uagaguauccagugaggc

SEQ ID NO: 654 - RNA, 19 nt, Artificial Sequence
  Targeting domain
uuagaguauccagugaggc

SEQ ID NO: 655 - RNA, 20 nt, Artificial Sequence
  Targeting domain
cuuagaguauccagugaggc

SEQ ID NO: 656 - RNA, 21 nt, Artificial Sequence
  Targeting domain
ucuuagaguauccagugaggc

SEQ ID NO: 657 - RNA, 22 nt, Artificial Sequence
  Targeting domain
gucuuagaguauccagugaggc

SEQ ID NO: 658 - RNA, 23 nt, Artificial Sequence
  Targeting domain
agucuuagaguauccagugaggc

SEQ ID NO: 659 - RNA, 24 nt, Artificial Sequence
  Targeting domain
uagucuuagaguauccagugaggc

SEQ ID NO: 660 - RNA, 18 nt, Artificial Sequence
  Targeting domain
caggggccggcggcuggc

SEQ ID NO: 661 - RNA, 19 nt, Artificial Sequence
  Targeting domain
ccaggggccggcggcuggc

SEQ ID NO: 662 - RNA, 20 nt, Artificial Sequence
  Targeting domain
gccaggggccggcggcuggc

SEQ ID NO: 663 - RNA, 21 nt, Artificial Sequence
  Targeting domain
ggccaggggccggcggcuggc

SEQ ID NO: 664 - RNA, 22 nt, Artificial Sequence
  Targeting domain
aggccaggggccggcggcuggc

SEQ ID NO: 665 - RNA, 23 nt, Artificial Sequence
  Targeting domain
gaggccaggggccggcggcuggc

SEQ ID NO: 666 - RNA, 24 nt, Artificial Sequence
  Targeting domain
ugaggccaggggccggcggcuggc

SEQ ID NO: 667 - RNA, 18 nt, Artificial Sequence
  Targeting domain
aaaauuagcaguauccuc

SEQ ID NO: 668 - RNA, 19 nt, Artificial Sequence
  Targeting domain
aaaaauuagcaguauccuc

SEQ ID NO: 669 - RNA, 20 nt, Artificial Sequence
  Targeting domain
aaaaaauuagcaguauccuc

SEQ ID NO: 670 - RNA, 21 nt, Artificial Sequence
  Targeting domain
aaaaaaauuagcaguauccuc

SEQ ID NO: 671 - RNA, 22 nt, Artificial Sequence
  Targeting domain
aaaaaaaauuagcaguauccuc

SEQ ID NO: 672 - RNA, 23 nt, Artificial Sequence
  Targeting domain
uaaaaaaaauuagcaguauccuc

SEQ ID NO: 673 - RNA, 24 nt, Artificial Sequence
  Targeting domain
auaaaaaaaauuagcaguauccuc

SEQ ID NO: 674 - RNA, 18 nt, Artificial Sequence
  Targeting domain
guuccacacacucgcuuc

SEQ ID NO: 675 - RNA, 19 nt, Artificial Sequence
  Targeting domain
aguuccacacacucgcuuc

SEQ ID NO: 676 - RNA, 21 nt, Artificial Sequence
  Targeting domain
gcaguuccacacacucgcuuc

SEQ ID NO: 677 - RNA, 22 nt, Artificial Sequence
  Targeting domain
agcaguuccacacacucgcuuc

SEQ ID NO: 678 - RNA, 23 nt, Artificial Sequence
  Targeting domain
cagcaguuccacacacucgcuuc

SEQ ID NO: 679 - RNA, 24 nt, Artificial Sequence
  Targeting domain
ucagcaguuccacacacucgcuuc

SEQ ID NO: 680 - RNA, 18 nt, Artificial Sequence
  Targeting domain
uauccucuaugaugggag

SEQ ID NO: 681 - RNA, 19 nt, Artificial Sequence
  Targeting domain
guauccucuaugaugggag

SEQ ID NO: 682 - RNA, 20 nt, Artificial Sequence
  Targeting domain
gguauccucuaugaugggag

SEQ ID NO: 683 - RNA, 21 nt, Artificial Sequence
  Targeting domain
ugguauccucuaugaugggag

SEQ ID NO: 684 - RNA, 22 nt, Artificial Sequence
  Targeting domain
cugguauccucuaugaugggag

SEQ ID NO: 685 - RNA, 23 nt, Artificial Sequence
  Targeting domain
ccugguauccucuaugaugggag

SEQ ID NO: 686 - RNA, 24 nt, Artificial Sequence
  Targeting domain
uccugguauccucuaugaugggag

SEQ ID NO: 687 - RNA, 18 nt, Artificial Sequence
  Targeting domain
gccggcggcuggcuaggg

SEQ ID NO: 688 - RNA, 19 nt, Artificial Sequence
  Targeting domain
ggccggcggcuggcuaggg

SEQ ID NO: 689 - RNA, 20 nt, Artificial Sequence
  Targeting domain
gggccggcggcuggcuaggg

SEQ ID NO: 690 - RNA, 21 nt, Artificial Sequence
  Targeting domain
ggggccggcggcuggcuaggg

SEQ ID NO: 691 - RNA, 22 nt, Artificial Sequence
  Targeting domain
aggggccggcggcuggcuaggg

SEQ ID NO: 692 - RNA, 23 nt, Artificial Sequence
  Targeting domain
caggggccggcggcuggcuaggg

SEQ ID NO: 693 - RNA, 24 nt, Artificial Sequence
  Targeting domain
ccaggggccggcggcuggcuaggg

SEQ ID NO: 694 - RNA, 18 nt, Artificial Sequence
  Targeting domain
ugguauccucuaugaugg

SEQ ID NO: 695 - RNA, 19 nt, Artificial Sequence
  Targeting domain
cugguauccucuaugaugg

SEQ ID NO: 696 - RNA, 20 nt, Artificial Sequence
  Targeting domain
ccugguauccucuaugaugg

SEQ ID NO: 697 - RNA, 21 nt, Artificial Sequence
  Targeting domain
uccugguauccucuaugaugg

SEQ ID NO: 698 - RNA, 22 nt, Artificial Sequence
  Targeting domain
guccugguauccucuaugaugg

SEQ ID NO: 699 - RNA, 23 nt, Artificial Sequence
  Targeting domain
aguccugguauccucuaugaugg

SEQ ID NO: 700 - RNA, 24 nt, Artificial Sequence
  Targeting domain
aaguccugguauccucuaugaugg

SEQ ID NO: 701 - RNA, 18 nt, Artificial Sequence
  Targeting domain
guccugguauccucuaug

SEQ ID NO: 702 - RNA, 19 nt, Artificial Sequence
  Targeting domain
aguccugguauccucuaug

SEQ ID NO: 703 - RNA, 20 nt, Artificial Sequence
  Targeting domain
aaguccugguauccucuaug

SEQ ID NO: 704 - RNA, 21 nt, Artificial Sequence
  Targeting domain
gaaguccugguauccucuaug

SEQ ID NO: 705 - RNA, 22 nt, Artificial Sequence
  Targeting domain
agaaguccugguauccucuaug

SEQ ID NO: 706 - RNA, 23 nt, Artificial Sequence
  Targeting domain
aagaaguccugguauccucuaug

SEQ ID NO: 707 - RNA, 24 nt, Artificial Sequence
  Targeting domain
aaagaaguccugguauccucuaug

SEQ ID NO: 708 - RNA, 18 nt, Artificial Sequence
  Targeting domain
cuaaagggaagaauaaau

SEQ ID NO: 709 - RNA, 19 nt, Artificial Sequence
  Targeting domain
gcuaaagggaagaauaaau

SEQ ID NO: 710 - RNA, 20 nt, Artificial Sequence
  Targeting domain
agcuaaagggaagaauaaau

SEQ ID NO: 711 - RNA, 21 nt, Artificial Sequence
  Targeting domain
uagcuaaagggaagaauaaau

SEQ ID NO: 712 - RNA, 22 nt, Artificial Sequence
  Targeting domain
cuagcuaaagggaagaauaaau

SEQ ID NO: 713 - RNA, 23 nt, Artificial Sequence
  Targeting domain
acuagcuaaagggaagaauaaau

SEQ ID NO: 714 - RNA, 24 nt, Artificial Sequence
  Targeting domain
aacuagcuaaagggaagaauaaau

SEQ ID NO: 715 - RNA, 18 nt, Artificial Sequence
  Targeting domain
aaacuggaaugacugaau

SEQ ID NO: 716 - RNA, 19 nt, Artificial Sequence
  Targeting domain
aaaacuggaaugacugaau

SEQ ID NO: 717 - RNA, 21 nt, Artificial Sequence
  Targeting domain
gaaaaacuggaaugacugaau

SEQ ID NO: 718 - RNA, 22 nt, Artificial Sequence
  Targeting domain
agaaaaacuggaaugacugaau

SEQ ID NO: 719 - RNA, 23 nt, Artificial Sequence
  Targeting domain
gagaaaaacuggaaugacugaau

SEQ ID NO: 720 - RNA, 24 nt, Artificial Sequence
  Targeting domain
agagaaaaacuggaaugacugaau

SEQ ID NO: 721 - RNA, 18 nt, Artificial Sequence
  Targeting domain
ccugguauccucuaugau

SEQ ID NO: 722 - RNA, 19 nt, Artificial Sequence
  Targeting domain
uccugguauccucuaugau

SEQ ID NO: 723 - RNA, 21 nt, Artificial Sequence
  Targeting domain
aguccugguauccucuaugau

SEQ ID NO: 724 - RNA, 22 nt, Artificial Sequence
  Targeting domain
aaguccugguauccucuaugau

SEQ ID NO: 725 - RNA, 23 nt, Artificial Sequence
  Targeting domain
gaaguccugguauccucuaugau

SEQ ID NO: 726 - RNA, 24 nt, Artificial Sequence
  Targeting domain
agaaguccugguauccucuaugau

SEQ ID NO: 727 - RNA, 18 nt, Artificial Sequence
  Targeting domain
aaauuagcaguauccucu

SEQ ID NO: 728 - RNA, 19 nt, Artificial Sequence
  Targeting domain
aaaauuagcaguauccucu

SEQ ID NO: 729 - RNA, 21 nt, Artificial Sequence
  Targeting domain
aaaaaauuagcaguauccucu

SEQ ID NO: 730 - RNA, 22 nt, Artificial Sequence
  Targeting domain
aaaaaaauuagcaguauccucu

SEQ ID NO: 731 - RNA, 23 nt, Artificial Sequence
  Targeting domain
aaaaaaaauuagcaguauccucu

SEQ ID NO: 732 - RNA, 24 nt, Artificial Sequence
  Targeting domain
uaaaaaaaauuagcaguauccucu

SEQ ID NO: 733 - RNA, 18 nt, Artificial Sequence
  Targeting domain
cacucgcuucuggaacgu

SEQ ID NO: 734 - RNA, 19 nt, Artificial Sequence
  Targeting domain
acacucgcuucuggaacgu

SEQ ID NO: 735 - RNA, 20 nt, Artificial Sequence
  Targeting domain
cacacucgcuucuggaacgu

SEQ ID NO: 736 - RNA, 21 nt, Artificial Sequence
  Targeting domain
acacacucgcuucuggaacgu

SEQ ID NO: 737 - RNA, 22 nt, Artificial Sequence
  Targeting domain
cacacacucgcuucuggaacgu

SEQ ID NO: 738 - RNA, 23 nt, Artificial Sequence
  Targeting domain
ccacacacucgcuucuggaacgu

SEQ ID NO: 739 - RNA, 24 nt, Artificial Sequence
  Targeting domain
uccacacacucgcuucuggaacgu

SEQ ID NO: 740 - RNA, 18 nt, Artificial Sequence
  Targeting domain
cucaaugcaaauaucugu

SEQ ID NO: 741 - RNA, 19 nt, Artificial Sequence
  Targeting domain
ucucaaugcaaauaucugu

SEQ ID NO: 742 - RNA, 20 nt, Artificial Sequence
  Targeting domain
aucucaaugcaaauaucugu

SEQ ID NO: 743 - RNA, 21 nt, Artificial Sequence
  Targeting domain
uaucucaaugcaaauaucugu

SEQ ID NO: 744 - RNA, 22 nt, Artificial Sequence
  Targeting domain
cuaucucaaugcaaauaucugu

SEQ ID NO: 745 - RNA, 23 nt, Artificial Sequence
  Targeting domain
acuaucucaaugcaaauaucugu

SEQ ID NO: 746 - RNA, 24 nt, Artificial Sequence
  Targeting domain
cacuaucucaaugcaaauaucugu

SEQ ID NO: 747 - RNA, 18 nt, Artificial Sequence
  Targeting domain
aguuccacacacucgcuu

SEQ ID NO: 748 - RNA, 19 nt, Artificial Sequence
  Targeting domain
caguuccacacacucgcuu

SEQ ID NO: 749 - RNA, 20 nt, Artificial Sequence
  Targeting domain
gcaguuccacacacucgcuu

SEQ ID NO: 750 - RNA, 21 nt, Artificial Sequence
  Targeting domain
agcaguuccacacacucgcuu

SEQ ID NO: 751 - RNA, 22 nt, Artificial Sequence
  Targeting domain
cagcaguuccacacacucgcuu

SEQ ID NO: 752 - RNA, 23 nt, Artificial Sequence
  Targeting domain
ucagcaguuccacacacucgcuu

SEQ ID NO: 753 - RNA, 24 nt, Artificial Sequence
  Targeting domain
uucagcaguuccacacacucgcuu

SEQ ID NO: 754 - RNA, 18 nt, Artificial Sequence
  Targeting domain
aauuagcaguauccucuu

SEQ ID NO: 755 - RNA, 19 nt, Artificial Sequence
  Targeting domain
aaauuagcaguauccucuu

SEQ ID NO: 756 - RNA, 21 nt, Artificial Sequence
  Targeting domain
aaaaauuagcaguauccucuu

SEQ ID NO: 757 - RNA, 22 nt, Artificial Sequence
  Targeting domain
aaaaaauuagcaguauccucuu

SEQ ID NO: 758 - RNA, 23 nt, Artificial Sequence
  Targeting domain
aaaaaaauuagcaguauccucuu

SEQ ID NO: 759 - RNA, 24 nt, Artificial Sequence
  Targeting domain
aaaaaaaauuagcaguauccucuu

SEQ ID NO: 760 - RNA, 17 nt, Artificial Sequence
  Targeting domain
gaagaaaacuagcuaaa

SEQ ID NO: 761 - RNA, 17 nt, Artificial Sequence
  Targeting domain
gcagcaguauccucuug

SEQ ID NO: 762 - RNA, 20 nt, Artificial Sequence
  Targeting domain
ggagaagaaaacuagcuaaa

SEQ ID NO: 763 - RNA, 20 nt, Artificial Sequence
  Targeting domain
gggagaagaaaacuagcuaa

SEQ ID NO: 764 - RNA, 20 nt, Artificial Sequence
  Targeting domain
guccugguaucuucuauggu

SEQ ID NO: 765 - RNA, 17 nt, Artificial Sequence
  Targeting domain
agaagaaaacuagcuaa

SEQ ID NO: 766 - RNA, 17 nt, Artificial Sequence
  Targeting domain
aguccugguaucuucua

SEQ ID NO: 767 - RNA, 17 nt, Artificial Sequence
  Targeting domain
ccaccauagaagauacc

SEQ ID NO: 768 - RNA, 17 nt, Artificial Sequence
  Targeting domain
ccugguaucuucuaugg

SEQ ID NO: 769 - RNA, 17 nt, Artificial Sequence
  Targeting domain
cagcaguauccucuugg

SEQ ID NO: 770 - RNA, 17 nt, Artificial Sequence
  Targeting domain
aauuggaaugacugaau

SEQ ID NO: 771 - RNA, 17 nt, Artificial Sequence
  Targeting domain
cugguaucuucuauggu

SEQ ID NO: 772 - RNA, 17 nt, Artificial Sequence
  Targeting domain
agcagcaguauccucuu

SEQ ID NO: 773 - RNA, 20 nt, Artificial Sequence
  Targeting domain
agaaguccugguaucuucua

SEQ ID NO: 774 - RNA, 20 nt, Artificial Sequence
  Targeting domain
cucccaccauagaagauacc

SEQ ID NO: 775 - RNA, 20 nt, Artificial Sequence
  Targeting domain
aguccugguaucuucuaugg

SEQ ID NO: 776 - RNA, 20 nt, Artificial Sequence
  Targeting domain
aagcagcaguauccucuugg

SEQ ID NO: 777 - RNA, 20 nt, Artificial Sequence
  Targeting domain
uaagcagcaguauccucuug

SEQ ID NO: 778 - RNA, 20 nt, Artificial Sequence
  Targeting domain
agaauaaauuagagaaaaau

SEQ ID NO: 779 - RNA, 20 nt, Artificial Sequence
  Targeting domain
aaaaauuggaaugacugaau

SEQ ID NO: 780 - RNA, 20 nt, Artificial Sequence
  Targeting domain
auuaagcagcaguauccucu

SEQ ID NO: 781 - RNA, 20 nt, Artificial Sequence
  Targeting domain
uuaagcagcaguauccucuu

SEQ ID NO: 782 - RNA, 17 nt, Artificial Sequence
  Targeting domain
auaaauuagagaaaaau

SEQ ID NO: 783 - RNA, 17 nt, Artificial Sequence
  Targeting domain
aagcagcaguauccucu

SEQ ID NO: 784 - RNA, 20 nt, Artificial Sequence
  Targeting domain
gaagaaaacuagcuaaaggg

SEQ ID NO: 785 - RNA, 22 nt, Artificial Sequence
  Targeting domain
gagaagaaaacuagcuaaaggg

SEQ ID NO: 786 - RNA, 23 nt, Artificial Sequence
  Targeting domain
ggagaagaaaacuagcuaaaggg

SEQ ID NO: 787 - RNA, 24 nt, Artificial Sequence
  Targeting domain
gggagaagaaaacuagcuaaaggg

SEQ ID NO: 788 - RNA, 19 nt, Artificial Sequence
  Targeting domain
gaauaaauuagagaaaaau

SEQ ID NO: 789 - RNA, 22 nt, Artificial Sequence
  Targeting domain
gaagaauaaauuagagaaaaau

SEQ ID NO: 790 - RNA, 23 nt, Artificial Sequence
  Targeting domain
ggaagaauaaauuagagaaaaau

SEQ ID NO: 791 - RNA, 24 nt, Artificial Sequence
  Targeting domain
gggaagaauaaauuagagaaaaau

SEQ ID NO: 792 - RNA, 18 nt, Artificial Sequence
  Targeting domain
agagaaaaauuggaauga

SEQ ID NO: 793 - RNA, 19 nt, Artificial Sequence
  Targeting domain
uagagaaaaauuggaauga

SEQ ID NO: 794 - RNA, 20 nt, Artificial Sequence
  Targeting domain
uuagagaaaaauuggaauga

SEQ ID NO: 795 - RNA, 21 nt, Artificial Sequence
  Targeting domain
auuagagaaaaauuggaauga

SEQ ID NO: 796 - RNA, 22 nt, Artificial Sequence
  Targeting domain
aauuagagaaaaauuggaauga

SEQ ID NO: 797 - RNA, 23 nt, Artificial Sequence
  Targeting domain
aaauuagagaaaaauuggaauga

SEQ ID NO: 798 - RNA, 24 nt, Artificial Sequence
  Targeting domain
uaaauuagagaaaaauuggaauga

SEQ ID NO: 799 - RNA, 18 nt, Artificial Sequence
  Targeting domain
agaaaacuagcuaaaggg

SEQ ID NO: 800 - RNA, 19 nt, Artificial Sequence
  Targeting domain
aagaaaacuagcuaaaggg

SEQ ID NO: 801 - RNA, 21 nt, Artificial Sequence
  Targeting domain
agaagaaaacuagcuaaaggg

SEQ ID NO: 802 - RNA, 18 nt, Artificial Sequence
  Targeting domain
aauaaauuagagaaaaau

SEQ ID NO: 803 - RNA, 21 nt, Artificial Sequence
  Targeting domain
aagaauaaauuagagaaaaau

SEQ ID NO: 804 - RNA, 18 nt, Artificial Sequence
  Targeting domain
aguuuucuucucccacca

SEQ ID NO: 805 - RNA, 19 nt, Artificial Sequence
  Targeting domain
uaguuuucuucucccacca

SEQ ID NO: 806 - RNA, 20 nt, Artificial Sequence
  Targeting domain
cuaguuuucuucucccacca

SEQ ID NO: 807 - RNA, 21 nt, Artificial Sequence
  Targeting domain
gcuaguuuucuucucccacca

SEQ ID NO: 808 - RNA, 22 nt, Artificial Sequence
  Targeting domain
agcuaguuuucuucucccacca

SEQ ID NO: 809 - RNA, 23 nt, Artificial Sequence
  Targeting domain
uagcuaguuuucuucucccacca

SEQ ID NO: 810 - RNA, 24 nt, Artificial Sequence
  Targeting domain
uuagcuaguuuucuucucccacca

SEQ ID NO: 811 - RNA, 18 nt, Artificial Sequence
  Targeting domain
ucccaccauagaagauac

SEQ ID NO: 812 - RNA, 19 nt, Artificial Sequence
  Targeting domain
cucccaccauagaagauac

SEQ ID NO: 813 - RNA, 20 nt, Artificial Sequence
  Targeting domain
ucucccaccauagaagauac

SEQ ID NO: 814 - RNA, 21 nt, Artificial Sequence
  Targeting domain
uucucccaccauagaagauac

SEQ ID NO: 815 - RNA, 22 nt, Artificial Sequence
  Targeting domain
cuucucccaccauagaagauac

SEQ ID NO: 816 - RNA, 23 nt, Artificial Sequence
  Targeting domain
ucuucucccaccauagaagauac

SEQ ID NO: 817 - RNA, 24 nt, Artificial Sequence
  Targeting domain
uucuucucccaccauagaagauac

SEQ ID NO: 818 - RNA, 18 nt, Artificial Sequence
  Targeting domain
agaagaaaacuagcuaaa

SEQ ID NO: 819 - RNA, 19 nt, Artificial Sequence
  Targeting domain
gagaagaaaacuagcuaaa

SEQ ID NO: 820 - RNA, 21 nt, Artificial Sequence
  Targeting domain
gggagaagaaaacuagcuaaa

SEQ ID NO: 821 - RNA, 22 nt, Artificial Sequence
  Targeting domain
ugggagaagaaaacuagcuaaa

SEQ ID NO: 822 - RNA, 23 nt, Artificial Sequence
  Targeting domain
gugggagaagaaaacuagcuaaa

SEQ ID NO: 823 - RNA, 24 nt, Artificial Sequence
  Targeting domain
ggugggagaagaaaacuagcuaaa

SEQ ID NO: 824 - RNA, 18 nt, Artificial Sequence
  Targeting domain
aaaauuggaaugacugaa

SEQ ID NO: 825 - RNA, 19 nt, Artificial Sequence
  Targeting domain
aaaaauuggaaugacugaa

SEQ ID NO: 826 - RNA, 20 nt, Artificial Sequence
  Targeting domain
gaaaaauuggaaugacugaa

SEQ ID NO: 827 - RNA, 21 nt, Artificial Sequence
  Targeting domain
agaaaaauuggaaugacugaa

SEQ ID NO: 828 - RNA, 22 nt, Artificial Sequence
  Targeting domain
gagaaaaauuggaaugacugaa

SEQ ID NO: 829 - RNA, 23 nt, Artificial Sequence
  Targeting domain
agagaaaaauuggaaugacugaa

SEQ ID NO: 830 - RNA, 24 nt, Artificial Sequence
  Targeting domain
uagagaaaaauuggaaugacugaa

SEQ ID NO: 831 - RNA, 18 nt, Artificial Sequence
  Targeting domain
gagaagaaaacuagcuaa

SEQ ID NO: 832 - RNA, 19 nt, Artificial Sequence
  Targeting domain
ggagaagaaaacuagcuaa

SEQ ID NO: 833 - RNA, 21 nt, Artificial Sequence
  Targeting domain
ugggagaagaaaacuagcuaa

SEQ ID NO: 834 - RNA, 22 nt, Artificial Sequence
  Targeting domain
gugggagaagaaaacuagcuaa

SEQ ID NO: 835 - RNA, 23 nt, Artificial Sequence
  Targeting domain
ggugggagaagaaaacuagcuaa

SEQ ID NO: 836 - RNA, 24 nt, Artificial Sequence
  Targeting domain
uggugggagaagaaaacuagcuaa

SEQ ID NO: 837 - RNA, 18 nt, Artificial Sequence
  Targeting domain
ggagaagaaaacuagcua

SEQ ID NO: 838 - RNA, 19 nt, Artificial Sequence
  Targeting domain
gggagaagaaaacuagcua

SEQ ID NO: 839 - RNA, 20 nt, Artificial Sequence
  Targeting domain
ugggagaagaaaacuagcua

SEQ ID NO: 840 - RNA, 21 nt, Artificial Sequence
  Targeting domain
gugggagaagaaaacuagcua

SEQ ID NO: 841 - RNA, 22 nt, Artificial Sequence
  Targeting domain
ggugggagaagaaaacuagcua

SEQ ID NO: 842 - RNA, 23 nt, Artificial Sequence
  Targeting domain
uggugggagaagaaaacuagcua

SEQ ID NO: 843 - RNA, 24 nt, Artificial Sequence
  Targeting domain
auggugggagaagaaaacuagcua

SEQ ID NO: 844 - RNA, 18 nt, Artificial Sequence
  Targeting domain
uuaagcagcaguauccuc

SEQ ID NO: 845 - RNA, 19 nt, Artificial Sequence
  Targeting domain
auuaagcagcaguauccuc

SEQ ID NO: 846 - RNA, 20 nt, Artificial Sequence
  Targeting domain
aauuaagcagcaguauccuc

SEQ ID NO: 847 - RNA, 21 nt, Artificial Sequence
  Targeting domain
aaauuaagcagcaguauccuc

SEQ ID NO: 848 - RNA, 22 nt, Artificial Sequence
  Targeting domain
aaaauuaagcagcaguauccuc

SEQ ID NO: 849 - RNA, 23 nt, Artificial Sequence
  Targeting domain
aaaaauuaagcagcaguauccuc

SEQ ID NO: 850 - RNA, 24 nt, Artificial Sequence
  Targeting domain
aaaaaauuaagcagcaguauccuc

SEQ ID NO: 851 - RNA, 18 nt, Artificial Sequence
  Targeting domain
uaucuucuauggugggag

SEQ ID NO: 852 - RNA, 19 nt, Artificial Sequence
  Targeting domain
guaucuucuauggugggag

SEQ ID NO: 853 - RNA, 20 nt, Artificial Sequence
  Targeting domain
gguaucuucuauggugggag

SEQ ID NO: 854 - RNA, 21 nt, Artificial Sequence
  Targeting domain
ugguaucuucuauggugggag

SEQ ID NO: 855 - RNA, 22 nt, Artificial Sequence
  Targeting domain
cugguaucuucuauggugggag

SEQ ID NO: 856 - RNA, 23 nt, Artificial Sequence
  Targeting domain
ccugguaucuucuauggugggag

SEQ ID NO: 857 - RNA, 24 nt, Artificial Sequence
  Targeting domain
uccugguaucuucuauggugggag

SEQ ID NO: 858 - RNA, 18 nt, Artificial Sequence
  Targeting domain
uccugguaucuucuaugg

SEQ ID NO: 859 - RNA, 19 nt, Artificial Sequence
  Targeting domain
guccugguaucuucuaugg

SEQ ID NO: 860 - RNA, 21 nt, Artificial Sequence
  Targeting domain
aaguccugguaucuucuaugg

SEQ ID NO: 861 - RNA, 22 nt, Artificial Sequence
  Targeting domain
gaaguccugguaucuucuaugg

SEQ ID NO: 862 - RNA, 23 nt, Artificial Sequence
  Targeting domain
agaaguccugguaucuucuaugg

SEQ ID NO: 863 - RNA, 24 nt, Artificial Sequence
  Targeting domain
aagaaguccugguaucuucuaugg

SEQ ID NO: 864 - RNA, 18 nt, Artificial Sequence
  Targeting domain
ugguaucuucuauggugg

SEQ ID NO: 865 - RNA, 19 nt, Artificial Sequence
  Targeting domain
cugguaucuucuauggugg

SEQ ID NO: 866 - RNA, 20 nt, Artificial Sequence
  Targeting domain
ccugguaucuucuauggugg

SEQ ID NO: 867 - RNA, 21 nt, Artificial Sequence
  Targeting domain
uccugguaucuucuauggugg

SEQ ID NO: 868 - RNA, 22 nt, Artificial Sequence
  Targeting domain
guccugguaucuucuauggugg

SEQ ID NO: 869 - RNA, 23 nt, Artificial Sequence
  Targeting domain
aguccugguaucuucuauggugg

SEQ ID NO: 870 - RNA, 24 nt, Artificial Sequence
  Targeting domain
aaguccugguaucuucuauggugg

SEQ ID NO: 871 - RNA, 18 nt, Artificial Sequence
  Targeting domain
guccugguaucuucuaug

SEQ ID NO: 872 - RNA, 19 nt, Artificial Sequence
  Targeting domain
aguccugguaucuucuaug

SEQ ID NO: 873 - RNA, 20 nt, Artificial Sequence
  Targeting domain
aaguccugguaucuucuaug

SEQ ID NO: 874 - RNA, 21 nt, Artificial Sequence
  Targeting domain
gaaguccugguaucuucuaug

SEQ ID NO: 875 - RNA, 22 nt, Artificial Sequence
  Targeting domain
agaaguccugguaucuucuaug

SEQ ID NO: 876 - RNA, 23 nt, Artificial Sequence
  Targeting domain
aagaaguccugguaucuucuaug

SEQ ID NO: 877 - RNA, 24 nt, Artificial Sequence
  Targeting domain
aaagaaguccugguaucuucuaug

SEQ ID NO: 878 - RNA, 18 nt, Artificial Sequence
  Targeting domain
aaauuggaaugacugaau

SEQ ID NO: 879 - RNA, 19 nt, Artificial Sequence
  Targeting domain
aaaauuggaaugacugaau

SEQ ID NO: 880 - RNA, 21 nt, Artificial Sequence
  Targeting domain
gaaaaauuggaaugacugaau

SEQ ID NO: 881 - RNA, 22 nt, Artificial Sequence
  Targeting domain
agaaaaauuggaaugacugaau

SEQ ID NO: 882 - RNA, 23 nt, Artificial Sequence
  Targeting domain
gagaaaaauuggaaugacugaau

SEQ ID NO: 883 - RNA, 24 nt, Artificial Sequence
  Targeting domain
agagaaaaauuggaaugacugaau

SEQ ID NO: 884 - RNA, 18 nt, Artificial Sequence
  Targeting domain
uaagcagcaguauccucu

SEQ ID NO: 885 - RNA, 19 nt, Artificial Sequence
  Targeting domain
uuaagcagcaguauccucu

SEQ ID NO: 886 - RNA, 21 nt, Artificial Sequence
  Targeting domain
aauuaagcagcaguauccucu

SEQ ID NO: 887 - RNA, 22 nt, Artificial Sequence
  Targeting domain
aaauuaagcagcaguauccucu

SEQ ID NO: 888 - RNA, 23 nt, Artificial Sequence
  Targeting domain
aaaauuaagcagcaguauccucu

SEQ ID NO: 889 - RNA, 24 nt, Artificial Sequence
  Targeting domain
aaaaauuaagcagcaguauccucu

SEQ ID NO: 890 - RNA, 18 nt, Artificial Sequence
  Targeting domain
ccugguaucuucuauggu

SEQ ID NO: 891 - RNA, 19 nt, Artificial Sequence
  Targeting domain
uccugguaucuucuauggu

SEQ ID NO: 892 - RNA, 21 nt, Artificial Sequence
  Targeting domain
aguccugguaucuucuauggu

SEQ ID NO: 893 - RNA, 22 nt, Artificial Sequence
  Targeting domain
aaguccugguaucuucuauggu

SEQ ID NO: 894 - RNA, 23 nt, Artificial Sequence
  Targeting domain
gaaguccugguaucuucuauggu

SEQ ID NO: 895 - RNA, 24 nt, Artificial Sequence
  Targeting domain
agaaguccugguaucuucuauggu

SEQ ID NO: 896 - RNA, 18 nt, Artificial Sequence
  Targeting domain
aagcagcaguauccucuu

SEQ ID NO: 897 - RNA, 19 nt, Artificial Sequence
  Targeting domain
uaagcagcaguauccucuu

SEQ ID NO: 898 - RNA, 21 nt, Artificial Sequence
  Targeting domain
auuaagcagcaguauccucuu

SEQ ID NO: 899 - RNA, 22 nt, Artificial Sequence
  Targeting domain
aauuaagcagcaguauccucuu

SEQ ID NO: 900 - RNA, 23 nt, Artificial Sequence
  Targeting domain
aaauuaagcagcaguauccucuu

SEQ ID NO: 901 - RNA, 24 nt, Artificial Sequence
  Targeting domain
aaaauuaagcagcaguauccucuu

SEQ ID NO: 902 - DNA, 4758 bp, Homo sapiens
  misc_feature (2716)..(2719): HPFH deletion site (4 bp del -225 to -222)
  misc_feature (2748)..(2753): GATA1 binding motif
  misc_feature (2762)..(2767): GATA1 binding motif
  misc_feature (2791)..(2799): FKLF transcription factor binding motif
  misc_feature (2823)..(2830): CP1/Coup TFII binding motif
  misc_feature (2824)..(2836): HPFH deletion site (13 bp del -114 to -102)
  misc_feature (2991)..(2993): Start codon
tttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccg

SEQ ID NO: 903 - DNA, 4773 bp, Homo sapiens
  misc_feature (1233)..(1238): GATA1 binding site
  misc_feature (2672)..(2677): GATA1 binding site
  misc_feature (2686)..(2691): GATA1 binding site
  misc_feature (2715)..(2723): FKLF transcription factor binding motif
  misc_feature (2747)..(2754): CP1/Coup TFII binding motif
  misc_feature (2748)..(2760): HPFH deletion site (13 bp del -114 to -102)
  misc_feature (2915)..(2917): Start codon
ttatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaa

SEQ ID NO: 904 - DNA, 87 bp, Artificial Sequence
  ssODN1 5′ homology arm
gggtgcttccttttattcttcatccctagccagccgccggcccctggcctcactggatactctaagactattggtcaagtttgcctt

SEQ ID NO: 905 - DNA, 89 bp, Artificial Sequence
  ssODN1 3′ homology arm
gtcaaggcaaggctggccaacccatgggtggagtttagccagggaccgtttcagacagatatttgcattgagatagtgtggggaagggg

SEQ ID NO: 906 - DNA, 176 bp, Artificial Sequence
  ssODN1
gggtgcttccttttattcttcatccctagccagccgccggcccctggcctcactggatactctaagactattggtcaagtttgccttgtcaaggcaaggctggccaacccatgggtggagtttagccagggaccgtttcagacagatatttgcattgagatagtgtggggaagggg

SEQ ID NO: 907 - DNA, 87 bp, Artificial Sequence; identical to SEQ ID NO: 904
  PhTx ssODN1 5′ homology arm
  misc_feature (1)..(1): Modified to contain phosphorothioate

SEQ ID NO: 908 - DNA, 89 bp, Artificial Sequence; identical to SEQ ID NO: 905
  PhTx ssODN1 3′ homology arm
  misc_feature (89)..(89): Modified to contain phosphorothioate

SEQ ID NO: 909 - DNA, 176 bp, Artificial Sequence; identical to SEQ ID NO: 906
  PhTx ssODN1
  misc_feature (1)..(1): Modified to contain phosphorothioate
  misc_feature (176)..(176): Modified to contain phosphorothioate

SEQ ID NO: 910 - DNA, 17 bp, Artificial Sequence
  targeting domain
ggctattggtcaaggca

SEQ ID NO: 911 - DNA, 20 bp, Artificial Sequence
  targeting domain
caaggctattggtcaaggca

SEQ ID NO: 912 - DNA, 17 bp, Artificial Sequence
  targeting domain
tgccttgtcaaggctat

SEQ ID NO: 913 - DNA, 20 bp, Artificial Sequence
  targeting domain
gtttgccttgtcaaggctat

SEQ ID NO: 914 - DNA, 17 bp, Artificial Sequence
  targeting domain
gaccaatagccttgaca

SEQ ID NO: 915 - DNA, 20 bp, Artificial Sequence
  targeting domain
cttgaccaatagccttgaca

SEQ ID NO: 916 - DNA, 17 bp, Artificial Sequence
  targeting domain
gtcaaggctattggtca

SEQ ID NO: 917 - DNA, 20 bp, Artificial Sequence
  targeting domain
cttgtcaaggctattggtca

SEQ ID NO: 918 - DNA, 17 bp, Artificial Sequence
  targeting domain
tcaagtttgccttgtca

SEQ ID NO: 919 - DNA, 20 bp, Artificial Sequence
  targeting domain
tggtcaagtttgccttgtca

SEQ ID NO: 920 - RNA, 20 nt, Artificial Sequence
  targeting domain plus PAM (NGG)
ggcuauuggucaaggcaagg

SEQ ID NO: 921 - RNA, 23 nt, Artificial Sequence
  targeting domain plus PAM (NGG)
caaggcuauuggucaaggcaagg

SEQ ID NO: 922 - RNA, 20 nt, Artificial Sequence
  targeting domain plus PAM (NGG)
ugccuugucaaggcuauugg

SEQ ID NO: 923 - RNA, 23 nt, Artificial Sequence
  targeting domain plus PAM (NGG)
guuugccuugucaaggcuauugg

SEQ ID NO: 924 - RNA, 20 nt, Artificial Sequence
  targeting domain plus PAM (NGG)
gaccaauagccuugacaagg

SEQ ID NO: 925 - RNA, 23 nt, Artificial Sequence
  targeting domain plus PAM (NGG)
cuugaccaauagccuugacaagg

SEQ ID NO: 926 - RNA, 20 nt, Artificial Sequence
  targeting domain plus PAM (NGG)
gucaaggcuauuggucaagg

SEQ ID NO: 927 - RNA, 23 nt, Artificial Sequence
  targeting domain plus PAM (NGG)
cuugucaaggcuauuggucaagg

SEQ ID NO: 928 - RNA, 20 nt, Artificial Sequence
  targeting domain plus PAM (NGG)
ucaaguuugccuugucaagg

SEQ ID NO: 929 - RNA, 23 nt, Artificial Sequence
  targeting domain plus PAM (NGG)
uggucaaguuugccuugucaagg

SEQ ID NO: 930 - DNA, 20 bp, Artificial Sequence
  targeting domain plus PAM (NGG)
ggctattggtcaaggcaagg

SEQ ID NO: 931 - DNA, 23 bp, Artificial Sequence
  targeting domain plus PAM (NGG)
caaggctattggtcaaggcaagg

SEQ ID NO: 932 - DNA, 20 bp, Artificial Sequence
  targeting domain plus PAM (NGG)
tgccttgtcaaggctattgg

SEQ ID NO: 933 - DNA, 23 bp, Artificial Sequence
  targeting domain plus PAM (NGG)
gtttgccttgtcaaggctattgg

SEQ ID NO: 934 - DNA, 20 bp, Artificial Sequence
  targeting domain plus PAM (NGG)
gaccaatagccttgacaagg

SEQ ID NO: 935 - DNA, 23 bp, Artificial Sequence
  targeting domain plus PAM (NGG)
cttgaccaatagccttgacaagg

SEQ ID NO: 936 - DNA, 20 bp, Artificial Sequence
  targeting domain plus PAM (NGG)
gtcaaggctattggtcaagg

SEQ ID NO: 937 - DNA, 23 bp, Artificial Sequence
  targeting domain plus PAM (NGG)
cttgtcaaggctattggtcaagg

SEQ ID NO: 938 - DNA, 20 bp, Artificial Sequence
  targeting domain plus PAM (NGG)
tcaagtttgccttgtcaagg

SEQ ID NO: 939 - DNA, 23 bp, Artificial Sequence
  targeting domain plus PAM (NGG)
tggtcaagtttgccttgtcaagg

SEQ ID NO: 940 - RNA, 17 nt, Artificial Sequence
  targeting domain
aguauccagugaggcca

SEQ ID NO: 941 - RNA, 20 nt, Artificial Sequence
  targeting domain
ggcaaggcuggccaacccau

SEQ ID NO: 942 - RNA, 20 nt, Artificial Sequence
  targeting domain
uauuugcauugagauagugu

SEQ ID NO: 943 - DNA, 17 bp, Artificial Sequence
  targeting domain
agtatccagtgaggcca

SEQ ID NO: 944 - DNA, 20 bp, Artificial Sequence
  targeting domain
ggcaaggctggccaacccat

SEQ ID NO: 945 - DNA, 20 bp, Artificial Sequence
  targeting domain
tatttgcattgagatagtgt

SEQ ID NO: 946 - RNA, 20 nt, Artificial Sequence
  targeting domain plus PAM (NGG)
aguauccagugaggccaggg

SEQ ID NO: 947 - RNA, 23 nt, Artificial Sequence
  targeting domain plus PAM (NGG)
ggcaaggcuggccaacccauggg

SEQ ID NO: 948 - RNA, 23 nt, Artificial Sequence
  targeting domain plus PAM (NGG)
uauuugcauugagauaguguggg

SEQ ID NO: 949 - DNA, 20 bp, Artificial Sequence
  targeting domain plus PAM (NGG)
agtatccagtgaggccaggg

SEQ ID NO: 950 - DNA, 23 bp, Artificial Sequence
  targeting domain plus PAM (NGG)
ggcaaggctggccaacccatggg

SEQ ID NO: 951 - DNA, 23 bp, Artificial Sequence
  targeting domain plus PAM (NGG)
tatttgcattgagatagtgtggg

SEQ ID NO: 952 - RNA, 20 nt, Artificial Sequence
  targeting domain
cuaacaguugcuuuuaucac

SEQ ID NO: 953 - RNA, 20 nt, Artificial Sequence
  targeting domain
gggcgugggugggguagaag

SEQ ID NO: 954 - RNA, 20 nt, Artificial Sequence
  targeting domain
cucuuagacauaacacacca

SEQ ID NO: 955 - RNA, 20 nt, Artificial Sequence
  targeting domain
aucagaggccaaacccuucc

SEQ ID NO: 956 - DNA, 20 bp, Artificial Sequence
  targeting domain
ctaacagttgcttttatcac

SEQ ID NO: 957 - DNA, 20 bp, Artificial Sequence
  targeting domain
gggcgtgggtggggtagaag

SEQ ID NO: 958 - DNA, 20 bp, Artificial Sequence
  targeting domain
ctcttagacataacacacca

SEQ ID NO: 959 - DNA, 20 bp, Artificial Sequence
  targeting domain
atcagaggccaaacccttcc

SEQ ID NO: 960 - RNA, 23 nt, Artificial Sequence
  targeting domain plus PAM (NGG)
cuaacaguugcuuuuaucacagg

SEQ ID NO: 961 - RNA, 23 nt, Artificial Sequence
  targeting domain plus PAM (NGG)
gggcgugggugggguagaagagg

SEQ ID NO: 962 - RNA, 23 nt, Artificial Sequence
  targeting domain plus PAM (NGG)
cucuuagacauaacacaccaggg

SEQ ID NO: 963 - RNA, 23 nt, Artificial Sequence
  targeting domain plus PAM (NGG)
aucagaggccaaacccuuccugg

SEQ ID NO: 964 - DNA, 23 bp, Artificial Sequence
  targeting domain plus PAM (NGG)
ctaacagttgcttttatcacagg

SEQ ID NO: 965 - DNA, 23 bp, Artificial Sequence
  targeting domain plus PAM (NGG)
gggcgtgggtggggtagaagagg

SEQ ID NO: 966 - DNA, 23 bp, Artificial Sequence
  targeting domain plus PAM (NGG)
ctcttagacataacacaccaggg

SEQ ID NO: 967 - DNA, 23 bp, Artificial Sequence
  targeting domain plus PAM (NGG)
atcagaggccaaacccttcctgg

SEQ ID NO: 968 - DNA, 115 bp, Artificial Sequence
  +58 DHS enhancer
caactttgaagctagtctagtgcaagctaacagttgcttttatcacaggctccaggaagggtttggcctctgattagggtgggggcgtgggtggggtagaagaggactggcagac

SEQ ID NO: 969 - DNA, 801 bp, Artificial Sequence
  +58 DHS enhancer region including ˜500 bp upstream and ˜200 bp downstream
aggtaccttttgtgtgtatgtgctgattgagggcccattgagaatattttgacttttagggaagctccaaactctcaaaccacagggatcacaacacatacgtgtgtctgttatgacgttatatgtaagcatcacaacaggcagagaatgtctgcaccccaccctggaaaacagcctgactgtgccccatgggcaaaccagactagtttatagggggttctactctgaggtactgatggaccttgggtgctattcctgtgataaggaaggcagctagacaggacttgggagttatctgtagtgagatggctgaaaagcgatacagggctggctctatgccccaggtgtgcataagtaagagcagatagctgattccagtgcaaagtccatacaggtaataacataggccagaaaagagatatggcatctactcttagacataacacaccagggtcaatacaactttgaagctagtctagtgcaagctaacagttgcttttatcacaggctccaggaagggtttggcctctgattagggtgggggcgtgggtggggtagaagaggactggcagacctctccatcggtggccgtttgcccaggggggcctctttcggaaggctctcttggtgatggagaattggattttatttctcaatgggaatgaaataatttgtatgccatgccgtgtggactcccaaaattgtaaaggaggtgaagcttcccctgtctgcactctcccctcctcataattgtccatttttcatctgtcgggctgtccacccatccatcacatataggca

SEQ ID NO: 970 - RNA, 100 nt, Artificial Sequence
  Unmodified Sp35 gRNA (OLI7066)
cuugucaaggcuauuggucaguuuuagagcuagaaauagcaaguuaaaauaaggcuaguccguuaucaacuugaaaaaguggcaccgagucggugcuuuu

SEQ ID NO: 971 - RNA, 100 nt, Artificial Sequence; identical to SEQ ID NO: 970
  Sp35 gRNA w/ 5′ PS (OLI8568)
  misc_feature (1)..(1): Modified to contain phosphorothioate
  misc_feature (2)..(2): Modified to contain phosphorothioate
  misc_feature (3)..(3): Modified to contain phosphorothioate

SEQ ID NO: 972 - RNA, 100 nt, Artificial Sequence; identical to SEQ ID NO: 970
  Sp35 gRNA w/ 5′ OMe (OLI8569)
  misc_feature (1)..(1): Modified to contain 2-o-methyl
  misc_feature (2)..(2): Modified to contain 2-o-methyl
  misc_feature (3)..(3): Modified to contain 2-o-methyl

SEQ ID NO: 973 - RNA, 100 nt, Artificial Sequence; identical to SEQ ID NO: 970
  Sp35 gRNA w/ 3′ PS/OMe (OLI8396)
  misc_feature (97)..(97): Modified to contain phosphorothioate, 2-o-methyl
  misc_feature (98)..(98): Modified to contain phosphorothioate, 2-o-methyl
  misc_feature (99)..(99): Modified to contain phosphorothioate, 2-o-methyl

SEQ ID NO: 974 - RNA, 100 nt, Artificial Sequence; identical to SEQ ID NO: 970
  Sp35 gRNA w/ 3′ PS/OMe (OLI8395)
  misc_feature (1)..(1): Modified to contain phosphorothioate, 2-o-methyl
  misc_feature (2)..(2): Modified to contain phosphorothioate, 2-o-methyl
  misc_feature (3)..(3): Modified to contain phosphorothioate, 2-o-methyl

SEQ ID NO: 975 - RNA, 100 nt, Artificial Sequence; identical to SEQ ID NO: 970
  Sp35 gRNA w/ 5′ and 3′ PS/OMe (OLI8394)
  misc_feature (1)..(1): Modified to contain phosphorothioate, 2-o-methyl
  misc_feature (2)..(2): Modified to contain phosphorothioate, 2-o-methyl
  misc_feature (3)..(3): Modified to contain phosphorothioate, 2-o-methyl
  misc_feature (97)..(97): Modified to contain phosphorothioate, 2-o-methyl
  misc_feature (98)..(98): Modified to contain phosphorothioate, 2-o-methyl
  misc_feature (99)..(99): Modified to contain phosphorothioate, 2-o-methyl

SEQ ID NO: 976 - RNA, 97 nt, Artificial Sequence
  Sp35 gRNA 97mer (OLI8018)
gucaaggcuauuggucaguuuuagagcuagaaauagcaaguuaaaauaaggcuaguccguuaucaacuugaaaaaguggcaccgagucggugcuuuu

SEQ ID NO: 977 - RNA, 98 nt, Artificial Sequence
  Sp35 gRNA 98mer (OLI8019)
ugucaaggcuauuggucaguuuuagagcuagaaauagcaaguuaaaauaaggcuaguccguuaucaacuugaaaaaguggcaccgagucggugcuuuu

SEQ ID NO: 978 - RNA, 99 nt, Artificial Sequence
  Sp35 gRNA 99mer (OLI8020)
uugucaaggcuauuggucaguuuuagagcuagaaauagcaaguuaaaauaaggcuaguccguuaucaacuugaaaaaguggcaccgagucggugcuuuu

SEQ ID NO: 979 - RNA, 100 nt, Artificial Sequence
  Sp35 gRNA w/ 5′ C->A (OLI8397)
auugucaaggcuauuggucaguuuuagagcuagaaauagcaaguuaaaauaaggcuaguccguuaucaacuugaaaaaguggcaccgagucggugcuuuu

SEQ ID NO: 980 - DNA, 100 bp, Artificial Sequence
  Unmodified Sp35 gRNA (OLI7066)
cttgtcaaggctattggtcagttttagagctagaaatagcaagttaaaataaggctagtccgttatcaacttgaaaaagtggcaccgagtcggtgctttt

SEQ ID NO: 981 - DNA, 100 bp, Artificial Sequence; identical to SEQ ID NO: 980
  Sp35 gRNA w/ 5′ PS (OLI8568)
  misc_feature (1)..(1): Modified to contain phosphorothioate
  misc_feature (2)..(2): Modified to contain phosphorothioate
  misc_feature (3)..(3): Modified to contain phosphorothioate

SEQ ID NO: 982 - DNA, 100 bp, Artificial Sequence; identical to SEQ ID NO: 980
  Sp35 gRNA w/ 5′ OMe (OLI8569)
  misc_feature (1)..(1): Modified to contain 2-o-methyl
  misc_feature (2)..(2): Modified to contain 2-o-methyl
  misc_feature (3)..(3): Modified to contain 2-o-methyl

SEQ ID NO: 983 - DNA, 100 bp, Artificial Sequence; identical to SEQ ID NO: 980
  Sp35 gRNA w/ 3′ PS/OMe (OLI8396)
  misc_feature (97)..(97): Modified to contain phosphorothioate, 2-o-methyl
  misc_feature (98)..(98): Modified to contain phosphorothioate, 2-o-methyl
  misc_feature (99)..(99): Modified to contain phosphorothioate, 2-o-methyl

SEQ ID NO: 984 - DNA, 100 bp, Artificial Sequence; identical to SEQ ID NO: 980
  Sp35 gRNA w/ 3′ PS/OMe (OLI8395)
  misc_feature (97)..(97): Modified to contain phosphorothioate, 2-o-methyl
  misc_feature (98)..(98): Modified to contain phosphorothioate, 2-o-methyl
  misc_feature (99)..(99): Modified to contain phosphorothioate, 2-o-methyl

SEQ ID NO: 985 - DNA, 100 bp, Artificial Sequence; identical to SEQ ID NO: 980
  Sp35 gRNA w/ 5′ and 3′ PS/OMe (OLI8394)
  misc_feature (1)..(1): Modified to contain phosphorothioate, 2-o-methyl
  misc_feature (2)..(2): Modified to contain phosphorothioate, 2-o-methyl
  misc_feature (3)..(3): Modified to contain phosphorothioate, 2-o-methyl
  misc_feature (97)..(97): Modified to contain phosphorothioate, 2-o-methyl
  misc_feature (98)..(98): Modified to contain phosphorothioate, 2-o-methyl
  misc_feature (99)..(99): Modified to contain phosphorothioate, 2-o-methyl

SEQ ID NO: 986 - DNA, 97 bp, Artificial Sequence
  Sp35 gRNA 97mer (OLI8018)
gtcaaggctattggtcagttttagagctagaaatagcaagttaaaataaggctagtccgttatcaacttgaaaaagtggcaccgagtcggtgctttt

SEQ ID NO: 987 - DNA, 98 bp, Artificial Sequence
  Sp35 gRNA 98mer (OLI8019)
tgtcaaggctattggtcagttttagagctagaaatagcaagttaaaataaggctagtccgttatcaacttgaaaaagtggcaccgagtcggtgctttt

SEQ ID NO: 988 - DNA, 99 bp, Artificial Sequence
  Sp35 gRNA 99mer (OLI8020)
ttgtcaaggctattggtcagttttagagctagaaatagcaagttaaaataaggctagtccgttatcaacttgaaaaagtggcaccgagtcggtgctttt

SEQ ID NO: 989 - DNA, 100 bp, Artificial Sequence
  Sp35 gRNA w/ 5′ C->A (OLI8397))
attgtcaaggctattggtcagttttagagctagaaatagcaagttaaaataaggctagtccgttatcaacttgaaaaagtggcaccgagtcggtgctttt

SEQ ID NO: 990 - DNA, 180 bp, Artificial Sequence
  PhTx ssODN 13nt - positive strand (OLI16414)
  misc_feature (1)..(1): Modified to contain phosphorothioate
  misc_feature (179)..(179): Modified to contain phosphorothioate
aagggtgcttccttttattcttcatccctagccagccgccggcccctggcctcactggatactctaagactattggtcaagtttgccttgtcaaggcaaggctggccaacccatgggtggagtttagccagggaccgtttcagacagatatttgcattgagatagtgtggggaaggggcc

SEQ ID NO: 991 - DNA, 90 bp, Artificial Sequence
  5′ homology arm: PhTx ssODN 13nt - positive strand (OLI16414)
  misc_feature (1)..(1): Modified to contain phosphorothioate
aagggtgcttccttttattcttcatccctagccagccgccggcccctggcctcactggatactctaagactattggtcaagtttgccttg

SEQ ID NO: 992 - DNA, 90 bp, Artificial Sequence
  3′ homology arm: PhTx ssODN 13nt - positive strand (OLI16414)
  misc_feature (89)..(89): Modified to contain phosphorothioate
tcaaggcaaggctggccaacccatgggtggagtttagccagggaccgtttcagacagatatttgcattgagatagtgtggggaaggggcc

SEQ ID NO: 993 - DNA, 180 bp, Artificial Sequence
  PhTx ssODN 13nt - negative strand (OLI16412)
  misc_feature (1)..(1): Modified to contain phosphorothioate
  misc_feature (179)..(179): Modified to contain phosphorothioate
ggccccttccccacactatctcaatgcaaatatctgtctgaaacggtccctggctaaactccacccatgggttggccagccttgccttgacaaggcaaacttgaccaatagtcttagagtatccagtgaggccaggggccggcggctggctagggatgaagaataaaaggaagcaccctt

SEQ ID NO: 994 - DNA, 90 bp, Artificial Sequence
  5′ homology arm: PhTx ssODN 13nt - negative strand (OLI16412)
  misc_feature (1)..(1): Modified to contain phosphorothioate
ggccccttccccacactatctcaatgcaaatatctgtctgaaacggtccctggctaaactccacccatgggttggccagccttgccttga

SEQ ID NO: 995 - DNA, 90 bp, Artificial Sequence
  3′ homology arm: PhTx ssODN 13nt - negative strand (OLI16412)
  misc_feature (89)..(89): Modified to contain phosphorothioate
caaggcaaacttgaccaatagtcttagagtatccagtgaggccaggggccggcggctggctagggatgaagaataaaaggaagcaccctt